(19) United States
(12) Patent Application Publication
(10) Pub. No.: US 2005/0256176 A1
(43) Pub. Date: Nov. 17, 2005

(54) Sulphonamide derivatives and their use as tace inhibitors

(76) Inventors: Jeremy Nicholas Burrows, Macclesfield (GB); Howard Tucker, Macclesfield (GB)

Correspondence Address: FISH & RICHARDSON P.C., P.O BOX 1022, MINNEAPOLIS, MN, 55440-1022, US

(21) Appl. No.: 10/527,349

(22) PCT Filed: Sep. 9, 2003
(86) PCT No.: PCT/GB03/03907

§ 371 Date: Mar. 10, 2005

(30) Foreign Application Priority Data
Sep. 13, 2002 (GB) 0221246.2

(51) Int. Cl. A61K 31/427; A61K 31/422; A61K 31/4164; C07D 417/02; C07D 413/02; C07D 43/02

(52) U.S. Cl. 514/369; 514/376; 514/389; 548/183; 548/227; 548/311.1; 548/319.1

(57) ABSTRACT

Sulphonamide derivatives that are useful in the inhibition of metalloproteinases and in particular in the inhibition of TNF-α Converting Enzyme (TACE).

The present invention relates to compounds useful in the inhibition of metalloproteinases and in particular to pharmaceutical compositions comprising these, as well as their use.

The compounds of this invention are inhibitors of one or more metalloproteinase enzymes and are particularly effective as inhibitors of TNF-α (Tumour Necrosis Factor-α) production. Metalloproteinases are a superfamily of proteinases (enzymes) whose numbers in recent years have increased dramatically. Based on structural and functional considerations these enzymes have been classified into families and subfamilies as described in N. M. Hooper (1994) FEBS Letters 354:1-6. Examples of metalloproteinases include the matrix metalloproteinases (MMP) such as the collagenases (MMP1, MMP8, MMP13), the gelatinases (2, MMP9), the stromelysins (MMP3, MMP10, MMP11), matrilysin (MMP7), metalloelastase (MMP12), enamelysin (MMP19), the MT-MMPs (MMP14, MMP15, MMP16, MMP17); the reprolysin or adamalysin or MDC family which includes the secretases and sheddases such as TNF-α converting enzymes (ADAM10 and TACE); the ADAM-TS family (for example ADAM-TS1 and ADAM-TS4); the astacin family which include enzymes such as procollagen processing proteinase (PCP); and other metalloproteinases such as the endothelin converting enzyme family and the angiotensin converting enzyme family.

Metalloproteinases are believed to be important in a plethora of physiological disease processes that involve tissue remodelling such as embryonic development, bone formation and uterine remodelling during menstruation. This is based on the ability of the metalloproteinases to cleave a broad range of matrix substrates such as collagen, proteoglycan and fibronectin. Metalloproteinases are also believed to be important in the processing, or secretion, of biologically important cell mediators, such as tumour necrosis factor-α (TNF-α); and the post translational proteolysis processing, or shedding, of biologically important membrane proteins, such as the low affinity IgE receptor CD23 (for a more complete list see N. M. Hooper et al., (1997) Biochem J. 321:265-279).

Metalloproteinases have been associated with many disease conditions. Inhibition of the activity of one or more metalloproteinases may well be of benefit in these disease conditions, for example: various inflammatory and allergic diseases such as, inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema and dermatitis); in tumour metastasis or invasion; in disease associated with uncontrolled degradation of the extracellular matrix such as osteoarthritis; in bone resorptive disease (such as osteoporosis and Paget's disease); in diseases associated with aberrant angiogenesis; the enhanced collagen remodelling associated with diabetes, periodontal disease (such as gingivitis), corneal ulceration, ulceration of the skin, post-operative conditions (such as colonic anastomosis) and dermal wound healing; demyelinating diseases of the central and peripheral nervous systems (such as multiple sclerosis); Alzheimer's disease; and extracellular matrix remodelling observed in cardiovascular diseases such as restenosis and atheroscelerosis.

A number of metalloproteinase inhibitors are known; different classes of compounds may have different degrees of potency and selectivity for inhibiting various metalloproteinases. We have discovered a class of compounds that are inhibitors of metalloproteinases and are of particular interest in inhibiting TACE. The compounds of this invention have beneficial potency and/or pharmacokinetic properties.

TACE (also known as ADAM17) which has been isolated and cloned [R. A. Black et al. (1997) Nature 385:729-733; M. L. Moss et al. (1997) Nature 385:733-736] is a member of the admalysin family of metalloproteins. TACE has been shown to be responsible for the cleavage of pro-TNF-α, a 26 kDa membrane bound protein to release 17 kDa biologically active soluble TNF-α. [Schlondorff et al. (2000) Biochem. J. 347: 131-138]. TACE mRNA is found in most tissues, however TNF-α is produced primarily by activated monocytes, macrophages and T lymphocytes. TNF-α has been implicated in a wide range of pro-inflammatory biological processes including induction of adhesion molecules and chemokines to promote cell trafficking, induction of matrix destroying enzymes, activation of fibroblasts to produce prostaglandins and activation of the immune system [Aggarwal et al (1996) Eur. Cytokine Netw. 7: 93-124]. Clinical use of the anti-TNF-α biologicals has shown TNF-α to play an important role in a range of inflammatory diseases including rheumatoid arthritis, Crohn's disease and psoriasis [Onrust et al (1998) Biodrugs 10: 397-422, Jarvis et al (1999) Drugs 57:945-964]. TACE activity has also been implicated in the shedding of other membrane bound proteins including TGFα, p75 & p55 TNF receptors, L-selectin and amyloid precursor protein [Black (2002) Int. J. Biochem. Cell Biol. 34: 1-5]. The biology of TACE inhibition has recently been reviewed and shows TACE to have a central role in TNF-α production and selective TACE inhibitors to have equal, and possibly greater, efficacy in the collagen induced arthritis model of RA than strategies that directly neutralise TNF-α [Newton et al (2001) Ann. Rheum. Dis. 60: iii25-iii32].

A TACE inhibitor might therefore be expected to show efficacy in all disease where TNF-α has been implicated including, but not limited to, inflammatory diseases including rheumatoid arthritis and psoriasis, autoimmune diseases, allergic/atopic diseases, transplant rejection and graft versus host disease, cardiovascular disease, reperfusion injury, malignancy and other proliferative diseases. A TACE inhibitor might also show efficacy in a respiratory disorder such as asthma or COPD.

Metalloproteinase inhibitors are known in the art. WO 02/096426 discloses hydantoin derivatives that are useful as inhibitors of metalloproteinases, TACE, aggrecanase or combinations thereof. The compounds disclosed therein comprises a hydantoin group and a phenyl group connected by a linking portion which differ from the present invention with regard to the linking portion. WO 02/074751 discloses compounds useful in the inhibition of metalloproteinases and in particular 1-(4-methyl-3,5-dioxoimidazolidin-4-yl)-N-[4-(4-chlorophenoxy)phenyl]methanesulphonamide which has been specifically disclaimed herein. The compounds of WO 02/074751 are particularly active against MMP12. WO 02/074750 also discloses metalloproteinase inhibitors.

We are able to provide compounds that have metalloproteinase inhibitory activity, and are in particular inhibitors of TACE (ADAM17).

According to the first aspect of the present invention there is provided a compound of formula (I), a pharmaceutically acceptable salt or in vivo hydrolysable ester thereof: 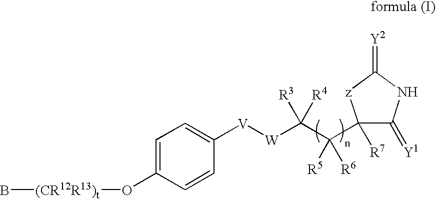
wherein:
Y¹ and Y² are independently O or S;
z is NR⁸, O or S;
n is 0 or 1;
W is NR¹, CR¹R² or a bond;
V is C(═O), NR¹⁵C(═O), NR¹⁵SO₂, SO₂ or a group of formula (A): 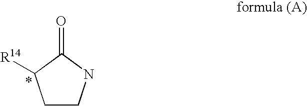
   where the group of formula (A) is bonded through nitrogen to W of formula (I) and through carbon * to phenyl of formula (I);
t is 0 or 1;
B is a group selected from aryl, heteroaryl and heterocyclyl where each group is optionally substituted by one or more groups independently selected from nitro, trifluoromethyl, trifluoromethoxy, halo, cyano, C₁₋₄alkyl (optionally substituted by R⁹ or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl and heterocyclyl whereby this group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl and C₁₋₄alkoxy; with the provisos that:
when V is a group of formula (A), C(═O), NR¹⁵C(═O) or NR¹⁵SO₂; or when V is SO₂ and n is 1 and W is NR¹, CR¹R² or a bond; or when V is SO₂ and n is 0 and W is CR¹R²; then B is a group selected from aryl, heteroaryl and heterocyclyl where each group is optionally substituted by one or more groups independently selected from nitro, trifluoromethyl, trifluoromethoxy, halo, cyano, C₁₋₄alkyl (optionally substituted by R⁹ or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl, heterocyclyl whereby this group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl and C₁₋₄alkoxy; and
when V is SO₂ and n is 0 and W is NR¹ or a bond; then B is a group selected from bicyclic aryl, bicyclic heteroaryl and bicyclic heterocyclyl, where each group is optionally substituted by one or more groups independently selected from nitro, trifluoromethyl, trifluoromethoxy, halo, cyano, C₁₋₄alkyl (optionally substituted by R⁹ or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl, heterocyclyl whereby this group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl and C₁₋₄alkoxy;
R¹ and R² are independently hydrogen or a group selected from C₁₋₆alkyl, C₂₋₆alkenyl, C₂₋₆alkynyl, C₃₋₆cycloalkyl and C₅₋₆cycloalkenyl where the group may be optionally substituted by halo, cyano, nitro, hydroxy or C₁₋₄alkoxy;
R³, R⁴, R⁵ and R⁶ are independently hydrogen or a group selected from C₁₋₆alkyl, C₂₋₆alkenyl, C₂₋₆alkynyl, C₃₋₆cycloalkyl, C₅₋₆cycloalkenyl, aryl, heteroaryl and heterocyclyl where the group is optionally substituted by one or more substituents independently selected from halo, nitro, cyano, trifluoromethyl, trifluoromethyloxy, C₁₋₄alkyl, C₂₋₄alkenyl, C₂₋₄alkynyl, C₃₋₆cycloalkyl (optionally substituted by one or more R¹⁷), aryl (optionally substituted by one or more R¹⁷), heteroaryl (optionally substituted by one or more R¹⁷), heterocyclyl, —OR¹⁸, —SR¹⁹, —SOR¹⁹, —SOR¹⁹, —COR¹⁹, —CO₂R¹⁸, CONR¹⁸R²⁰, —NR¹⁶COR¹⁸, —SO₂NR¹⁸R²⁰ and —NR¹⁶SO₂R¹⁹;
or R¹ and R³ together with the nitrogen or carbon and carbon to which they are respectively attached form a saturated 3-7-membered ring optionally containing 1 or 2 heteroatoms groups selected from NH, O, S, SO and SO₂ where the ring is optionally substituted on carbon or nitrogen by one or more C₁₋₄alkyl;
or R³ and R⁴ together form a saturated 3- to 7-membered ring optionally containing a heteroatom group selected from NH, O, S, SO and SO₂ where the ring is optionally substituted on carbon or nitrogen by one or more C₁₋₄alkyl;
or R³ and R⁵ together with the carbon atoms to which they are attached form a saturated 3- to 7-membered ring optionally containing a heteroatom group selected from NH, O, S, SO and SO₂ where the ring is optionally substituted on carbon or nitrogen by one or more C₁₋₄alkyl;
or R⁵ and R⁶ together form a saturated 3- to 7-membered ring optionally containing a heteroatom group selected from NH, O, S, SO and SO₂ where the ring is optionally substituted on carbon or nitrogen by one or more C₁₋₄alkyl;
R⁷ is hydrogen or a group selected from C₁₋₆alkyl, C₂₋₆alkenyl, C₂₋₆alkynyl, heteroalkyl, C₃₋₇cycloalkyl, aryl, heteroaryl or heterocyclyl where the group is optionally substituted by halo, C₁₋₄alkyl, C₁₋₄alkoxy, C₃₋₇cycloalkyl, heterocyclyl, aryl, heteroaryl and heteroalkyl; and wherein the group from which R⁷ may be selected is optionally substituted on the group and/or on its optional substituent by one or more substituents independently selected from halo, cyano, C₁₋₄alkyl, nitro, haloC₁₋₄alkyl, heteroalkyl, aryl, heteroaryl, hydroxyC₁₋₄alkyl, C₃₋₇cycloalkyl, heterocyclyl, C₁₋₄alkoxyC₁₋₄alkyl, haloC₁₋₄alkoxyC₁₋₄alkyl, carboxyC₁₋₄alkyl, —OR²¹, —CO₂R²¹, —SR²⁵, —SOR²⁵, —SO₂R²⁵, —NR²¹COR²², —CONR²¹R²² and —NHCONR²¹R²² or R³ and R⁷ together with the carbon atoms to which they are each attached and (CR⁵R⁶)ₙ form a saturated 5- to 7-membered ring optionally containing a heteroatom group selected from NH, O, S, SO and SO₂ where the ring is optionally substituted on carbon or nitrogen by one or more C₁₋₄alkyl;
R⁸ is selected from hydrogen, C₁₋₆alkyl and haloC₁₋₆alkyl;
R⁹ and R¹⁰ are independently hydrogen, C₁₋₆alkyl or C₃₋₆cycloalkyl;
or R⁹ and R¹⁰ together with the nitrogen to which they are attached form a heterocyclic 4 to 7-membered ring;
R¹¹ is C₁₋₆alkyl or C₃₋₆cycloalkyl;
R¹² and R¹³ are independently selected from hydrogen, C₁₋₆alkyl and C₃₋₆cycloalkyl;
R¹⁴ is hydrogen, —NR²³R²⁴ or C₁₋₄alkyl (optionally substituted by halo, —OR²³ and —NR²³R²⁴);
R⁶, R²³ and R²⁴ are independently hydrogen or C₁₋₆alkyl;
R¹⁷ is selected from halo, C₁₋₆alkyl, C₃₋₆cycloalkyl and C₁₋₆alkoxy;
R¹⁸ is hydrogen or a group selected from C₁₋₆alkyl, C₃₋₆cycloalkyl, C₅₋₆cycloalkenyl, saturated heterocyclyl, aryl, heteroaryl, arylC₁₋₄alkyl and heteroarylC₁₋₄alkyl where the group is optionally substituted by one or more halo;
R¹⁹ and R²⁵ are independently a group selected from C₁₋₆alkyl, C₃₋₆cycloalkyl, C₅₋₆cycloalkenyl, saturated heterocyclyl, aryl, heteroaryl, arylC₁₋₄alkyl and heteroarylC₁₋₄alkyl where the group is optionally substituted by one or more halo;
R²⁰ is hydrogen, C₁₋₆alkyl or C₃₋₆cycloalkyl;
or R¹⁸ and R²⁰ together with the nitrogen to which they are attached form a heterocyclic 4- to 7-membered ring;
R²¹ and R²² are independently hydrogen, C₁₋₄alkyl, haloC₁₋₄alkyl, aryl, arylC₁₋₄alkyl and benzoyl.

According to a second aspect of the invention there is provided a compound of formula (I), a pharmaceutically acceptable salt or in vivo hydrolysable ester thereof wherein:
Y¹ and Y² are independently O or S;
z is NR⁸, O or S;
n is 0;
W is NR¹ or a bond;
V is SO₂;
t is 0 or 1;
B is a group selected from aryl, heteroaryl and heterocyclyl where each group is optionally substituted by one or more groups independently selected from nitro, trifluoromethyl, trifluoromethoxy, halo, cyano, C₁₋₄alkyl (optionally substituted by R⁹ or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR⁹, —SOR¹¹, —SO₂R⁹, —SO₂NR⁹R¹⁰, —NR⁹SO₂R¹¹, —NHCONR⁹R¹⁰, —OR⁹, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl, heterocyclyl whereby this group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl and C₁₋₄alkoxy;
provided that when t is 0 and B is monocyclic aryl, monocyclic heteroaryl or monocyclic heterocyclyl then the monocyclic group that is B is substituted on the carbon or nitrogen adjacent to the atom to which the oxygen is attached, by a group described above;
R¹ is hydrogen or a group selected from C₁₋₄alkyl, C₂₋₄alkenyl, C₂₋₄alkynyl, C₃₋₅cycloalkyl and cyclopentenyl where the group may be optionally substituted by halo, cyano, nitro, hydroxy or C₁₋₄alkoxy;
R³ and R⁴ are independently hydrogen or a group selected from C₁₋₄alkyl, C₂₋₄alkenyl, C₂₋₄alkynyl, C₃₋₄cycloalkyl, cyclopentenyl, aryl, heteroaryl and heterocyclyl where the group is optionally substituted by one or more substituents independently selected from halo, nitro, cyano, trifluoromethyl, trifluoromethyloxy, C₁₋₄alkyl, C₂₋₄alkenyl, C₂₋₄alkynyl, C₃₋₆cycloalkyl (optionally substituted by one or more R¹⁷), aryl (optionally substituted by one or more R¹⁷), heteroaryl (optionally substituted by one or more R¹⁷), heterocyclyl, —OR¹⁸, —SR¹⁹, —SOR¹⁹, —SO₂R¹⁹, —CONR¹⁸R²⁰ and —NR¹⁶COR¹⁸;
or R¹ and R³ together with the nitrogen or carbon and carbon to which they are respectively attached form a saturated 3-7-membered ring optionally containing 1 or 2 heteroatoms groups selected form NH, O, S, SO and SO₂ where the ring is optionally substituted on carbon or nitrogen by one or more C₁₋₄alkyl;
or R³ and R⁴ together form a saturated 3- to 7-membered ring optionally containing a heteroatom group selected from NH, O, S, SO and SO₂ where the ring is optionally substituted on carbon or nitrogen by one or more C₁₋₄alkyl
R⁷ is hydrogen or a group selected from C₁₋₄alkyl, heteroalkyl, C₃₋₅cycloalkyl, aryl, heteroaryl or heterocyclyl where the group is optionally substituted by halo, C₁₋₄alkyl, C₁₋₄alkoxy, C₃₋₅cycloalkyl, heterocyclyl, aryl, heteroaryl and heteroalkyl; and wherein the group from which R⁷ may be selected is optionally substituted on the group and/or on its optional substituent by one or more substituents independently selected from halo, cyano, C₁₋₄alkyl, nitro, haloC₁₋₄alkyl, heteroalkyl, aryl, heteroaryl, hydroxyC₁₋₄alkyl, C₃₋₅cycloalkyl, heterocyclyl, C₁₋₄alkoxyC₁₋₄alkyl, haloC₁₋₄alkoxyC₁₋₄alkyl, carboxyC₁₋₄alkyl, —OR²¹, —CO₂R²¹, —SR²⁵, —SOR²⁵, —SO₂R²⁵, —CONR²¹R²² and —NHCONR²¹R²²;
or R³ and R⁷ together with the carbon atoms to which they are attached form a saturated 5- to 7-membered ring optionally containing a heteroatom group selected from NH, O, S and SO₂ where the ring is optionally substituted on carbon or nitrogen by one or more C₁₋₄alkyl;
R⁸ is selected from hydrogen, C₁₋₄alkyl and haloC₁₋₄alkyl;
R⁹ and R¹⁰ are independently hydrogen, C₁₋₄alkyl or C₃₋₅cycloalkyl;
or R⁹ and R¹⁰ together with the nitrogen to which they are attached form a heterocyclic 4 to 7-membered ring.
R¹¹ is C₁₋₄alkyl or C₃₋₅cycloalkyl;
R¹² and R¹³ are independently selected from hydrogen, C₁₋₄alkyl and C₃₋₄cycloalkyl;
R¹⁶ is hydrogen or C₁₋₄alkyl;
R¹⁷ is selected from halo, C₁₋₄alkyl, C₃₋₅cycloalkyl and C₁₋₄alkoxy;
R¹⁸ is hydrogen or a group selected from C₁₋₄alkyl, C₃₋₅cycloalkyl, C₅₋₆cycloalkenyl, saturated heterocyclyl, aryl, heteroaryl, arylC₁₋₄alkyl and heteroarylC₁₋₄alkyl where the group is optionally substituted by one or more halo;
R¹⁹ and R²⁵ are independently a group selected from C₁₋₄alkyl, C₃₋₅cycloalkyl, C₅₋₆cycloalkenyl, saturated heterocyclyl, aryl, heteroaryl, arylC₁₋₄alkyl and heteroarylC₁₋₄alkyl where the group is optionally substituted by one or more halo;
R²⁰ is hydrogen, C₁₋₄alkyl or C₃₋₅cycloalkyl;
or R¹⁸ and R²⁰ together with the nitrogen to which they are attached form a heterocyclic 4- to 6-membered ring;
R²¹ and R²² are independently hydrogen, C₁₋₄alkyl, haloC₁₋₄alkyl, aryl, arylC₁₋₄alkyl and benzoyl.

In particular the present invention provides a compound of formula (IA) or a pharmaceutically acceptable salt thereof: 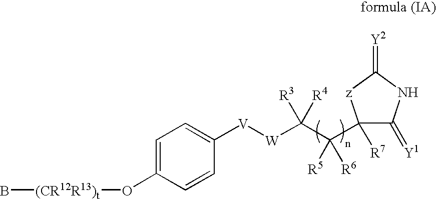
wherein:
Y¹ and Y² are both O;
z is NR⁸, O or S;
n is 0 or 1;
W is NR¹, CR¹R² or a bond;
V is NR¹⁵SO₂;
t is 0 or 1;
B is a group selected from aryl, heteroaryl and heterocyclyl where each group is optionally substituted by one or more groups independently selected from nitro, trifluoromethyl, trifluoromethoxy, halo, cyano, C₁₋₄alkyl (optionally substituted by R⁹ or C₁₋₄alkoxy or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl and heterocyclyl which group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl or C₁₋₄alkoxy;
R¹ and R² are independently hydrogen or a group selected from C₁₋₆alkyl, C₂₋₆alkenyl, C₂₋₆alkynyl, C₃₋₆cycloalkyl and C₅₋₆cycloalkenyl which group may be optionally substituted by halo, cyano, nitro, hydroxy or C₁₋₄alkoxy;
R³, R⁴, R⁵ and R⁶ are independently hydrogen or a group selected from C₁₋₆alkyl, C₂₋₆alkenyl, C₂₋₆alkynyl, C₃₋₆cycloalkyl, C₅₋₆cycloalkenyl, aryl, heteroaryl and heterocyclyl which group is optionally substituted by one or more substituents independently selected from halo, nitro, cyano, trifluoromethyl, trifluoromethoxy, C₁₋₄alkyl, C₂₋₄alkenyl, C₂₋₄alkynyl, C₃₋₆cycloalkyl (optionally substituted by one or more R¹⁷), aryl (optionally substituted by one or more R¹⁷), heteroaryl (optionally substituted by one or more R¹⁷), heterocyclyl, —OR¹⁸, —SR¹⁹, —SOR¹⁹, —SO₂R¹⁹, —COR¹⁹, —CO₂R¹⁸, —CONR¹⁸R²⁰, —NR¹⁶COR¹⁸, —SO₂NR¹⁸R²⁰ and —NR¹⁶SO₂R¹⁹;
or R¹ and R³ together with the nitrogen or carbon atoms and carbon atom to which they are respectively attached form a saturated 3- to 7-membered ring optionally containing 1 or 2 heteroatoms groups selected from NH, O, S, SO and SO₂ where the ring is optionally substituted on carbon by C₁₋₄alkyl, C₁₋₃alkoxy or fluoro and/or on nitrogen by —COC₁₋₃alkyl, —SO₂C₁₋₃alkyl or C₁₋₄alkyl;
or R³ and R⁴ together with the carbon atom to which they are attached form a saturated 3- to 7-membered ring optionally containing a heteroatom group selected from NH, O, S, SO and SO₂ where the ring is optionally substituted on carbon by C₁₋₄alkyl, C₁₋₃alkoxy or fluoro and/or on nitrogen by —COC₁₋₃alkyl, —SO₂C₁₋₃alkyl and/or C₁₋₄alkyl;
or R³ and R⁵ together with the carbon atoms to which they are attached form a saturated 3- to 7-membered ring optionally containing a heteroatom group selected from NH, O, S, SO and SO₂ where the ring is optionally substituted on carbon by C₁₋₄alkyl, C₁₋₃alkoxy or fluoro and/or on nitrogen by —COC₁₋₃alkyl, —SO₂C₁₋₃alkyl or C₁₋₄alkyl;
or R⁵ and R⁶ together with the carbon atom to which they are attached form a saturated 3- to 7-membered ring optionally containing a heteroatom group selected from NH, O, S, SO and SO₂ where the ring is optionally substituted on carbon by C₁₋₄alkyl, C₁₋₃alkoxy or fluoro and/or on nitrogen by —COC₁₋₃alkyl, —SO₂C₁₋₃alkyl or C₁₋₄alkyl;
R⁷ is hydrogen or a group selected from C₁₋₆alkyl, C₂₋₆alkenyl, C₂₋₄alkynyl, heteroalkyl, C₃₋₇cycloalkyl, aryl, heteroaryl and heterocyclyl where the group is optionally substituted by halo, C₁₋₄alkyl, C₁₋₄alkoxy, C₃₋₇cycloalkyl, heterocyclyl, aryl, heteroaryl or heteroalkyl; and wherein the group from which R⁷ may be selected is optionally substituted on the group and/or on its optional substituent by one or more substituents independently selected from halo, cyano, C₁₋₄alkyl, nitro, haloC₁₋₄alkyl, heteroalkyl, aryl, heteroaryl, hydroxyC₁₋₄alkyl, C₃₋₇cycloalkyl, heterocyclyl, C₁₋₄alkoxyC₁₋₄alkyl, haloC₁₋₄alkoxyC₁₋₄alkyl, —COC₁₋₄alkyl, —OR²¹, —NR²¹R²², —CO₂R²¹, —SR²⁵, —SOR²⁵, —SO₂R²⁵, —NR²¹COR²², —NR²¹CO₂R²², —CONR²¹R²² and —NHCONR²¹R²²;
or R³ and R⁷ together with the carbon atoms to which they are each attached and (CR⁵R⁶)ₙ form a saturated 5- to 7-membered ring optionally containing a heteroatom group selected from NH, O, S, SO and SO₂ where the ring is optionally substituted on carbon by C₁₋₄alkyl, C₁₋₃alkoxy or fluoro and/or on nitrogen by —COC₁₋₃alkyl, —SO₂C₁₋₃alkyl or C₁₋₄alkyl;
R⁸ is selected from hydrogen or methyl;
R⁹ and R¹⁰ are independently hydrogen, C₁₋₆alkyl or C₃₋₆cycloalkyl;
or R⁹ and R¹⁰ together with the nitrogen to which they are attached form a heterocyclic 4- to 7-membered ring;
R¹¹ is C₁₋₆alkyl or C₃₋₆cycloalkyl;
R¹² and R¹³ are independently selected from hydrogen, C₁₋₆alkyl and C₃₋₆cycloalkyl;
R¹⁵ is hydrogen or C₁₋₃alkyl;
R¹⁶ is hydrogen or C₁₋₆alkyl;
R¹⁷ is selected from halo, C₁₋₆alkyl, C₃₋₆cycloalkyl and C₁₋₆alkoxy;
R¹⁸ is hydrogen or a group selected from C₁₋₆alkyl, C₃₋₆cycloalkyl, C₅₋₆cycloalkenyl, saturated heterocyclyl, aryl, heteroaryl, arylC₁₋₄alkyl and heteroarylC₁₋₄alkyl where the group is optionally substituted by one or more halo;
R¹⁹ and R²⁵ are independently a group selected from C₁₋₆alkyl, C₃₋₆cycloalkyl, C₅₋₆cycloalkenyl, saturated heterocyclyl, aryl, heteroaryl, arylC₁₋₄alkyl and heteroarylC₁₋₄alkyl where the group is optionally substituted by one or more halo;
R²⁰ is hydrogen, C₁₋₆alkyl or C₃₋₆cycloalkyl;
or R¹⁸ and R²⁰ together with the nitrogen atom to which they are attached form a heterocyclic 4- to 7-membered ring;
R²¹ and R²² are independently hydrogen, C₁₋₄alkyl, haloC₁₋₄alkyl, aryl and arylC₁₋₄alkyl; provided a compound of formula (IA) is not 1-(4-methyl-2,5-dioxoimidazolidin-4-yl)-N-[4-(4-chlorophenoxy)phenyl]methanesulphonamide.

In addition, the invention also provides a compound of formula (IB) or a pharmaceutically acceptable salt thereof: 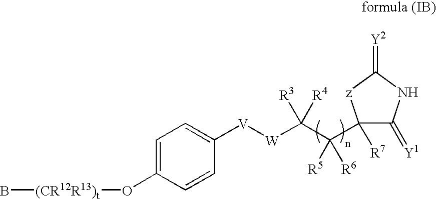
wherein:
Y¹ and Y² are independently O;
z is NR⁸, O or S;
n is 0 or 1;
W is NR¹;
V is SO₂ or CO;
t is 0 or 1;
B is a group selected from aryl, heteroaryl and heterocyclyl where each group is optionally substituted by one or more groups independently selected from nitro, trifluoromethyl, trifluoromethoxy, halo, cyano, C₁₋₄alkyl (optionally substituted by R⁹ or C₁₋₄alkoxy or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl and heterocyclyl which group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl or C₁₋₄alkoxy; provided that when t is 0 such that B is directly attached to the oxygen atom shown in formula (IB) and B is monocyclic aryl, monocyclic heteroaryl or monocyclic heterocyclyl and n is 0 then the monocyclic group that is B is substituted on one of the atoms that is adjacent to the atom to which the oxygen is attached, by a group selected from those listed above in the definition of B which optionally substitute B;
R¹ and R³ together with the nitrogen and carbon atoms to which they are respectively attached form a saturated 3- to 7-membered ring optionally containing a further heteroatom group selected from NH, O, S, SO and SO₂ where the ring is optionally substituted on carbon by C₁₋₄alkyl, fluoro or C₁₋₄alkoxy and/or on nitrogen by —COC₁₋₃alkyl, —SO₂C₁₋₃alkyl or C₁₋₄alkyl;
R⁴, R⁵ and R⁶ are independently hydrogen or a group selected from C₁₋₆alkyl, C₂₋₆alkenyl, C₂₋₆alkynyl, C₃₋₆cycloalkyl, C₅₋₆cycloalkenyl, aryl, heteroaryl and heterocyclyl which group is optionally substituted by one or more substituents independently selected from halo, nitro, cyano, trifluoromethyl, trifluoromethoxy, C₁₋₄alkyl, C₂₋₄alkenyl, C₂₋₄alkynyl, C₃₋₆cycloalkyl (optionally substituted by one or more R¹⁷), aryl (optionally substituted by one or more R¹⁷), heteroaryl (optionally substituted by one or more R¹⁷), heterocyclyl, —OR¹⁸, —SR¹⁹, —SOR¹⁹, —SO₂R¹⁹, —COR¹⁹, —CO₂R¹⁸, —CONR¹⁸R²⁰, —NR¹⁶COR¹⁸, —SO₂NR¹⁸R²⁰ and —NR¹⁶SO₂R¹⁹;
or R⁵ and R⁶ together with the carbon atom to which they are attached form a saturated 3- to 7-membered ring optionally containing a heteroatom group selected from NH, O, S, SO and SO₂ where the ring is optionally substituted on carbon by C₁₋₄alkyl, fluoro or C₁₋₄alkoxy and/or on nitrogen by —COC₁₋₃alkyl, —SO₂C₁₋₃alkyl or C₁₋₄alkyl;
R⁷ is hydrogen or a group selected from C₁₋₆alkyl, C₂₋₆alkenyl, C₂₋₆alkynyl, heteroalkyl, C₃₋₇cycloalkyl, aryl, heteroaryl or heterocyclyl where the group is optionally substituted by halo, C₁₋₄alkyl, C₁₋₄alkoxy, C₃₋₇cycloalkyl, heterocyclyl, aryl, heteroaryl and heteroalkyl; and wherein the group from which R⁷ may be selected is optionally substituted on the group and/or on its optional substituent by one or more substituents independently selected from halo, cyano, C₁₋₄alkyl, nitro, haloC₁₋₄alkyl, heteroalkyl, aryl, heteroaryl, hydroxyC₁₋₄alkyl, C₃₋₇cycloalkyl, heterocyclyl, C₁₋₄alkoxyC₁₋₄alkyl, haloC₁₋₄alkoxyC₁₋₄alkyl, —COC₁₋₄alkyl, —OR²¹, —NR²¹R²², —CO₂R²¹, —SR²⁵, —SOR²⁵, —SO₂R²⁵, —NR²¹COR²², —NR²¹CO₂R²², —CONR²¹R²² and —NHCONR²¹R²²;
R⁸ is selected from hydrogen or methyl;
R⁹ and R¹⁰ are independently hydrogen, C₁₋₆alkyl or C₃₋₆cycloalkyl;
or R⁹ and R¹⁰ together with the nitrogen to which they are attached form a heterocyclic 4- to 7-membered ring;
R¹¹ is C₁₋₆alkyl or C₃₋₆cycloalkyl;
R¹² and R¹³ are independently selected from hydrogen, C₁₋₆alkyl and C₃₋₆cycloalkyl;
R¹⁶ is hydrogen or C₁₋₆alkyl;
R¹⁷ is selected from halo, C₁₋₆alkyl, C₃₋₆cycloalkyl and C₁₋₆alkoxy;
R¹⁸ is hydrogen or a group selected from C₁₋₆alkyl, C₃₋₆cycloalkyl, C₅₋₆cycloalkenyl, saturated heterocyclyl, aryl, heteroaryl, arylC₁₋₄alkyl and heteroarylC₁₋₄alkyl where the group is optionally substituted by one or more halo;
R¹⁹ and R²⁵ are independently a group selected from C₁₋₆alkyl, C₃₋₆cycloalkyl, C₅₋₆cycloalkenyl, saturated heterocyclyl, aryl, heteroaryl, arylCl₁₋₄alkyl and heteroarylC₁₋₄alkyl where the group is optionally substituted by one or more halo;
R²⁰ is hydrogen, C₁₋₆alkyl or C₃₋₆cycloalkyl;
or R¹⁸ and R²⁰ together with the nitrogen to which they are attached form a heterocyclic 4- to 7-membered ring;
R²¹ and R²² are independently hydrogen, C₁₋₄alkyl, haloC₁₋₄alkyl, aryl and arylC₁₋₄alkyl.

It is to be understood that, insofar as certain of the compounds of the invention defined above may exist in optically active or racemic forms by virtue of one or more asymmetric carbon or sulphur atoms, the invention includes in its definition any such optically active or racemic form which possesses metalloproteinases inhibition activity and in particular TACE inhibition activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

Compounds of the invention are therefore provided as enantiomers, diastereomers, geometric isomers and atropisomers.

Within the present invention it is to be understood that a compound of the invention or a salt thereof may exhibit the phenomenon of tautomerism and that the formulae drawings within this specification can represent only one of the possible tautomeric forms. It is to be understood that the invention encompasses any tautomeric form which has metalloproteinases inhibition activity and in particular TACE inhibition activity and is not to be limited merely to any one tautomeric form utilised within the formulae drawings.

It is also to be understood that certain compounds of the invention and salts thereof can exist in solvated as well as unsolvated forms such as, for example, hydrated forms. It is to be understood that the invention encompasses all such solvated forms which have metalloproteinases inhibition activity and in particular TACE inhibition activity.

It is also to be understood that certain compounds of the invention may exhibit polymorphism, and that the invention encompasses all such forms which possess metalloproteinases inhibition activity and in particular TACE inhibition activity.

The present invention relates to compounds of the invention as defined herein as well as to the salts thereof. Salts for use in pharmaceutical compositions will be pharmaceutically acceptable salts, but other salts may be useful in the production of the compounds of the invention and their pharmaceutically acceptable salts. Pharmaceutically acceptable salts of the invention may, for example, include acid addition salts of compounds of the invention as defined herein which are sufficiently basic to form such salts. Such acid addition salts include but are not limited to hydrochloride, hydrobromide, citrate and maleate salts and salts formed with phosphoric and sulphuric acid. In addition where compounds of the invention are sufficiently acidic, salts are base salts and examples include but are not limited to, an alkali metal salt for example sodium or potassium, an alkaline earth metal salt for example calcium or magnesium, or organic amine salts for example triethylamine or tris-(2-hydroxyethyl)amine

The compounds of the invention may also be provided as in vivo hydrolysable esters. An in vivo hydrolysable ester of a compound of the invention containing a carboxy or hydroxy group is, for example a pharmaceutically acceptable ester which is cleaved in the human or animal body to produce the parent acid or alcohol. Such esters can be identified by administering, for example, intravenously to a test animal, the compound under test and subsequently examining the test animal's body fluid.

Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in compounds of this invention.

Suitable pharmaceutically-acceptable esters for hydroxy include inorganic esters such as phosphate esters (including phosphoramidic cyclic esters) and α-acyloxyalkyl ethers and related compounds which as a result of the in vivo hydrolysis of the ester breakdown to give the parent hydroxy group/s. Examples of α-acyloxyalkyl ethers include acetoxymethoxy and 2,2-dimethylpropionyloxymethoxy. A selection of in vivo hydrolysable ester forming groups for hydroxy include C₁₋₁₀alkanoyl, for example formyl, acetyl; benzoyl; phenylacetyl; substituted benzoyl and phenylacetyl, C₁₋₁₀alkoxycarbonyl (to give alkyl carbonate esters), for example ethoxycarbonyl; di-(C₁₋₄)alkylcarbamoyl and N-(di-(C₁₋₄)alkylaminoethyl)-N-(C₁₋₄)alkylcarbamoyl (to give carbamates); di-(C₁₋₄)alkylaminoacetyl and carboxyacetyl. Examples of ring substituents on phenylacetyl and benzoyl include aminomethyl, (C₁₋₄)alkylaminomethyl and di-((C₁₋₄)alkyl)aminomethyl, and morpholino or piperazino linked from a ring nitrogen atom via a methylene linking group to the 3- or 4-position of the benzoyl ring. Other interesting in vivo hydrolysable esters include, for example, R^{A}C(O)O(C₁₋₆)alkyl-CO—, wherein R^{A} is for example, benzyloxy-(C₁₋₄)alkyl, or phenyl). Suitable substituents on a phenyl group in such esters include, for example, 4-(C₁₋₄)piperazinyl-(C₁₋₄)alkyl, piperazinyl-(C₁₋₄)alkyl and morpholino-(C₁₋₄)alkyl.

In this specification the generic term “alkyl” includes both straight-chain and branched-chain alkyl groups. However references to individual alkyl groups such as “propyl” are specific for the straight chain version only and references to individual branched-chain alkyl groups such as tert-butyl are specific for the branched chain version only. For example, “C₁₋₃alkyl” includes methyl, ethyl, propyl and isopropyl, “C₁₋₄alkyl” additionally includes butyl and tert-butyl and examples of “C₁₋₆alkyl” include the examples of “C₁₋₄alkyl” and additionally pentyl, 2,3-dimethylpropyl, 3-methylbutyl and hexyl. An analogous convention applies to other generic terms, for example “C₂₋₄alkenyl” includes vinyl, allyl and 1-propenyl and examples of “C₂₋₆alkenyl” include the examples of “C₂₋₄alkenyl” and additionally 1-butenyl, 2-butenyl, 3-butenyl, 2-methylbut-2-enyl, 3-methylbut-1-enyl, 1-pentenyl, 3-pentenyl and 4-hexenyl. Examples of “C₂₋₄alkynyl” includes ethynyl, 1-propynyl, 2-propynyl, 3-butynyl and examples of “C₂₋₆alkynyl” include the examples of “C₂₋₄alkynyl” and additionally 2-pentynyl, hexynyl and 1-methylpent-2-ynyl. Where examples are given for generic terms, it should be noted that these examples are not limiting.

“Cycloalkyl” is a monocyclic, saturated alkyl ring. The term “C₃₋₄cycloalkyl” includes cyclopropyl and cyclobutyl. The term “C₃₋₅cycloalkyl” includes “C₃₋₄cycloalkyl and cyclopentyl. The term “C₃₋₆cycloalkyl” includes “C₃₋₅cycloalkyl”, and cyclohexyl. The term “C₃₋₇cycloalkyl” includes “C₃₋₆cycloalkyl” and additionally cycloheptyl. The term “C₃₋₁₀cycloalkyl” includes “C₃₋₇cycloalkyl” and additionally cyclooctyl, cyclononyl and cyclodecyl.

“Cycloalkenyl” is a monocyclic ring containing 1, 2, 3 or 4 double bonds. Examples of “C₅₋₆cycloalkenyl” are cyclopentenyl, cyclohexenyl and cyclohexadiene, “C₅₋₇cycloalkenyl” additionally includes cycloheptadiene and examples of “C₅₋₁₀cycloalkenyl” include the examples of “C₅₋₇cycloalkenyl” and cyclooctatriene.

Unless otherwise specified “aryl” is monocyclic or bicyclic. Examples of “aryl” therefore include phenyl (an example of monocyclic aryl) and naphthyl (an example of bicyclic aryl).

Examples of “arylC₁₋₄alkyl” are benzyl, phenethyl, naphthylmethyl and naphthylethyl.

Unless otherwise specified “heteroaryl” is a monocyclic or bicyclic aryl ring containing 5 to 10 ring atoms of which 1, 2, 3 or 4 ring atoms are chosen from nitrogen, sulphur or oxygen where a ring nitrogen or sulphur may be oxidised. Examples of heteroaryl are pyridyl, imidazolyl, quinolinyl, cinnolyl, pyrimidinyl, thienyl, pyrrolyl, pyrazolyl, thiazolyl, oxazolyl, isoxazolyl, pyrazinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl and pyrazolopyridinyl. Preferably heteroaryl is pyridyl, imidazolyl, quinolinyl, pyrimidinyl, thienyl, pyrazolyl, thiazolyl, oxazolyl and isoxazolyl. More preferably heteroaryl is pyridyl, imidazolyl and pyrimidinyl. Examples of “monocyclic heteroaryl” are pyridyl, imidazolyl, pyrimidinyl, thienyl, pyrrolyl, pyrazolyl, thiazolyl, oxazolyl, isoxazolyl and pyrazinyl. Examples of “bicyclic heteroaryl” are quinolinyl, quinazolinyl, cinnolinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl and pyrazolopyridinyl. Preferred examples of B when B is heteroaryl are those examples of bicyclic heteroaryl.

Examples of “heteroarylC₁₋₄alkyl” are pyridylmethyl, pyridylethyl, pyrimidinylethyl, pyrimidinylpropyl, pyrimidinylbutyl, imidazolylpropyl, imidazolylbutyl, quinolinylpropyl, 1,3,4-triazolylpropyl and oxazolylmethyl.

“Heterocyclyl” is a saturated, unsaturated or partially saturated, monocyclic or bicyclic ring (unless otherwise stated) containing 4 to 12 atoms of which 1, 2, 3 or 4 ring atoms are chosen from nitrogen, sulphur or oxygen, which may, unless otherwise specified, be carbon or nitrogen linked, wherein a —CH₂— group can optionally be replaced by a —C(O)—; and where unless stated to the contrary a ring nitrogen or sulphur atom is optionally oxidised to form the N-oxide or S-oxide(s); a ring —NH is optionally substituted by acetyl, formyl, methyl or mesyl; and a ring is optionally substituted by one or more halo. Examples and suitable values of the term “heterocyclyl” are piperidinyl, N-acetylpiperidinyl, N-methylpiperidinyl, N-formylpiperazinyl, N-mesylpiperazinyl, homopiperazinyl, piperazinyl, azetidinyl, oxetanyl, morpholinyl, tetrahydroisoquinolinyl, tetrahydroquinolinyl, indolinyl, pyranyl, dihydro-2H-pyranyl, tetrahydrofuranyl, 2,5-dioximidazolidinyl, 2,2-dimethyl-1,3-dioxolanyl and 3,4dimethylenedioxyphenyl. Preferred values are 3,4-dihydro-2H-pyran-5-yl, tetrahydrofuran-2-yl, 2,5-dioximidazolidinyl, 2,2-dimethyl-1,3-dioxolan-2-yl and 3,4-dimethylenedioxyphenyl. Other values are pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinoline, tetrahydroisoquinoline and isoindolinyl. Examples of monocyclic heterocyclyl are piperidinyl, N-acetylpiperidinyl, N-methylpiperidinyl, N-formylpiperazinyl, N-mesylpiperazinyl, homopiperazinyl, piperazinyl, azetidinyl, oxetanyl, morpholinyl, pyranyl, tetrahydrofuranyl, 2,5-dioximidazolidinyl and 2,2-dimethyl-1,3-dioxolanyl. Examples of bicyclic heterocyclyl are pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinolinyl, tetrahydroisoquinolinyl, isoindolinyl, 2,3-methylenedioxyphenyl, and 3,4-methylenedioxyphenyl. Examples of saturated heterocyclyl are piperidinyl, pyrrolidinyl and morpholinyl.

The term “halo” refers to fluoro, chloro, bromo and iodo.

Examples of “C₁₋₃alkoxy” and “C₁₋₄alkoxy” include methoxy, ethoxy, propoxy and isopropoxy. Examples of “C₁₋₆alkoxy” include the examples of “C₁₋₄alkoxy” and additionally pentyloxy, 1-ethylpropoxy and hexyloxy.

“Heteroalkyl” is alkyl containing at least one carbon atom and having at least one carbon atom replaced by a hetero group independently selected from N, O, S, SO, SO₂, (a hetero group being a hetero atom or group of atoms). Examples include —OCH₂—, CH₂O—, —CH₂SO₂CH₂CH₂— and —OCH(CH₃)—.

“HaloC₁₋₄alkyl” is a C₁₋₄alkyl group substituted by one or more halo. Examples of “haloC₁₋₄alkyl” include fluoromethyl, trifluoromethyl, 1-chloroethyl, 2-chloroethyl, 2-bromopropyl, 1-fluoroisopropyl and 4-chlorobutyl. Examples of “haloC₁₋₆alkyl” include the examples of “haloC₁₋₄alkyl” and 1-chloropentyl, 3-chloropentyl and 2-fluorohexyl.

Examples of “hydroxyC₁₋₄alkyl” include hydroxymethyl, 1-hydroxyethyl, 2-hydroxyethyl, 2-hydroxypropyl, 1-hydroxyisopropyl and 4-hydroxybutyl.

Example of “C₁₋₄alkoxyC₁₋₄alkyl” include methoxymethyl, ethoxymethyl, methoxyethyl, methoxypropyl and propoxybutyl.

“HaloC₁₋₄alkoxyC₁₋₄alkyl” is a C₁₋₄alkoxyC₁₋₄alkyl group substituted by one or more halo. Examples of “haloC₁₋₄alkoxyC₁₋₄alkyl” include 1-(chloromethoxy)ethyl, 2-fluoroethoxymethyl, trifluoromethylmethoxy, 2-(4-bromobutoxy)ethyl and 2-(2-iodoethoxy)ethyl.

Examples of “carboxyC₁₋₄alkyl” include carboxymethyl, 2-carboxyethyl and 2-carboxypropyl.

Heterocyclic rings are rings containing 1, 2 or 3 ring atoms selected from nitrogen, oxygen and sulphur. “Heterocyclic 5- to 7-membered” rings are pyrrolidinyl, piperidinyl, piperazinyl, homopiperidinyl, homopiperazinyl, thiomorpholinyl, thiopyranyl and morpholinyl. “Heterocyclic 4- to 7-membered” rings include the examples of “heterocyclic 5 to 7-membered” and additionally azetidinyl. Similarly “heterocyclic 5- to 6-membered” rings includes pyrrolidinyl, pyrrolyl, pyrimidinyl, pyridinyl and piperidinyl, and “heterocyclic 4- to 6-membered” rings additionally includes azetidinyl.

Carbocyclic rings are saturated, partially saturated of unsaturated rings containing only carbon ring atoms. Examples are cyclopentanyl, cyclohexanyl, cyclohexenyl and phenyl. Such rings may be optionally substituted by halo, C₁₋₄alkoxy, haloC₁₋₄alkyl or C₁₋₄alkoxyC₁₋₄alkyl.

Saturated 5 to 7-membered rings include cyclopentane, cyclohexane and cycloheptane and saturated 3 to 7-membered rings additionally include cyclopropane and cyclobutane. Saturated 5 to 7-membered rings and 3 to 7-membered rings optionally containing 1 or 2 heteroatom groups selected from NH, O, S, SO and SO₂ include pyrrolidine, piperidine, tetrahydrofuran and tetrahydropyran.

Where optional substituents are chosen from “one of more” groups or substituents it is to be understood that this definition includes all substituents being chosen from one of the specified groups or the substituents being chosen from two or more of the specified groups. Preferably “one or more” means “1, 2 or 3” and this is particularly the case when the group or substituent is halo. “One or more” may also mean “1 or 2”.

Compounds of the present invention have been named with the aid of computer software (ACD/Name version 5.09).

Preferred values of Y¹, Y², z, n, t, R⁴, R⁵, R⁶, R⁷, R¹² and R¹³ for a compound of formula (I), (IA) or (IB) are as follows. Such values may be used where appropriate with any of the definitions, claims or embodiments defined herein.

In one aspect of the invention Y¹ and Y² are both O.

In one aspect of the invention z is NR⁸.

In one aspect of the invention n is 1. In another aspect n is 0.

In one aspect of the invention t is 0. In another aspect t is 1.

In one aspect of the invention R⁴ is hydrogen or methyl. In another aspect R⁴ is hydrogen.

In one aspect of the invention R⁵ is hydrogen or methyl. In another aspect R⁵ is hydrogen.

In one aspect of the invention R⁶ is hydrogen or methyl. In another aspect R⁶ is hydrogen.

In one aspect of the invention R⁷ is hydrogen or a group selected from C₁₋₆alkyl, C₃₋₇cycloalkyl, aryl, heteroaryl or heterocyclyl where the group is optionally substituted by heterocyclyl, aryl and heteroaryl; and wherein the group from which R⁷ may be selected is optionally substituted on the group and/or on its optional substituent by one or more substituents independently selected from halo, cyano, C₁₋₄alkyl, —OR²¹, —CO₂R²¹, —NR²¹COR²², —NR²¹CO₂R²² and —CONR²¹R²². In another aspect R⁷ is hydrogen or a group selected from C₁₋₄alkyl, arylC₁₋₄alkyl, heteroarylC₁₋₄alkyl, heterocyclylC₁₋₄alkyl, aryl, heteroaryl, heterocyclyl and C₃₋₅cycloalkyl which group is optionally substituted by cyano, C₁₋₄alkyl, —COC₁₋₄alkyl, halo, —OR²¹, —NR²¹R²², —CO₂R²¹ and —NR²¹CO₂R²². In another aspect R⁷ is hydrogen or a group selected from C₁₋₄alkyl, tetrahydrofuranyl, tetrahydropyranyl, pyrrolidinyl, piperidinyl, morpholinyl optionally substituted by one or more C₁₋₄alkoxy, fluoro, —COC₁₋₃alkyl or —SO₂C₁₋₃alkyl. In a further aspect R⁷ is selected from hydrogen, methyl, ethyl, propyl, cyclopropyl, isopropyl, butyl, tert-butyl, isobutyl, 1-hydroxyethyl, 2-hydroxyethyl, 3-hydroxypropyl, methoxymethyl, 2-methoxyethyl, aminomethyl, 2-aminoethyl, 2-cyanoethyl, phenyl, pyridyl, benzyl, 3-methylbenzyl, phenylethyl, 4-chlorophenylethyl, 4-fluorophenylethyl, phenylpropyl, 4-chlorophenylpropyl, 4-fluorophenylpropyl, 4-methylpiperazin-1-ylethyl, morpholin-4-ylpropyl, pyrimidin-2-ylethyl, pyrimidin-2-ylpropyl, pyrimidin-2-ylbutyl, 5-fluoropyrimidin-2-ylpropyl, imidazol-1-ylpropyl, imidazol-1-ylbutyl, 1,3,4-triazolylpropyl, piperidinyl, carbamoylphenyl, tetrahydro-2H-pyranyl, tetrahydro-2H-pyranylmethyl, pyrid-2-ylmethyl, pyrid-4-ylmethyl, pyrid-3-ylmethyl, piperidin-4-ylmethyl, N-(tert-butoxycarbonyl)piperidin-4-yl, N-(methylcarbonyl)piperidin-4-yl, N-(tert-butoxycarbonyl)aminomethyl, benzyloxyethyl, N-(tert-butoxycarbonyl)piperidin-4-ylmethyl, (3,4,4-trimethyl-2,5-dioximidazolidin-1-yl)methyl, and N-benzoyl-N-phenylaminomethyl. In a further aspect R⁷ is hydrogen or C₁₋₄alkyl optionally substituted by halo, hydroxy, C₁₋₄alkoxy or amino. In another aspect R⁷ is hydrogen or C₁₋₄alkyl. In a further aspect R⁷ is hydrogen, methyl or ethyl.

In one aspect of the invention R⁸ is hydrogen or methyl. In another aspect R⁸ is hydrogen.

In one aspect of the invention R⁹ is hydrogen or methyl.

In one aspect of the invention R¹⁰ is hydrogen or methyl.

In one aspect of the invention R¹¹ is methyl.

In one aspect of the invention R¹² is hydrogen or methyl. In another aspect R¹² is hydrogen.

In one aspect of the invention R¹³ is hydrogen or methyl. In another aspect R¹³ is hydrogen.

In one aspect of the invention R¹⁶ is hydrogen or methyl.

In one aspect of the invention R¹⁷ is selected from fluoro, chloro, methyl or methoxy.

In one aspect of the invention R¹⁸ is hydrogen or a group selected from C₁₋₆alkyl, aryl and arylC₁₋₄alkyl where the group is optionally substituted by halo. In another aspect R¹⁸ is hydrogen or a group selected from methyl, phenyl and benzyl where the group is optionally substituted by chloro.

In one aspect of the invention R¹⁹ is a group selected from C₁₋₆alkyl, aryl and arylC₁₋₄alkyl where the group is optionally substituted by halo. In another aspect R¹⁹ is a group selected from methyl, phenyl and benzyl where the group is optionally substituted by chloro. In one aspect R¹⁹ is methyl.

In one aspect of the invention R²⁰ is hydrogen or methyl.

In one aspect of the invention R²¹ is hydrogen, methyl, ethyl, phenyl and benzyl.

In one aspect of the invention R²² is hydrogen, methyl, ethyl, tert-butyl, phenyl and benzyl. In another aspect R²² is hydrogen or methyl.

In one aspect of the invention R²⁵ is a group selected from C₁₋₆alkyl, aryl and arylC₁₋₄alkyl where the group is optionally substituted by halo. In another aspect R²⁵ is a group selected from methyl, phenyl and benzyl where the group is optionally substituted by chloro. In one aspect R²⁵ is methyl.

Preferred values of W, V, B, R³, R⁴, R⁵, R⁶ and R⁷ for a compound of formula (I) are as follows:

In one aspect of the invention W is NR¹. In another aspect W is CR¹R². In a further aspect W is a bond.

In one aspect of the invention V is C═O. In another aspect V is SO₂. In a further aspect V is NR¹⁵C═O.

In one aspect of the invention V and W together form C═O. In another aspect V and W together form NR¹⁵C═ONR¹.

In one aspect of the invention, when V is C(═O), NR¹⁵C(═O) or NR¹⁵SO₂; or when V is SO₂ and n is 1 and W is NR¹, CR¹R² or a bond; or when V is SO₂ and n is 0 and W is CR¹R²; then B is a group selected from aryl, heteroaryl and heterocyclyl where each group is optionally substituted by one or more groups independently selected from nitro, trifluoromethyl, trifluoromethoxy, halo, C₁₋₄alkyl (optionally substituted by one or more halo), C₂₋₄alkynyl, heteroaryl, —OR⁹, cyano, —NR⁹R¹⁰, —CONR⁹R¹⁰ and —NR⁹COR¹⁰; or B is C₂₋₄alkenyl or C₂₋₄alkynyl optionally substituted by C₁₋₄alkyl, C₃₋₆cycloalkyl or heterocyclyl. In one aspect of the invention B is a group selected from aryl and heteroaryl where each group is optionally substituted by one or more groups independently selected from halo, C₁₋₄alkyl (optionally substituted by one or more halo), C₂₋₄alkenyl (optionally substituted by halo) and C₂₋₄alkynyl (optionally substituted by halo); or B is C₂₋₄alkenyl or C₂₋₄alkynyl, each being optionally substituted by a group selected from C₁₋₄alkyl, C₃₋₆cycloalkyl, aryl, heteroaryl, heterocyclyl whereby this group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl and C₁₋₄alkoxy; provided that when t is 0 and B is monocyclic aryl or monocyclic heteroaryl then the monocyclic group that is B is substituted on the carbon or nitrogen adjacent to the atom to which the oxygen is attached, by a substituent group described above. In one aspect of the invention, when V is SO₂ and n is 0 and W is NR¹ or a bond; B is a group selected from bicyclic aryl, bicyclic heteroaryl and bicyclic heterocyclyl, where each group is optionally substituted by one or more groups independently selected from nitro, trifluoromethyl, trifluoromethoxy, halo, C₁₋₄alkyl (optionally substituted by one or more halo), C₂₋₄alkynyl, heteroaryl, —OR⁹, cyano, —NR⁹R¹⁰, —CONR⁹R¹⁰ and —NR⁹COR¹⁰; or B is C₂₋₄alkenyl or C₂₋₄alkynyl optionally substituted by C₁₋₄alkyl, C₃₋₆cycloalkyl or heterocyclyl. In a further aspect of the invention B is 2-methylquinolin-4-yl, 2,5-dimethylphenyl or 2,5-dimethylpyrid-4-yl.

In one aspect of the invention R¹ is hydrogen or methyl.

In one aspect of the invention R² is hydrogen or methyl.

In one aspect of the invention R³ is hydrogen or methyl.

In one aspect of the invention R¹ and R³ together with the nitrogen or carbon and carbon to which they are respectively attached form a 2,2-dimethylthiomorpholine, piperidine, pyrrolidine, piperazine, morpholine, cyclopentane or cyclohexane ring.

In one aspect of the invention R³ and R⁴ together form a pyrrolidine ring or a tetrahydro-2H-pyran ring.

In one aspect of the invention R³ and R⁵ together with the carbon atoms to which they are attached form a piperidine ring substituted by methyl.

In one aspect of the invention R³ and R⁷ together with the carbon atoms to which they are each attached and (CR⁵R⁶)ₙ form a piperidinyl, pyrrolidinyl, piperazine or morpholine ring.

In one aspect R¹⁵ is hydrogen or methyl.

In addition to the preferred values of Y¹, Y², z, n, t, R⁴, R⁵, R⁶, R⁷, R¹² and R¹³ mentioned above in relation to a compound of formula (I), (IA) or (IB), other preferred values of W, V, B, R³, R⁴, R⁵ and R⁷ for a compound of formula (IA) are as follows. These values may also be used where appropriate with any of the definitions, claims or embodiments defined herein.

In one aspect of the invention W is a bond or CR¹R². In another aspect W is NR¹. In another aspect W is CR¹R². In a further aspect W is a bond.

In this aspect of the invention V is NR¹⁵SO₂.

In one aspect of the invention, B is a group selected from aryl, heteroaryl and heterocyclyl where each group is optionally substituted by one or more groups independently selected from nitro, trifluoromethyl, trifluoromethoxy, halo, C₁₋₄alkyl (optionally substituted by one or more halo), C₂₋₄alkynyl, heteroaryl, —OR⁹, cyano, —NR⁹R¹⁰, —CONR⁹R¹⁰ and —NR⁹COR¹⁰; or B is C₂₋₄alkenyl or C₂₋₄alkynyl optionally substituted by C₁₋₄alkyl, C₃₋₆cycloalkyl or heterocyclyl. In another aspect, B is phenyl, naphthyl, pyridyl, imidazolyl, quinolinyl, cinnolyl, isoquinolinyl, thienopyridyl, naphthyridinyl, 2,5-methylenedioxyphenyl, 3,4-methylenedioxyphenyl, thienopyrimidinyl, pyrimidinyl, thienyl, pyrrolyl, pyrazolyl, thiazolyl, oxazolyl, isoxazolyl, pyrazinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinolinyl, tetrahydroisoquinolinyl and isoindolinyl, where each is optionally substituted by one or more groups independently selected from nitro, trifluoromethyl, trifluoromethoxy, halo, C₁₋₄alkyl (optionally substituted by one or more fluoro), C₂₋₄alkynyl, heteroaryl, —OR⁹, cyano, —NR⁹R¹⁰, —CONR⁹R¹⁰ and —NR⁹COR¹⁰; or B is vinyl or ethynyl optionally substituted by C₁₋₄alkyl. In a preferred aspect B is bicyclic aryl, bicyclic heteroaryl or bicyclic heterocyclyl optionally substituted by one or more groups independently selected from nitro, trifluoromethyl, trifluoromethoxy, halo, cyano, C₁₋₄alkyl (optionally substituted by R⁹ or C₁₋₄alkoxy, or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is phenyl, pyridyl or pyrimidinyl substituted at the 2- and 5 positions (whereby the 1-position is the atom by which B is bonded to (CR¹²CR¹³)ₜ) by groups independently selected from nitro, trifluoromethyl, trifluoromethoxy, halo, cyano, C₁₋₄alkyl (optionally substituted by R⁹ or C₁₋₄alkoxy, or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰. In a preferred aspect B is bicyclic aryl, bicyclic heteroaryl or bicyclic heterocyclyl optionally substituted by one or more groups independently selected from nitro, trifluoromethyl, trifluoromethoxy, halo, cyano, C₁₋₄alkyl (optionally substituted by R⁹ or C₁₋₄alkoxy, or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰. In a further aspect B is quinolin-4-yl, naphth-1-yl, 2-methylquinolin-4-yl, 3-methylnaphthyl, 7-methylquinolin-5-yl, 6-methylquinolin-8-yl, 7-methylisoquinolin-5-yl, 6-methylthieno[2,3-b]pyridyl, 5-methylthieno[3,2-b]pyridyl, 2-methyl-1,8-naphthyridinyl, 2-trifluoromethylquinolin-4-yl, 2-ethynylquinolin-4-yl, 7-chloroquinolin-5-yl, 7-fluoro-2-methylquinolin-4-yl, 2-methyl-N-oxoquinolin-4-yl, 3-methylisoquinolin-1-yl, 5-fluoro-2-methylquinolin-4-yl, 2,5-dimethylpyridin-4-yl, 2,5-dimethylphenyl, 2,5-difluorophenyl, 2,3-methylenedioxyphenyl, 3,4-methylenedioxyphenyl, 5-fluoro-2-methylpyridinyl, 1-methylquinolinyl, 7-chloroquinolin-4-yl, 8-chloroquinolin-4-yl, 6-chloroquinolin-4-yl, 5-methylthieno[2,3-d]pyrimidin-4-yl, 7-methylthieno[3,2-d]pyrimidin-4-yl, 8-fluoroquinolin-4-yl, 6-fluoroquinolin-4-yl, 2-methylquinolin-4-yl, 6-chloro-2-methylquinolin-4-yl, 1,6-naphthyridin-4-yl, thieno[3,2-b]pyrid-7-yl, 5-fluoro-2-(isoxazol-5-yl)phenyl, 2-chloro-5-fluorophenyl, vinyl, ethynyl, prop-1-enyl, prop-1-ynyl or but-1-ynyl. In another aspect of the invention B is a group selected from aryl and heteroaryl where each group is optionally substituted by one or more groups independently selected from halo, C₁₋₄alkyl (optionally substituted by one or more halo), C₂₋₄alkenyl (optionally substituted by halo) and C₂₋₄alkynyl (optionally substituted by halo); or B is C₂₋₄alkenyl or C₂₋₄alkynyl, each being optionally substituted by a group selected from C₁₋₄alkyl, C₃₋₆cycloalkyl, aryl, heteroaryl, heterocyclyl which group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl and C₁₋₄alkoxy; provided that when t is 0 and B is monocyclic aryl or monocyclic heteroaryl then the monocyclic group that is B is substituted on the carbon or nitrogen adjacent to the atom to which the oxygen is attached, by a substituent group described above. In another aspect of the invention B is a group selected from quinolinyl, pyridyl and phenyl where each group is optionally substituted by one or more methyl, trifluoromethyl, trifluoromethoxy, or halo. In another aspect B is C₂₋₄alkenyl or C₂₋₄alkynyl optionally substituted by C₁₋₄alkyl, C₃₋₆cycloalkyl or heterocyclyl. In a further aspect of the invention B is 2-methylquinolin-4-yl, 2,5-dimethylphenyl or 2,5-dimethylpyrid-4-yl. In yet another aspect B is 2-methylquinolin-4-yl or 2,5-dimethylphenyl. In a further aspect B is 2-methylquinolin-4-yl.

In one aspect of the invention R¹ is hydrogen or C₁₋₄alkyl optionally substituted by halo, hydroxy or C₁₋₄alkoxy. In another aspect R¹ is hydrogen or methyl.

In one aspect of the invention R² is hydrogen or methyl.

In one aspect of the invention R³ is hydrogen, methyl, ethyl, propyl or phenyl. In another aspect R³ is hydrogen.

In one aspect of the invention R¹ and R³ together with the nitrogen or carbon atoms and carbon atom to which they are respectively attached form a 2,2-dimethylthiomorpholine, piperidine, pyrrolidine, piperazine, morpholine, cyclopentane or cyclohexane ring.

In one aspect of the invention R³ and R⁴ together with the carbon atom to which they are attached form a piperidine, pyrrolidine, tetrahydrofuran or tetrahydropyran ring. In one aspect of the invention R³ and R⁴ together form a pyrrolidine ring or a tetrahydro-2H-pyran ring.

In one aspect of the invention R³ and R⁵ together with the carbon atoms to which they are attached form a piperidine or pyrrolidine ring optionally substituted by methyl. In another aspect R³ and R⁵ together with the carbon atoms to which they are attached form a piperidine ring substituted by methyl

In one aspect of the invention R³ and R⁷ together with the carbon atoms to which they are each attached and (CR⁵R⁶)ₙ form a piperidine, pyrrolidine, piperazine, morpholine, tetrahydrofuran, tetrahydrpyran, cyclohexane or cyclopentane ring. In another aspect R³ and R⁷ together with the carbon atoms to which they are each attached and (CR⁵R⁶)ₙ form a piperidinyl, pyrrolidinyl, piperazine or morpholine ring. In a further aspect R³ and R⁷ together with the carbon atoms to which they are each attached and (CR⁵R⁶)ₙ form a tetrahydrofuran, cylohexane or cyclopentane ring.

In one aspect R¹⁵ is hydrogen or methyl.

In addition to the preferred values of Y¹, Y², z, n, t, R⁴, R⁵, R⁶, R⁷, R¹² and R¹³ mentioned above in relation to a compound of formula (I), (IA) or (IB), other preferred values of W, V, B, R³, R⁴, R⁵ and R⁷ for a compound of formula (IB) are as follows. These values may also be used where appropriate with any of the definitions, claims or embodiments defined hereinbefore or hereinafter.

In one aspect of the invention W is NR¹.

In one aspect of the invention V is SO₂. In another aspect V is CO.

In one aspect of the invention, B is a group selected from aryl, heteroaryl and heterocyclyl where each group is optionally substituted by one or more groups independently selected from nitro, trifluoromethyl, trifluoromethoxy, halo, C₁₋₄alkyl (optionally substituted by one or more halo), C₂₋₄alkynyl, heteroaryl, —OR⁹, cyano, —NR⁹R¹⁰, —CONR⁹R¹⁰ and —NR⁹COR¹⁰; or B is C₂₋₄alkenyl or C₂₋₄alkynyl optionally substituted by C₁₋₄alkyl, C₃₋₆cycloalkyl or heterocyclyl; provided that when t is 0 such that B is directly attached to the oxygen atom shown in formula (IB) and B is monocyclic aryl or monocyclic heteroaryl and n is 0 then the monocyclic group that is B is substituted on one of the atoms adjacent to the atom to which the oxygen is attached, by a group selected from those listed in the definition of B which optionally substitute B. In one aspect of the invention B is a group selected from aryl and heteroaryl where each group is optionally substituted by one or more groups independently selected from halo, C₁₋₄alkyl (optionally substituted by one or more halo), C₂₋₄alkenyl (optionally substituted by halo) and C₂₋₄alkynyl (optionally substituted by halo); or B is C₂₋₄alkenyl or C₂₋₄alkynyl, each being optionally substituted by a group selected from C₁₋₄alkyl, C₃₋₆cycloalkyl, aryl, heteroaryl, heterocyclyl whereby this group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰,
—NR⁹SO₂R¹¹, C₁₋₄alkyl and C₁₋₄alkoxy; provided that when t is 0 and B is monocyclic aryl or monocyclic heteroaryl then the monocyclic group that is B is substituted on the carbon atom adjacent to the atom to which the oxygen is attached, by a substituent group described above. In another aspect, B is phenyl, naphthyl, pyridyl, imidazolyl, quinolinyl, cinnolyl, isoquinolinyl, thienopyridyl, naphthyridinyl, 2,5-methylenedioxyphenyl, 3,4-methylenedioxyphenyl, thienopyrimidinyl, pyrimidinyl, thienyl, pyrrolyl, pyrazolyl, thiazolyl, oxazolyl, isoxazolyl, pyrazinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinolinyl, tetrahydroisoquinolinyl and isoindolinyl, where each is optionally substituted by one or more groups independently selected from nitro, trifluoromethyl, trifluoromethoxy, halo, C₁₋₄alkyl (optionally substituted by one or more fluoro), C₂₋₄alkynyl, heteroaryl, —OR⁹, cyano, —NR⁹R¹⁰, —CONR⁹R¹⁰ and —NR⁹COR¹⁰; or B is vinyl or ethynyl optionally substituted by C₁₋₄alkyl, provided that t is 1. In a further aspect B is quinolin-4-yl, naphthyl, 2-methylquinolin-4-yl, 3-methylnaphthyl, 7-methylquinolin-5-yl, 6-methylquinolin-8-yl, 7-methylisoquinolin-5-yl, 6-methylthieno[2,3-b]pyridyl, 5-methylthieno[3,2-b]pyridyl, 2-methyl-1,8-naphthyridinyl, 2-trifluoromethylquinolin-4-yl, 2-ethynylquinolin-4-yl, 7-chloroquinolin-5-yl, 7-fluoro-2-methylquinolin-4-yl, 2-methyl-N-oxoquinolin-4-yl, 3-methylisoquinolin-1-yl, 5-fluoro-2-methylquinolin-4-yl, 2,5-dimethylpyridin-4-yl, 2,5-dimethylphenyl, 2,5-difluorophenyl, 5-fluoro-2-methylphenyl, 2,3-methylenedioxyphenyl, 3,4-methylenedioxyphenyl, 5-fluoro-2-methylpyridinyl, 1-methylquinolinyl, 7-chloroquinolin-4-yl, 8-chloroquinolin-4-yl, 6-chloroquinolin-4-yl, 5-methylthieno[2,3-d]pyrimidin-4-yl, 7-methylthieno[3,2-d]pyrimidin-4-yl, 8-fluoroquinolin-4-yl, 6-fluoroquinolin-4-yl, 2-methylquinolin-4-yl, 6-chloro-2-methylquinolin-4-yl, 1,6-naphthyridin-4-yl, thieno[3,2-b]pyrid-7-yl, 2-chloro-5-fluorophenyl, vinyl, ethynyl, prop-1-enyl, prop-1-ynyl or but-1-ynyl. In one aspect B is a group selected from bicyclic aryl, bicyclic heteroaryl and bicyclic heterocyclyl, where each group is optionally substituted by one or more groups independently selected from nitro, trifluoromethyl, trifluoromethoxy, halo, cyano, C₁₋₄alkyl (optionally substituted by R⁹ or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl, heterocyclyl which group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl or C₁₋₄alkoxy. In another aspect B is quinolin-4-yl, naphthyl, 2-methylquinolin-4-yl, 3-methylnaphthyl, 7-methylquinolin-5-yl, 6-methylquinolin-8-yl, 7-methylisoquinolin-5-yl, 6-methylthieno[2,3-b]pyridyl, 5-methylthieno[3,2-b]pyridyl, 2-methyl-1,8-naphthyridinyl, 2-trifluoromethylquinolin-4-yl, 2-ethynylquinolin-4-yl, 7-chloroquinolin-5-yl, 7-fluoro-2-methylquinolin-4-yl, 2-methyl-N-oxoquinolin-4-yl, 3-methylisoquinolin-1-yl, 5-fluoro-2-methylquinolin-4-yl, 2,3-methylenedioxyphenyl, 3,4-methylenedioxyphenyl, 1methylquinolinyl, 7-chloroquinolin-4-yl, 8-chloroquinolin-4-yl, 6-chloroquinolin-4-yl, 5-methylthieno[2,3-d]pyrimidin-4-yl, 7-methylthieno[3,2-d]pyrimidin-4-yl, 8-fluoroquinolin-4-yl, 6-fluoroquinolin-4-yl, 2-methylquinolin-4-yl, 6-chloro-2-methylquinolin-4-yl, 1,6-naphthyridin-4-yl, thieno[3,2-b]pyrid-7-yl, vinyl, ethynyl, prop-1-enyl, prop-1-ynyl or but-1-ynyl. In another aspect of the invention B is a group selected from quinolinyl, pyridyl and phenyl where each group is optionally substituted by one or more methyl, trifluoromethyl, trifluoromethoxy, halo or isoxazolyl provided that when n is 0 and t is 0, pyridyl or phenyl are substituted in the carbon atom adjacent to the atom to which the oxygen is attached. In a further aspect of the invention B is 2-methylquinolin-4-yl, 2,5-dimethylphenyl or 2,5-dimethylpyrid-4-yl. In yet another aspect B is 2-methylquinolin-4-yl.

In one aspect of the invention R¹ and R³ together with the nitrogen and carbon atoms to which they are respectively attached form a saturated 4- to 6-membered ring optionally containing a further heteroatom group selected from NH, O, S or SO₂. In another aspect R¹ and R³ together with the nitrogen and carbon atoms to which they are respectively attached form a saturated 5- to 6-membered ring optionally substituted on carbon by C₁₋₄alkyl, fluoro or C₁₋₄alkoxy. In another aspect R¹ and R³ together with the nitrogen and carbon atoms to which they are respectively attached form a saturated 5- to 6-membered ring i.e pyrrolidinyl or piperidinyl.

A preferred class of compound is of formula (IA) wherein:
Y¹ and Y² are both O;
z is NR⁸;
n is 0 or 1;
W is NR¹, CR¹R² or a bond;
V is NR⁵C(═O);
t is 1;
B is a group selected from aryl, heteroaryl and heterocyclyl where each group is optionally substituted by one or more groups independently selected from nitro, trifluoromethyl, trifluoromethoxy, halo, cyano, C₁₋₄alkyl (optionally substituted by R⁹ or C₁₋₄alkoxy one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR¹¹, —SOR¹¹, —SO₂R¹¹, —SO₂NR⁹R¹¹, —NR⁹SO₂R¹⁰, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl and heterocyclyl whereby this group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl or C₁₋₄alkoxy;
R¹ and R² are independently hydrogen or methyl;
R³ is hydrogen, methyl, ethyl, propyl or phenyl;
R⁴, R⁵, R⁶, R⁸, R⁹, R¹⁰, R¹², R¹³ and R¹⁵ are independently hydrogen or methyl;
R¹¹ is methyl;
R⁷ is hydrogen or a group selected from C₁₋₆alkyl, C₃₋₇cycloalkyl, aryl, heteroaryl or heterocyclyl which group is optionally substituted by heterocyclyl, aryl and heteroaryl; and wherein the group from which R⁷ may be selected is optionally substituted on the group and/or on its optional substituent by one or more substitutents independently selected from halo, cyano, C₁₋₄alkyl, —COC₁₋₄alkyl, —OR²¹, —NR²¹R²², —CO₂R²¹, —NR²¹COR²², —NR²¹CO₂R²² and —CONR²¹R²²;
or R³ and R⁷ together with the carbon atoms to which they are each attached and (CR⁵R⁶)ₙ form a piperidine, pyrrolidine, piperazine, morpholine, tetrahydrofuran, tetrahydropyran, cyclohexane or cyclopentane ring;
R²¹ is hydrogen, methyl, ethyl, phenyl or benzyl
R²² is hydrogen, methyl, ethyl, tert-butyl, phenyl or benzyl.

Another preferred class of compound is of formula (A) wherein:
Y¹ and Y² are both O;
z is NR⁸;
n is 0 or 1;
W is NR¹, CR¹R² or a bond;
V is NR¹⁵SO₂;
t is 1;
B is phenyl, naphthyl, pyridyl, imidazolyl, quinolinyl, cinnolyl, isoquinolinyl, thienopyridyl, naphthyridinyl, 2,5-methylenedioxyphenyl, 3,4-methylenedioxyphenyl, thienopyrimidinyl, pyrimidinyl, thienyl, pyrrolyl, pyrazolyl, thiazolyl, oxazolyl, isoxazolyl, pyrazinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinolinyl, tetrahydroisoquinolinyl and isoindolinyl, where each is optionally substituted by one or more groups independently selected from nitro, trifluoromethyl, trifluoromethoxy, halo, C₁₋₄alkyl (optionally substituted by one or more fluoro), C₂₋₄alkynyl, heteroaryl, —OR⁹, cyano, —NR⁹R¹⁰, —CONR⁹R¹⁰ and —NR⁹COR¹⁰; or B is vinyl or ethynyl optionally substituted by C₁₋₄alkyl;
R¹ and R² are independently hydrogen or methyl;
R³ is hydrogen, methyl, ethyl, propyl or phenyl;
R⁴, R⁵, R⁶, R⁸, R⁹, R¹⁰, R¹², R¹³ and R¹⁵ are independently hydrogen or methyl;
R⁷ is hydrogen or a group selected from C₁₋₄alkyl, arylC₁₋₄alkyl, heteroarylC₁₋₄alkyl, heterocyclylC₁₋₄alkyl, aryl, heteroaryl, heterocyclyl and C₃₋₅cycloalkyl where the group is optionally substituted by cyano, C₁₋₄alkyl, halo, —OR²¹, —CO₂R²¹ and —NR²¹CO₂R²²;
or R³ and R⁷ together with the carbon atoms to which they are each attached and (CR⁵R⁶)ₙ form a piperidine, pyrrolidine, piperazine, morpholine, tetrahydrofuran, tetrahydropyran, cyclohexane or cyclopentane ring;
R²¹ is hydrogen, methyl, ethyl, phenyl or benzyl.
R²² is hydrogen, methyl, ethyl, tert-butyl, phenyl or benzyl.

Another preferred class of compound is of formula (IA) wherein:
Y¹ and Y² are both O;
z is NR⁸;
n is 0 or 1;
W is NR¹, CR¹R² or a bond;
V is NR¹⁵SO₂;
t is 1;
B is bicyclic aryl, bicyclic heteroaryl or bicyclic heterocyclyl optionally substituted by one or more groups independently selected from nitro, trifluoromethyl, trifluoromethoxy, halo, cyano, C₁₋₄alkyl (optionally substituted by R⁹ or C₁₋₄alkoxy, or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR¹¹, —SOR¹¹, —SO₂R, —SO²NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is phenyl, pyridyl or pyrimidinyl substituted at the 2- and 5 positions (whereby the 1-position is the atom by which B is bonded to (CR¹²CR¹³)ₜ) by groups independently selected from nitro, trifluoromethyl, trifluoromethoxy, halo, cyano, C₁₋₄alkyl (optionally substituted by R⁹ or C₁₋₄alkoxy, or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰;
R¹ and R² are independently hydrogen or methyl;
R³ is hydrogen, methyl, ethyl, propyl or phenyl;
R⁴, R⁵, R⁶, R⁸, R⁹, R¹⁰, R¹², R¹³ and R¹⁵ are independently hydrogen or methyl;
R¹¹ is methyl;
R⁷ is hydrogen or C₁₋₄alkyl optionally substituted by halo, hydroxyl, C₁₋₄alkoxy or amino;
or R³ and R⁷ together with the carbon atoms to which they are each attached and (CR⁵R⁶)ₙ form a tetrahydrofuran, cyclohexane or cyclopentane ring.

Another preferred class of compound is of formula (IA) wherein:
Y¹ and Y² are both O;
z is NR⁸;
n is 0 or 1;
W is NR¹, CR¹R² or a bond;
V is NR¹⁵SO₂;
t is 1;
B is a group selected from quinolinyl, pyridyl and phenyl where each group is optionally substituted by 1 or 2 methyl, trifluoromethyl, trifluoromethoxy or halo;
R¹ and R² are independently hydrogen or methyl;
R³ is hydrogen, methyl, ethyl, propyl or phenyl;
R⁴, R⁵, R⁶, R⁸, R¹², R¹³ and R¹⁵ are independently hydrogen or methyl;
R⁷ is hydrogen, methyl or ethyl;
or R³ and R⁷ together with the carbon atoms to which they are each attached and (CR⁵R⁶)ₙ form a tetrahydrofuran, cyclohexane or cyclopentane ring;

A preferred class of compound is of the formula (IB) wherein:
Y¹ and Y² are both O;
z is NR⁸;
n is 0 or 1;
W is NR¹;
V is SO₂;
t is 0 or 1;
B is a group selected from aryl, heteroaryl and heterocyclyl where each group is optionally substituted by one or more groups independently selected from nitro, trifluoromethyl, trifluoromethoxy, halo, C₁₋₄alkyl (optionally substituted by one or more halo), C₂₋₄alkynyl, heteroaryl, —OR⁹, cyano, —NR⁹R¹⁰, —CONR⁹R¹⁰ and —NR⁹COR¹⁰; or B is C₂₋₄alkenyl or C₂₋₄alkynyl optionally substituted by C₁₋₄alkyl, C₃₋₆cycloalkyl or heterocyclyl; provided that when t is 0 such that B is directly attached to the oxygen atom shown in formula (IB) and B is monocyclic aryl or monocyclic heteroaryl and n is 0 then the monocyclic group that is B is substituted on one of the atoms adjacent to the atom to which the oxygen is attached, by a group selected from those listed above in the definition of B which optionally substitute B;
R¹ and R³ together with the nitrogen and carbon atoms to which they are respectively attached form a saturated 4- to 6-membered ring optionally containing a further heteroatom group selected from, NH, O, S or SO₂;
R⁴, R⁵, R⁶, R⁸, R⁹, R¹⁰, R¹² and R¹³ are independently hydrogen or methyl;
R⁷ is hydrogen or a group selected from C₁₋₆alkyl, C₃₋₇cycloalkyl, aryl, heteroaryl or heterocyclyl where the group is optionally substituted by heterocyclyl, aryl and heteroaryl; and wherein the group from which R⁷ may be selected is optionally substituted on the group and/or on its optional substituent by one or more substitutents independently selected from halo, cyano, C₁₋₄alkyl, —COC₁₋₄alkyl, —OR²¹, —NR²¹R²², —C₂R²¹, —NR²¹COR²², —NR²¹CO₂R²² and —CONR²¹R²²; and
R²¹ is hydrogen, methyl, ethyl, phenyl or benzyl;
R²² is hydrogen, methyl, ethyl, tert-butyl, phenyl or benzyl.

Another preferred class of compound is of the formula (IB) wherein:
Y¹ and Y² are both O;
z is NR⁸;
n is 0 or 1;
W is NR¹;
V is SO₂;
t is 1;
B is phenyl, naphthyl, pyridyl, imidazolyl, quinolinyl, cinnolyl, isoquinolinyl, thienopyridyl, naphthyridinyl, 2,5-methylenedioxyphenyl, 3,4-methylenedioxyphenyl, thienopyrimidinyl, pyrimidinyl, thienyl, pyrrolyl, pyrazolyl, thiazolyl, oxazolyl, isoxazolyl, pyrazinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinolinyl, tetrahydroisoquinolinyl and isoindolinyl, where each is optionally substituted by one or more groups independently selected from nitro, trifluoromethyl, trifluoromethoxy, halo, C₁₋₄alkyl (optionally substituted by one or more fluoro), C₂₋₄alkynyl, heteroaryl, —OR⁹, cyano, —NR⁹R¹⁰, —CONR⁹R¹⁰ and —NR⁹COR¹⁰; or B is vinyl or ethynyl optionally substituted by C₁₋₄alkyl;
R¹ and R³ are together with the itrogen and carbon atoms to which they are respectively attached form a saturated 5- to 6-membered ring optionally substituted on carbon by C₁₋₄alkyl, fluoro or C₁₋₄alkoxy;
R⁴, R⁵, R⁶, R⁸, R⁹, R¹⁰, R¹² and R¹³ are independently hydrogen or methyl;
R⁷ is hydrogen or a group selected from C₁₋₄alkyl, tetrahydrofuranyl, tetrahydropyranyl, pyrrolidinyl, piperidinyl, morpholinyl optionally substituted by one or more C₁₋₄alkoxy, fluoro, —COC₁₋₃alkyl or —SO₂C₁₋₃alkyl.

Another preferred class of compound is of the formula (IB) wherein:
Y¹ and Y² are both O;
z is NR⁸;
n is 0 or 1;
W is NR¹;
V is SO₂;
t is 1;
B is a group selected from quinolinyl, pyridyl and phenyl where each group is optionally substituted by 1 or 2 methyl, trifluoromethyl, trifluoromethoxy or halo;
R¹ and R³ together with the nitrogen and carbon atoms to which they are respectively attached form a saturated 5- to 6-membered ring;
R⁴, R⁵, R⁶, R¹² and R¹³ are independently hydrogen or methyl;
R⁷ is hydrogen, methyl or ethyl;
R²¹ is hydrogen, methyl, ethyl, phenyl and benzyl;
R²² is hydrogen, methyl, ethyl, tert-butyl, phenyl and benzyl.

In another aspect of the invention, preferred compounds of the invention are any one of:
1-(4-methyl-2,5-dioxoimidazolidin-4-yl)-N-{4-[(2-methylquinolin-4-yl)methoxy]phenyl}methanesulphonamide
1-(4-ethyl-2,5-dioxoimidazolidin-4-yl)-N-{4-[(2-methylquinolin-4-yl)methoxy]phenyl}methanesulphonamide
2-(2,5-dioxoimidazolidin-4-yl)-N-{4-[(2-methylquinolin-4-yl)methoxy]phenyl}ethanesulphonamide
N-{4-[(2,5-dimethylbenzyl)oxy]phenyl}-1-(4-methyl-2,5-dioxoimidazolidin-4-yl)methanesulphonamide
N-{4-[(2,5-dimethylbenzyl)oxy]phenyl}-1-(4-ethyl-2,5-dioxoimidazolidin-4-yl)methanesulphonamide
N-methyl-1-(4-methyl-2,5-dioxoimidazolidin-4-yl)-N-{4-[(2-methylquinolin-4-yl)methoxy]phenyl}methanesulphonamide (trifluoroacetic acid salt);
N-{4-[(2-methylquinolin-4-yl)methoxy]phenyl}-2,4-dioxo-1,3-diazaspiro[4.5]decane-6-sulphonamide;
N-{4-[(2-methylquinolin-4-yl)methoxy]phenyl}-2,4-dioxo-1,3-diazaspiro[4.4]nonane-6-sulphonamide;
N-{4-[(2-methylquinolin-4-yl)methoxy]phenyl}-2,4-dioxo-7-oxa-1,3-diazaspiro[4.4]nonane-9-sulphonamide;
5-[1-({4-[(2-methylquinolin-4-yl)methoxy]phenyl}sulphonyl)pyrrolidin-2-yl]imidazolidine-2,4-dione;
5-(1-{4[(2-methylquinolin-4-yl)methoxy]benzoyl}pyrrolidin-2-yl)imidazolidine-2,4-dione;
5-[1-({4-[(2-methylquinolin-4-yl)methoxy]phenyl}sulphonyl)piperidin-2-yl]imidazolidine-2,4-dione; and
(5R)-5-methyl-5-[(2R)-1-({4-[(2-methylquinolin-4-yl)methoxy]phenyl}sulphonyl)pyrrolidin-2-yl]imidazoline-2,4-dione.

Preferred compounds of formula (IA) are:
1-(4-methyl-2,5-dioxoimidazolidin-4-yl)-N-{4-[(2-methylquinolin-4-yl)methoxy]phenyl}methanesulphonamide
1-(4-ethyl-2,5-dioxoimidazolidin-4-yl)-N-{4-[(2-methylquinolin-4-yl)methoxy]phenyl}methanesulphonamide
2-(2,5-dioxoimidazolidin-4-yl)-N-{4-[(2-methylquinolin-4-yl)methoxy]phenyl}ethanesulphonamide
N-{4-[(2,5-dimethylbenzyl)oxy]phenyl}-1-(4-methyl-2,5-dioxoimidazolidin-4-yl)methanesulphonamide
N-{4-[(2,5-dimethylbenzyl)oxy]phenyl}-1-(4-ethyl-2,5-dioxoimidazolidin-4-yl)methanesulphonamide
N-methyl-1-(4-methyl-2,5-dioxoimidazolidin-4-yl)-N-{4-[(2-methylquinolin-4-yl)methoxy]phenyl}methanesulphonamide (trifluoroacetic acid salt);
N-{4-[(2-methylquinolin-4-yl)methoxy]phenyl}-2,4-dioxo-1,3-diazaspiro[4.5]decane-6-sulphonamide;
N-{4-[(2-methylquinolin-4-yl)methoxy]phenyl}-2,4-dioxo-1,3-diazaspiro[4.4]nonane-6-sulphonamide; and
N-{4-[(2-methylquinolin-4-yl)methoxy]phenyl}-2,4-dioxo-7-oxa-1,3-diazaspiro[4.4]nonane-9-sulphonamide.

Preferred compounds of formula (IB) are:
5-[1-({4-[(2-methylquinolin-4-yl)methoxy]phenyl}sulphonyl)pyrrolidin-2-yl]imidazolidine-2,4-dione;
5-[1-({4-[(2-methylquinolin-4-yl)methoxy]phenyl}sulphonyl)piperidin-2-yl]imidazolidine-2,4-dione; and
(5R)-5-methyl-5-[(2R)-1-({4-[(2-methylquinolin-4-yl)methoxy]phenyl}sulphonyl)pyrrolidin-2-yl]imidazoline-2,4-dione.

In another aspect the present invention provides a process for the preparation of a compound of formula (IA) or (IB) or a pharmaceutically acceptable salt or in vivo hydrolysable ester thereof wherein Y¹ and Y² are both O, z is NR⁸ and R⁸ is hydrogen, which comprises converting a ketone or aldehyde of formula (IIA) or (IIB) into a compound of formula (IA) or (IB); 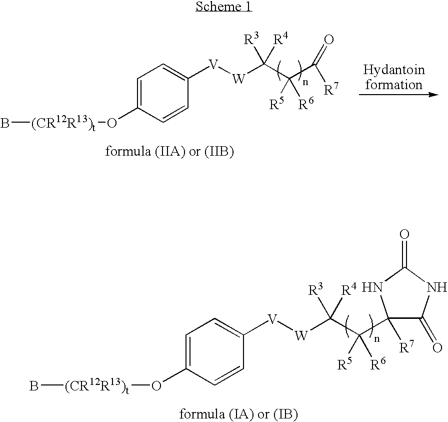
and thereafter if necessary:
i) converting a compound of formula (IA) or (IB) into another compound of the formula (IA) or (IB);
ii) removing any protecting groups;
iii) forming a pharmaceutically acceptable salt or in vivo hydrolysable ester.
   The hydantoin can be prepared by a number of methods, for example;
a) The aldehyde or ketone may be reacted with ammonium carbonate and potassium cyanide in aqueous alcohols using the method of Bucherer and Bergs (Adv. Het. Chem., 1985, 38, 177).
b) The aldehyde or ketone could be first converted to the cyanohydrin and then further reacted with ammonium carbonate (Chem. Rev, 1950, 56, 403).
c) The aldehyde or ketone could be converted to the alpha-amino nitrile and then either reacted with ammonium carbonate or aqueous carbon dioxide or potassium cyanate followed by mineral acid (Chem. Rev, 1950, 56,403).

A ketone or aldehyde of formula (IIB) may be prepared by a process comprising converting a compound of formula (IIIB) (where R is C₁₋₁₀alkyl and X is O or XR is NHOMe) into an aldehyde or ketone of formula (IIB); 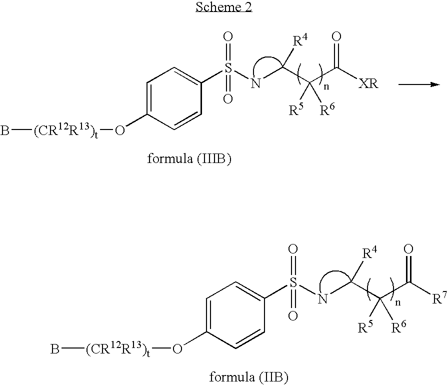
Suitable reagents for such a transformation are Grignard reagents of formula R⁷MgX (where X is halo) to prepare ketones or diisobutylaluminium hydride in dichloromethane at −78° C. under an argon atmosphere to prepare aldehydes.

A compound of formula (IIIB) can be prepared by reaction of a compound of formula (IVB) with a compound of formula (VB) or its salt under standard sulphonamide formation conditions (e.g. triethylamine in dichloromethane at temperatures from 0° C. to 50° C.); 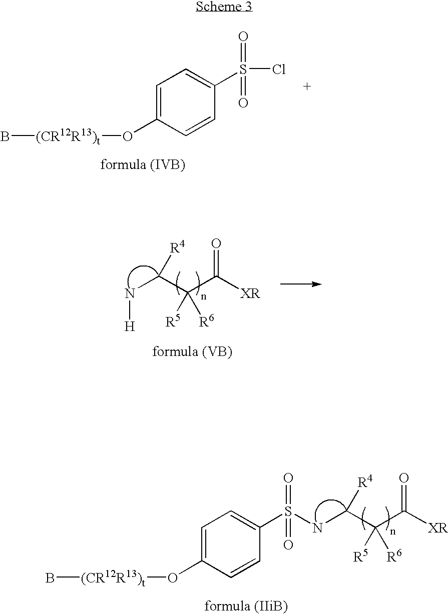
Many compounds of formula (VB) are commercially available or can be easily prepared by the skilled person. The sulphonyl chloride of formula (IVB) can be prepared as outlined in Scheme 4 which comprises; 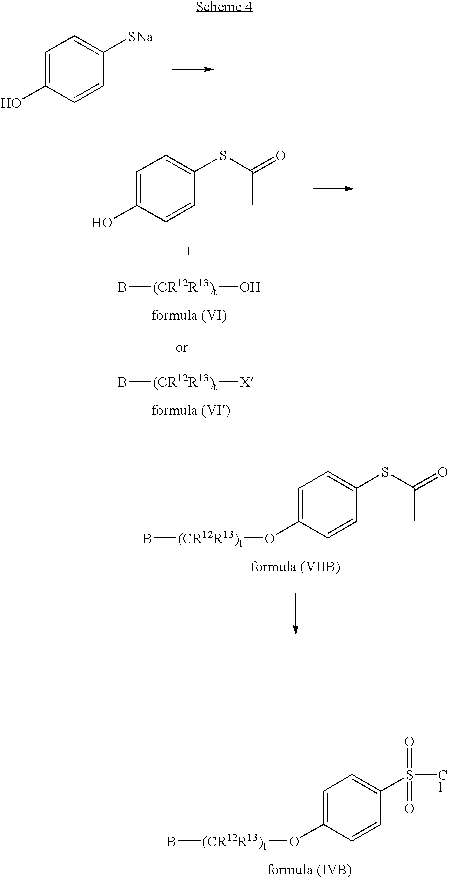
a) reacting the monosodium salt of 4-mercaptophenol with acetic anhydride (J. Am. Chem. Soc., 1956, 78, 854.) to yield S-(4-hydroxyphenyl)ethanethioate;
b) reacting S-(4-hydroxyphenyl)ethanethioate with an alcohol of formula (VI) under Mitsunobu type conditions or with a halide of formula (VI′) (where X′ is halo) by deprotonation with a base such as sodium hydride, lithium bis(trimethylsilyl)amide or caesium carbonate in a solvent such as dichloromethane, dimethylformamide, tetrahydrofuran or dimethyl sulphoxide at 0° C. to 100° C. to give a compound of formula (VIIB); and
c) oxidising a compound of formula (VIIB) by bubbling chlorine gas into a solution of the thiol ester in glacial acetic acid at temperatures from 0° C. to room temperature to yield the sulphonyl chloride of formula (IVB).

In another aspect the present invention provides a process for the preparation of a compound of formula (IB) or a pharmaceutically acceptable salt or in vivo hydrolysable ester, which process comprises coupling a sulphonyl chloride of formula (IVB) with an amine of formula (VIIIB) under standard sulphonamide formation conditions and followed by deprotection. 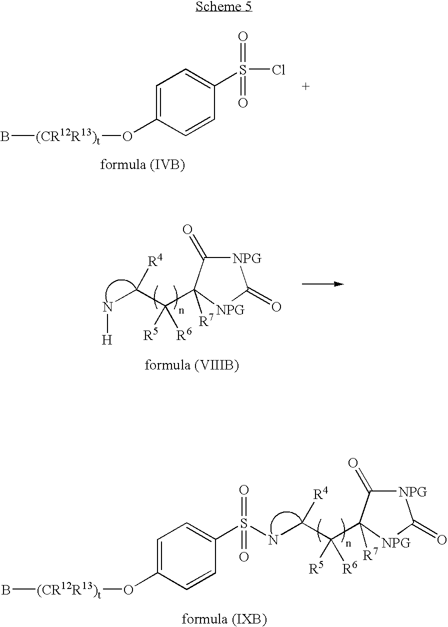
Also provided is a process for the preparation of an amine of formula (VIIIB) as shown in Scheme 6 which comprises the steps of: 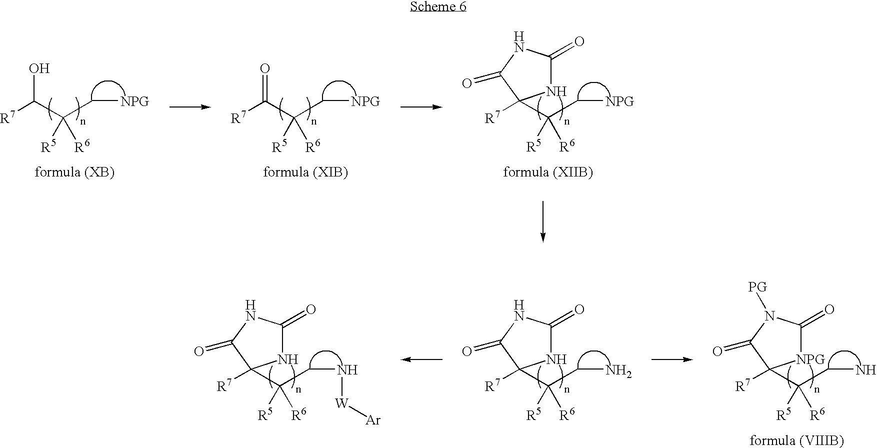
a) reacting a protected amino alcohol of formula (XB) with an oxidising agent to give a protected amino ketone or aldehyde of formula (XIB);
b) reacting the ketone or aldehyde under hydantion formation conditions to give a protected amine of formula (XIIB); and
c) removing and adding protecting groups as required to yield an amine of formula (VIIIB).
   A sulphonamide may be obtained by reacting the hydantoin with a sulphonyl chloride, acyl chloride or activated ester.
   The amine of formula (VIIIB) may also be obtained by a the process shown in scheme 6a which comprises the steps of: 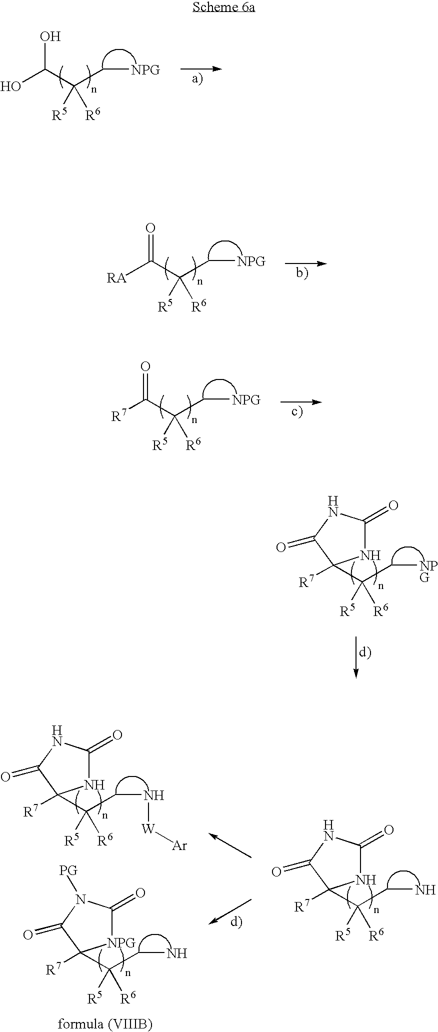
a) reacting a protected amino acid with either an alcohol under non aqueous acidic conditions, or under basic conditions with an alkylating agent to provide the ester (where A is O), or reacting the acid with N,O-dimethlyhydroxylamine hydrochloride under standard amide coupling conditions, or by reacting with triphenylphosphine, carbon tetrabromide and triethylamine in dichlormethane for 10 to 60 min (Synth. Commun., 1990, 20, 1105), to give an amide (where A is NH);
b) reacting the ester or amide of step a) with a Grignard (R⁷MgX) or alkyllithium (R⁷Li) reagent, or reducing agents to provide either the ketone or the aldehyde;
c) reacting the ketone or aldehyde from step b) under hydantion formation conditions to give a hydantoin;
d) removing and adding protecting groups as necessary to yield an amine of formula (VIIIB);
   A sulphonamide may be obtained by reacting the hydantoin with a sulphonyl chloride, acyl chloride or activated ester.

There is also provided a process for the preparation of a compound of formula (IA), which process comprises: 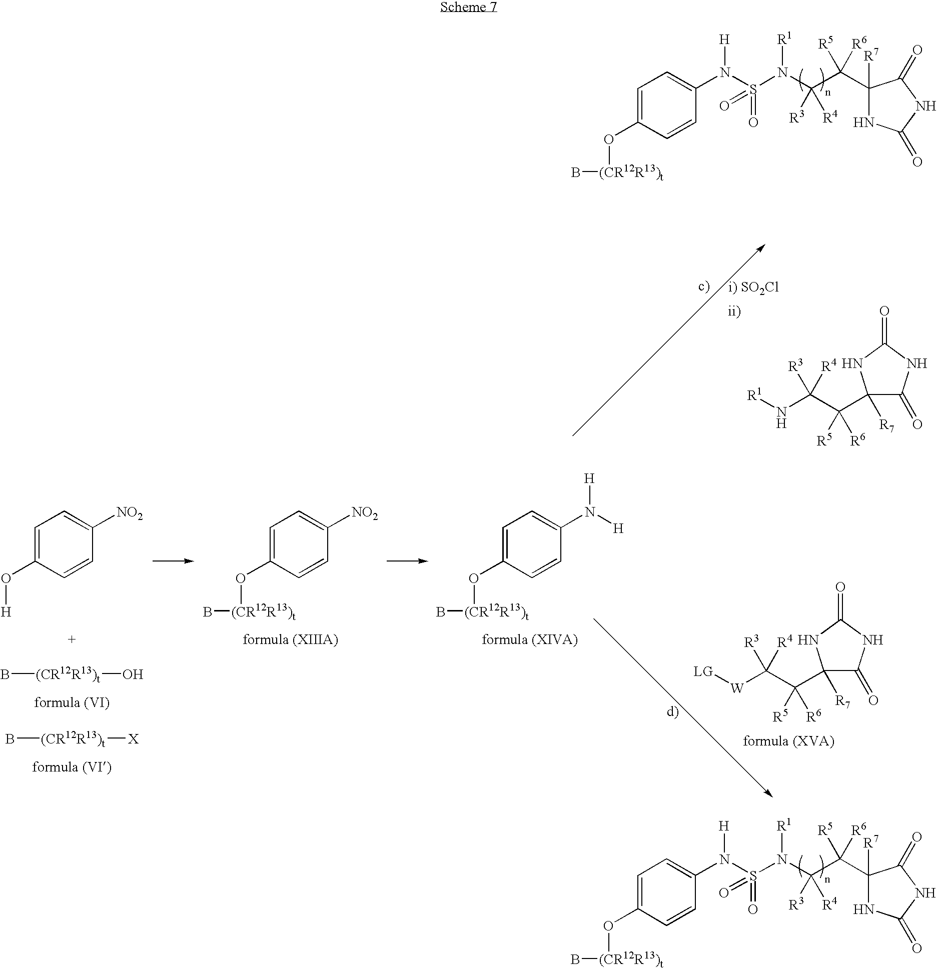
a) alkylating 4-nitrophenol with a compound of formula (VI′) where X is a leaving group (e.g. halo (Cl or Br) or mesyl) by deprotoning with a base such as sodium hydride, lithium bis(trimethylsilyl)amide or caesium carbonate in a solvent such as dichloromethane, tetrahydrofuran or dimethylsulphoxide at 0° C. to 100° C. or with a Mitsunobu reaction with a compound of formula (VI) to yield a compound of formula (XIIIA);
b) reducing the nitro group of the compound of formula (XIIIA) using e.g. Zn/HCl or SnCl₂/HCl to yield a compound of formula (XIVA); then
c) forming the sulphonamide (when W is NR¹) by reacting the compound of formula (XIVA) with SO₂Cl₂ in dichloromethane at temperatures from −78° C. to room temperature to form a chlorosulphonamide intermediate followed by addition of an amine of formula (VIIIA) using standard sulphonamide formation conditions, e.g. in dichloromethane with triethylamine; or
d) forming the sulphonamide (when W is a bond or CR¹R²) by reacting the compound of formula (XIVA) with SO₂Cl₂ in dichloromethane at temperatures from −78° C. to room temperature to form a chlorosulphonamide intermediate followed by addition of a hydantoin sulphonyl chloride of formula (XVA) using standard sulphonamide formation conditions, e.g. in dichloromethane with triethylamine.

Further aspects of the invention include a process for preparing a compound of formula (IA) which when W is NR¹ comprises: 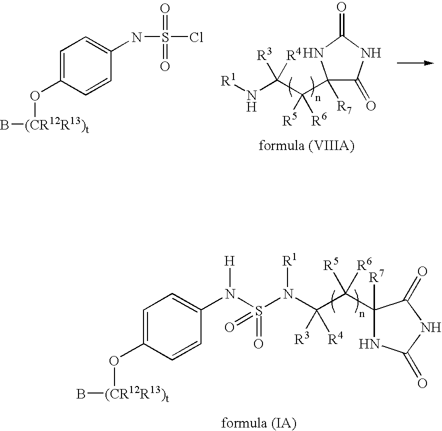
reaction of an amine of formula (VIIIA) with a suitable chlorosulphonamide intermediate under standard sulphonamide formation conditions (as described above in c)); or when W is a bond or CR¹R², comprises 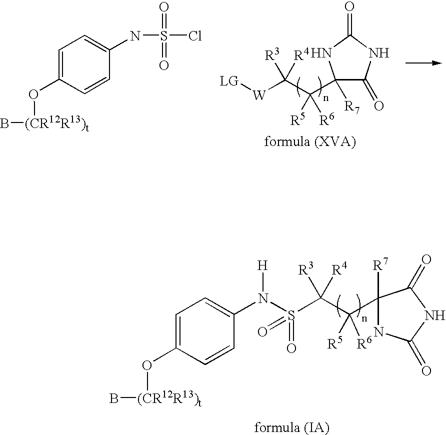
reaction of a hydantoin sulphonyl chloride of formula (XVA) with a suitable chlorosulphonamide intermediate under standard sulphonamide formation conditions (as described above in d));
and thereafter if necessary:
i) converting a compound of the formula (IA) into another compound of the formula (IA);
ii) removing any protecting groups;
iii) forming a pharmaceutically acceptable salt or in vivo hydrolysable ester.

An amine of formula (VIIIA) may be obtained by processes that are analogous to those shown in schemes 6 and 6a for the preparation of an amine of formula (VIIIB) or its deprotected analogue.

A sulphonyl chloride of formula (XVA) can be formed as follows: 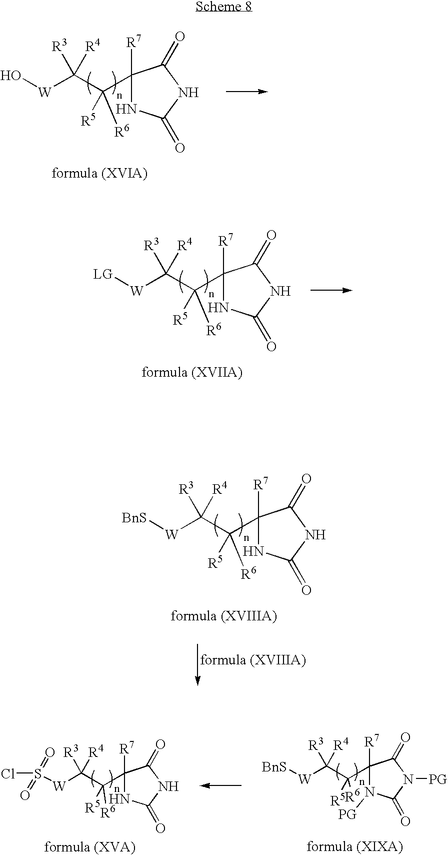
The process of Scheme 8 comprises the steps of:
a) transforming the hydroxy hydantoin of formula (XVIA) (which can be prepared by standard methods from aldehydes and ketones as described above) into a leaving group (LG) using, for example, tosyl chloride, mesyl chloride in dichloromethane with triethylamine to yield a compound of formula (XVIIA);
b) displacing the LG using the anion of benzylthiol (deprotonated using sodium hydride) in tetrahydrofuran to yield a compound of formula (XVIIIA);
c) protecting the hydantoin with a protecting group e.g. benzyl using benzyl bromide and sodium hydride in tetrahydrofuran; and
e) treating the benzylthioether of formula (XIXA) with chlorine gas in aqueous acetic acid to yield the sulphonyl chloride of formula (XVA).

A compound of formula (IA) or (IB) can be prepared by removal of protecting groups on the hydantoin directly. The protecting group can be tert-butyloxycarbonyl (BOC), benzyl (Bn) or benzyloxycarbonyl (cbz). These can be removed by treatment with trifluoroacetic acid or HCl in dioxane for the former or by treatment with palladium/hydrogen for the latter two.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogen group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possesses metalloproteinases inhibitory activity, and in particular TACE inhibitory activity. This property may be assessed, for example, using the procedure set out below.

Isolated Enzyme Assays

Matrix Metalloproteinase Family Including for Example MMP13.

Recombinant human proMMP13 may be expressed and purified as described by Knauper et al. [V. Knauper et al., (1996) The Biochemical Journal 271:1544-1550 (1996)]. The purified enzyme can be used to monitor inhibitors of activity as follows: purified proMMP13 is activated using 1 mM amino phenyl mercuric acid (APMA), 20 hours at 21° C.; the activated MMP13 (11.25 ng per assay) is incubated for 4-5 hours at 35° C. in assay buffer (0.1M Tris-HCl, pH 7.5 containing 0.1M NaCl, 20 mM CaCl₂, 0.02 mM ZnCl and 0.05% (w/v) Brij 35 using the synthetic substrate 7-methoxycoumarin-4-yl)acetyl.Pro.Leu.Gly.Leu.N-3-(2,4-dinitrophenyl)-L-2,3-diaminopropionyl.Ala.Arg.NH₂ in the presence or absence of inhibitors. Activity is determined by measuring the fluorescence at λex 328 nm and λem 393 nm. Percent inhibition is calculated as follows: % Inhibition is equal to the [Fluorescence_{plus inhibitor}−Fluorescence_{background}] divided by the [Fluorescence_{minus inhibitor}−Fluorescence_{background}].

A similar protocol can be used for other expressed and purified pro MMPs using substrates and buffers conditions optimal for the particular MMP, for instance as described in C. Graham Knight et al., (1992) FEBS Lett. 296(3):263-266.

Adamalysin Family Including for Example TNF Convertase

The ability of the compounds to inhibit proTNF-α convertase enzyme (TACE) may be assessed using a partially purified, isolated enzyme assay, the enzyme being obtained from the membranes of THP-1 as described by K. M. Mohler et al., (1994) Nature 370:218-220. The purified enzyme activity and inhibition thereof is determined by incubating the partially purified enzyme in the presence or absence of test compounds using the substrate 4′,5′-Dimethoxy-fluoresceinyl Ser.Pro.Leu.Ala.Gln.Ala.Val.Arg.Ser.Ser.Ser.Arg.Cys(4-(3-succinimid-1-yl)-fluorescein)-NH₂ in assay buffer (50 mM Tris HCl, pH 7.4 containing 0.1% (w/v) Triton X-100 and 2 mM CaCl₂), at 26° C. for 4 hours. The amount of inhibition is determined as for MMP13 except λex 485 nm and λem 538 nm were used. The substrate was synthesised as follows. The peptidic part of the substrate was assembled on Fmoc-NH-Rink-MBHA-polystyrene resin either manually or on an automated peptide synthesiser by standard methods involving the use of Fmoc-amino acids and O-benzotriazol-1-yl-N,N,N′,N′-tetramethyluronium hexafluorophosphate (HBTU) as coupling agent with at least a 4- or 5-fold excess of Fmoc-amino acid and HBTU. Ser¹ and Pro² were double-coupled. The following side chain protection strategy was employed; Ser¹(But), Gln⁵(Trityl), Arg^{8,12}(Pmc or Pbf), Ser^{9,10,11}(Trityl), Cys¹³(Trityl). Following assembly, the N-terminal Fmoc-protecting group was removed by treating the Fmoc-peptidyl-resin with in DMF. The amino-peptidyl-resin so obtained was acylated by treatment for 1.5-2 hr at 70° C. with 1.5-2 equivalents of 4′,5′-dimethoxy-fluorescein-4(5)-carboxylic acid [Khanna & Ullman, (1980) Anal Biochem. 108:156-161) which had been preactivated with diisopropylcarbodiimide and 1-hydroxybenzotriazole in DMF]. The dimethoxyfluoresceinyl-peptide was then simultaneously deprotected and cleaved from the resin by treatment with trifluoroacetic acid containing 5% each of water and triethylsilane. The dimethoxyfluoresceinyl-peptide was isolated by evaporation, trituration with diethyl ether and filtration. The isolated peptide was reacted with 4-(N-maleimido)-fluorescein in DMF containing diisopropylethylamine, the product purified by RP-HPLC and finally isolated by freeze-drying from aqueous acetic acid. The product was characterised by MALDI-TOF MS and amino acid analysis.

The compounds of this invention are active against TACE (causing at least 50% inhibition) at less than 10 μM. In particular compound 1A gave 50% inhibition at 71 nM and compound 2A gave 50% inhibition at 37 nM.

Natural Substrates

The activity of the compounds of the invention as inhibitors of aggrecan degradation may be assayed using methods for example based on the disclosures of E. C. Arner et al., (1998) Osteoarthritis and Cartilage 6:214-228; (1999) Journal of Biological Chemistry, 274 (10), 6594-6601 and the antibodies described therein. The potency of compounds to act as inhibitors against collagenases can be determined as described by T. Cawston and A. Barrett (1979) Anal. Biochem. 99:340-345.

Inhibition of Metalloproteinase Activity in Cell/Tissue Based Activity

Test as an Agent to Inhibit Membrane Sheddases such as TNF Convertase

The ability of the compounds of this invention to inhibit the cellular processing of TNP-α production may be assessed in THP-1 cells using an ELISA to detect released TNF essentially as described K. M. Mohler et al., (1994) Nature 370:218-220. In a similar fashion the processing or shedding of other membrane molecules such as those described in N. M. Hooper et al., (1997) Biochem. J. 321:265-279 may be tested using appropriate cell lines and with suitable antibodies to detect the shed protein.

Test as an Agent to Inhibit Cell Based Invasion

The ability of the compound of this invention to inhibit the migration of cells in an invasion assay may be determined as described in A. Albini et al., (1987) Cancer Research 47:3239-3245.

Test as an Agent to Inhibit Whole Blood TNF Sheddase Activity

The ability of the compounds of this invention to inhibit TNF-α production is assessed in a human whole blood assay where LPS is used to stimulate the release of TNF-α. 160 μl of heparinized (10 Units/ml) human blood obtained from volunteers, was added to the plate and incubated with 20 μl of test compound (duplicates), in RPMI1640+bicarbonate, penicillin, streptomycin, glutamine and 1% DMSO, for 30 min at 37° C. in a humidified (5% CO₂/95% air) incubator, prior to addition of 20 μL LPS (E. coli. 0111:B4; final concentration 10 μg/ml). Each assay includes controls of neat blood incubated with medium alone or LPS (6 wells/plate of each). The plates are then incubated for 6 hours at 37° C. (humidified incubator), centrifuged (2000 rpm for 10 min; 4° C.), plasma harvested (50-100 μl) and stored in 96 well plates at −70° C. before subsequent analysis for TNF-α concentration by ELISA.

Test as an Agent to Inhibit In Vitro Cartilage Degradation

The ability of the compounds of this invention to inhibit the degradation of the aggrecan or collagen components of cartilage can be assessed essentially as described by K. M. Bottomley et al., (1997) Biochem J. 323:483-488.

In Vivo Assessment

Test as an Anti-TNF Agent

The ability of the compounds of this invention as in vivo TNF-α inhibitors is assessed in the rat. Briefly, groups of female Wistar Alderley Park (AP) rats (90-100 g) are dosed with compound (5 rats) or drug vehicle (5 rats) by the appropriate route e.g. peroral (p.o.), intraperitoneal (i.p.), subcutaneous (s.c.) 1 hour prior to lipopolysaccharide (LPS) challenge (30 μg/rat i.v.). Sixty minutes following LPS challenge rats are anaesthetised and a terminal blood sample taken via the posterior vena cavae. Blood is allowed to clot at room temperature for 2 hours and serum samples obtained. These are stored at −20° C. for TNF-α ELISA and compound concentration analysis.

Data analysis by dedicated software calculates for each compound/dose:

$$Percent inhibition of TNF - α = \frac{Mean TNF - α ( Vehicle control ) - Mean TNF - α ( Treated ) \times 100}{Mean TNF - α ( Vehicle control )}$$

Test as an Anti-Arthritic Agent

Activity of a compound as an anti-arthritic is tested in the collagen-induced arthritis (CIA) as defined by D. E. Trentham et al., (1977) J. Exp. Med. 146:857. In this model acid soluble native type II collagen causes polyarthritis in rats when administered in Freunds incomplete adjuvant. Similar conditions can be used to induce arthritis in mice and primates.

Pharmaceutical Compositions

According to a further aspect of the invention there is provided a pharmaceutical composition which comprises a compound of the formula (I), (IA) or (IB), or a pharmaceutically acceptable salt or in vivo hydrolysable ester thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository. The composition may also be in a form suitable for inhalation.

In general the above compositions may be prepared in a conventional manner using conventional excipients.

The pharmaceutical compositions of this invention will normally be administered to humans so that, for example, a daily dose of 0.5 to 75 mg/kg body weight (and preferably 0.5 to 30 mg/kg body weight) is received. This daily dose may be given in divided doses as necessary, the precise amount of the compound received and the route of administration depending on the weight, age and sex of the patient being treated and on the particular disease condition being treated according to principles known in the art.

Typically unit dosage forms will contain about 1 mg to 500 mg of a compound of this invention.

Therefore in a further aspect of the present invention there is provided a compound of the formula (I), (IA) or (IB), or a pharmaceutically acceptable salt or in vivo hydrolysable ester thereof, as defined hereinbefore, for use in a method of treatment of a warm-blooded animal such as man by therapy.

Also provided is a compound of the formula (I), (IA) or (IB), or a pharmaceutically acceptable salt or in vivo hydrolysable ester thereof, as defined hereinbefore, for use in a method of treating a disease condition mediated by one or more metalloproteinase enzymes and in particular a disease condition mediated by TNF.

Further provided is a compound of the formula (I), (IA) or (IB), or a pharmaceutically acceptable salt or in vivo hydrolysable ester thereof, as defined hereinbefore, for use in a method of treating inflammatory diseases, autoimmune diseases, allergic/atopic diseases, transplant rejection, graft versus host disease, cardiovascular disease, reperfusion injury and malignancy in a warm-blooded animal such as man. In particular a compound of the formula (I), (IA) or (IB), or a pharmaceutically acceptable salt or in vivo hydrolysable ester thereof, as defined hereinbefore, is provided for use in a method of treating rheumatoid arthritis, Crohn's disease and psoriasis, and especially rheumatoid arthritis in a warm-blooded animal such as man. Also provided is a compound of formula (I), (IA) or (IB), or a pharmaceutically acceptable salt or in vivo hydrolysable ester thereof, as defined hereinbefore, for use in a method of treating a respiratory disorder such as asthma or COPD in a warm-blooded animal such as man.

According to an additional aspect of the invention there is provided a compound of formula (I), (IA) or (IB) or a pharmaceutically acceptable salt or in vivo hydrolysable ester thereof, as defined hereinbefore, for use as a medicament. Also provided is a compound of the formula (I), (IA) or (IB) or a pharmaceutically acceptable salt or in vivo hydrolysable ester thereof, as defined hereinbefore, for use as a medicament in the treatment of a disease condition mediated by one or more metalloproteinase enzymes and in particular a disease condition mediated by TNF-α. Further provided is a compound of the formula (I), (IA) or (IB), or a pharmaceutically acceptable salt or in vivo hydrolysable ester thereof, as defined hereinbefore, for use as a medicament in the treatment of inflammatory diseases, autoimmune diseases, allergic/atopic diseases, transplant rejection, graft versus host disease, cardiovascular disease, reperfusion injury and malignancy in a warm-blooded animal such as man. In particular a compound of the formula (I1), or a pharmaceutically acceptable salt or in vivo hydrolysable ester thereof, as defined hereinbefore, is provided for use as a medicament in the treatment of rheumatoid arthritis, Crohn's disease and psoriasis, and especially rheumatoid arthritis in a warm-blooded animal such as man. Also provided is a compound of the formula (I), (IA) or (IB), or a pharmaceutically acceptable salt or in vivo hydrolysable ester thereof, as defined hereinbefore, for use as a medicament in the treatment a respiratory disorder such as asthma or COPD in a warm-blooded animal such as man.

According to this another aspect of the invention there is provided the use of a compound of the formula (I), (IA) or (IB), or a pharmaceutically acceptable salt or in vivo hydrolysable ester thereof, as defined hereinbefore in the manufacture of a medicament for use in the treatment of a disease condition mediated by one or more metalloproteinase enzymes and in particular a disease condition mediated by TNF-α in a warm-blooded animal such as man. Also provided is the use of a compound of the formula (I), (IA) or (IB), or a pharmaceutically acceptable salt or in vivo hydrolysable ester thereof, as defined hereinbefore in the manufacture of a medicament for use in the treatment of inflammatory diseases, autoimmune diseases, allergic/atopic diseases, transplant rejection, graft versus host disease, cardiovascular disease, reperfusion injury and malignancy in a warm-blooded animal such as man. In particular the use of a compound of the formula (I), (IA) or (IB), or a pharmaceutically acceptable salt or in vivo hydrolysable ester thereof, as defined hereinbefore, is provided in the manufacture of a medicament in the treatment of rheumatoid arthritis, Crohn's disease and psoriasis, and especially rheumatoid arthritis in a warm-blooded animal such as man. Further provided is the use of a compound of the formula (I), (IA) or (IB), or a pharmaceutically acceptable salt or in vivo hydrolysable ester thereof, as defined hereinbefore in the manufacture of a medicament for use in the treatment a respiratory disorder such as asthma or COPD in a warm-blooded animal such as man.

According to a further feature of this aspect of the invention there is provided a method of producing a metalloprotienase inhibitory effect in a warm-blooded animal, such as man, in need of such treatment which comprises administering to said animal an effective amount of a compound of formula (I), (IA) or (IB). According to a further feature of this aspect of the invention there is provided a method of producing a TACE inhibitory effect in a warm-blooded animal, such as man, in need of such treatment which comprises administering to said animal an effective amount of a compound of formula (I), (A) or (IB). According to this further feature of this aspect of the invention there is provided a method of treating autoimmune disease, allergic/atopic diseases, transplant rejection, graft versus host disease, cardiovascular disease, reperfusion injury and malignancy in a warm-blooded animal, such as man, in need of such treatment which comprises administering to said animal an effective amount of a compound of formula (I), (IA) or (IB). Also provided is a method of treating rheumatoid arthritis, Crohn's disease and psoriasis, and especially rheumatoid arthritis in a warm-blooded animal, such as man, in need of such treatment which comprises administering to said animal an effective amount of a compound of formula (I), (IA) or (IB). Further provided is a method of treating a respiratory disorder such as asthma or COPD in a warm-blooded animal, such as man, in need of such treatment which comprises administering to said animal an effective amount of a compound of formula (I), (IA) or (IB).

In addition to their use in therapeutic medicine, the compounds of formula (I), (IA) or (IB) and their pharmaceutically acceptable salts are also useful as pharmacological tools in the development and standardisation of in vitro and in vivo test systems for the evaluation of the effects of inhibitors of cell cycle activity in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

The compounds of this invention may be used in combination with other drugs and therapies used in the treatment of various immunological, inflammatory or malignant disease states which would benefit from the inhibition of TACE.

If formulated as a fixed dose such combination products employ the compounds of this invention within the dosage range described herein and the other pharmaceutically-active agent within its approved dosage range. Sequential use is contemplated when a combination formulation is inappropriate.

### EXAMPLES

The invention will now be illustrated by the following non-limiting examples in which, unless stated otherwise:
(i) temperatures are given in degrees Celsius (° C.); operations were carried out at room or ambient temperature, that is, at a temperature in the range of 18-25° C.;
(ii) organic solutions were dried over anhydrous magnesium sulphate; evaporation of solvent was carried out using a rotary evaporator under reduced pressure (600-4000 Pascals; 4.5-30 mm Hg) with a bath temperature of up to 60° C.;
(iii) chromatography unless otherwise stated means flash chromatography on silica gel; thin layer chromatography (TLC) was carried out on silica gel plates; where a “Bond Elut” column is referred to, this means a column containing 10 g or 20 g of silica of 40 micron particle size, the silica being contained in a 60 ml disposable syringe and supported by a porous disc, obtained from Varian, Harbor City, Calif., USA under the name “Mega Bond Elut SI”. Where an “Isolute™ SCX column” is referred to, this means a column containing benzenesulphonic acid (non-endcapped) obtained from International Sorbent Technology Ltd., 1st House, Duffryn Industial Estate, Ystrad Mynach, Hengoed, Mid Glamorgan, UK. Where Flashmaster II is referred to, this means a UV driven automated chromatography unit supplied by Jones;
(iv) in general, the course of reactions was followed by TLC and reaction times are given for illustration only;
(v) yields, when given, are for illustration only and are not necessarily those which can be obtained by diligent process development; preparations were repeated if more material was required;
(vi) when given, ¹H NMR data is quoted and is in the form of delta values for major diagnostic protons, given in parts per million (ppm) relative to tetramethylsilane (TMS) as an internal standard, determined at 400 MHz using CDCl₃ as the solvent unless otherwise stated; coupling constants (J) are given in Hz;
(vii) chemical symbols have their usual meanings; SI units and symbols are used;
(viii) solvent ratios are given in percentage by volume;
(ix) mass spectra (MS) were run with an electron energy of 70 electron volts in the chemical ionisation (APCI) mode using a direct exposure probe; where indicated ionisation was effected by electrospray (ES); where values for m/z are given, generally only ions which indicate the parent mass are reported, and unless otherwise stated the mass ion quoted is the positive mass ion—(M+H)⁺;
(x) LCMS (liquid chromatography mass spectrometry) characterisation was performed using a pair of Gilson 306 pumps with Gilson 233 XL sampler and Waters ZMD4000 mass spectrometer. The LC comprised water symmetry 4.6×50 column C18 with 5 micron particle size. The eluents were: A, water with 0.05% formic acid and B, acetonitrile with 0.05% formic acid. The eluent gradient went from 95% A to 95% B in 6 minutes. Where indicated ionisation was effected by electrospray (ES); where values for m/z are given, generally only ions which indicate the parent mass are reported, and unless otherwise stated the mass ion quoted is the positive mass ion—(M+H)⁺ and
(xi) the following abbreviations are used:
   DMSO dimethyl sulphoxide;
   DMF N,N-dimethylformamide;
   DCM dichloromethane;
   NMP N-methylpyrrolidin-2-one;
   DIAD diisopropyl azodicarboxylate
   LHMDS or LiHMDS lithium bis(trimethylsilyl)amide
   MeOH methanol
   RT room temperature
   TFA trifluoroacetic acid
   EtOH ethanol
   EtOAc ethyl acetate
   TBF tetrahydrofuran
   DIBAL diisobutylaluminium hydride
   AcOH acetic acid
   EDTA ethylenediaminetetraacetic acid
   PS-DMAP polymer supported 4-dimethylaminopyridine
   EDCI 1-(3-dimethylaminopropyl)-3-ethylcarbodiimide hydrochloride
      Compounds of Formula (IA)

### Example 1A

### 1-(4-methyl-2,5-dioxoimidazolidin-4-yl)-N-{4-[(2-methylquinolin-4-yl)methoxy]phenyl}methanesulphonamide

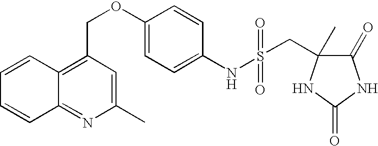

A mixture of [4-methyl-2,5-dioxoimidazolidin-4-yl]methanesulphonyl chloride (200 mg), 4-((2-methylquinolin-4-yl)methoxy)aniline (150 mg) and triethylamine (0.1 ml) in DMF (3 ml) was stirred at ambient temperature for 18 h. Additional [4-methyl-2,5-dioxoimidazolidin-4-yl]methanesulphonyl chloride (150 mg) and triethylamine (0.1 ml) were added and the mixture was stirred for 4 h before partitioning between water (50 ml) and EtOAc (100 ml). The organic phase was dried (MgSO₄), evaporated under vacuum and purified by column chromatography using DCM to 6% MeOH in DCM as the eluant. The product (127 mg) was triturated with diethylether to yield 1-(4-methyl-2,5-dioxoimidazolidin-4-yl)-N-{4-[(2-methylquinolin-4-yl)methoxy]phenyl}methanesulphonamide as a cream solid (71 mg); NMR DMSOd6 2.65 (3H, s), 1.29 (3H, s), 3.25 (1H, d), 3.45 (1H, d), 5.56 (2H, s), 7.07-7.18 (4H, m), 7.52-7.59 (2H, m), 7.72 (1H, t), 7.93-7.98 (2H, m), 8.09 (1H, d), 9.60 (1H, s), 10.69 (1H, bs); MS 455 (MH+).

The starting material [4-methyl-2,5-dioxoimidazolidin-4-yl]methanesulphonyl chloride was prepared as follows:
i) A steel vessel was charged with ethanol (315 ml) and water (135 ml). Benzylthioacetone (31.7 g, 0.175 mol), potassium cyanide (22.9 g, 0.351 mol) and ammonium carbonate (84.5 g, 0.879 mol) were added and reaction kept at 90° C. under vigorous stirring for 3 h. After cooling to 0° C. (0.5 h), the yellowish slurry was evaporated to dryness and the solid residue partitioned between water (400 ml) and EtOAc (700 ml) and separated. The water-phase was extracted with EtOAc (300 ml). The combined organic phases were washed with saturated brine (15 ml), dried (Na₂SO₄), filtered and evaporated to dryness. Crystallisation was assisted by the addition of DCM (300 ml) to the oil. Evaporation gave 5-methyl-5-{[(phenylmethyl)thio]methyl}imidazolidine-2,4-dione as a slightly yellowish powder (43.8 g, 90%); ¹H NMR (DMSOd6) 1.29 (3H, s), 3.76 (2H, s); 2.72, 2.62 (1H each, ABq, J=14.0 Hz); 7.35-7.20 (5H, m); 8.00 (1H, s); 10.74 (1H, s); MS 251.1 (MH+).
ii) 5-methyl-5-{[(phenylmethyl)thio]methyl}imidazolidine-2,4-dione (42.6 g; 0.17 mol) was dissolved in a mixture of AcOH (450 ml) and water (50 ml). The mixture was cooled to 0° C. and chlorine gas was bubbled through the solution such that the temperature was maintained at less than 15° C. After 25 min the solution became yellow-green in colour and a sample was withdrawn for LCMS and HPLC analysis. It showed that starting material had been consumed. The yellow clear solution was stirred for 30 min and an opaque solution/slurry was formed. The solvent was removed in vacuo at 37° C. and the resultant yellowish solid suspended in toluene (400 ml). Solvent was again removed. This was repeated once more. The crude product was then suspended in iso-hexane (400 ml) and warmed to 40° C. while stirring, after which the slurry was allowed to cool to RT before the insoluble product was removed by filtration, washed with iso-hexane (6×100 ml), and dried under in vacuo at 50° C. overnight. This gave [4-methyl-2,5-dioxoimidazolidin-4-yl]methanesulphonyl chloride as a slightly yellow powder (36.9 g, 95%);

Purity by HPLC=99%, NMR supported that purity; ¹H NMR (THF-d₈): δ 9.91 (1H, bs); 7.57 (1H, s); 4.53, 4.44 (1H each, ABq, J=14.6 Hz); 1.52 (s, 3H, CH₃); ¹³C NMR (THF-d₈): δ 174.96; 155.86; 70.96; 61.04; 23.66.

The starting material 4-[(2-methylquinolin-4-yl)methoxy]aniline was prepared as follows:
i) To a stirred suspension of 2-methylquinolin-4-ylcarboxylic acid (4 g, 21.4 mmol) in THF (100 ml) at RT was added lithium aluminium hydride (21.4 ml, 1.0M solution in THF, 21.4 mmol) dropwise over 20 min. After 16 h water (4 ml) was added cautiously followed by 2N NaOH (4 ml) and water (12 ml). The resulting gelatinous precipitate was filtered off and washed with THF. DCM (200 ml) was added to the filtrate and partitioned with saturated NaHCO₃ (2×75 ml). The organic layer was dried (MgSO₄), concentrated, triturated with DCM and filtered to give 2-methylquinolin-4-ylmethanol as a white powder (858 mg, 5 mmol). The mother liquours were purified by chromatography (20 g silica bond elute, eluent 0→5% EtOH/DCM) to give a further 610 mg of product (3.5 mmol); NMR: 2.6 (s, 3H), 5.0 (d, 2H), 5.5 (t, 1H), 7.4 (s, 1H), 7.5 (t, 1H), 7.7 (t, 1H) and 7.9 (m, 2H); MS: 174 (MH+).
ii) DIAD (24 ml) was added slowly to a mixture of 2-methylquinolin-4-ylmethanol (12 g), triphenylphosphine (31 g) and 4-nitrophenol (11.5 g) in THF (250 ml) keeping the temperature below 20° C. The mixture was stirred at ambient temperature for 18 h, diluted with DCM and applied to 170 g of SCX resin. This was washed with MeOH, 50% MeOH/50% DCM, DCM and 4% (7N ammonia in MeOH) in DCM. Fractions containing product were evaporated under vacuum to yield (2-methylquinolin-4-ylmethoxy)₄-nitrophenyl as a cream solid (19.5 g); NMR CDCl₃ 2.77 (3H, s), 5.61 (2H, s), 7.12 (2H, d), 7.42 (1H, s), 7.52-7.62 (1H, m), 7.71-7.79 (1H, m), 7.91 (1H, d), 8.10 (1H, d), 8.25 (2H, d); MS 295 (MH+).
iii) (2-Methylquinolin-4-ylmethoxy)-4-nitrophenyl (19.5 g) was reduced in batches by the following method. Nickel acetate (75 mg) was added to a suspension of borohydride resin (6.2 g) in MeOH (20 ml). The resin turned from gold to black and a solution/suspension of (2-methylquinolin-4-ylmethoxy)₄-nitrophenyl (900 mg) in MeOH (20 ml) at 60° C. was added to it. The mixture was stirred at 40° C. for 1 h before removing the resin by filtration. The combined filtrates were evaporated under reduced pressure to yield a gum, which was partitioned between DCM and aqueous EDTA solution (volumes and molarity not recorded). The organic phase was dried (Na₂SO₄), evaporated under reduced pressure and purified by column chromatography, using a gradient of isohexane to EtOAc to 3% MeOH in EtOAc as the eluant, to yield 4-[(2-methylquinolin-4-yl)methoxy]aniline as a yellow solid (11.65 g); NMR CDCl₃ 2.73 (3H, s), 3.49 (2H, bs), 5.42 (2H, s), 6.65 (2H, d), 6.86 (2H, d), 7.43-7.55 (2H, m), 7.65-7.73 (1H, m), 7.92 (1H, d), 8.05 (1H, d).

### Example 2A

### 1-(4-ethyl-2,5-dioxoimidazolidin-4-yl)-N-{4-[(2-methylquinolin-4-yl)methoxy]phenyl}methanesulphonamide

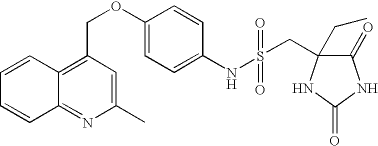

A mixture of [4-ethyl-2,5-dioxoimidazolidin-4-yl]methanesulphonyl chloride (211 mg), 4-((2-methylquinolin-4-yl)methoxy)aniline (example 1A, 150 mg) and triethylamine (0.1 ml) in DMF (3 ml) was stirred at ambient temperature for 18 h. Additional 4-[(2-methylquinolin-4-yl)methoxy]benzenesulphonyl chloride (150 mg) and triethylamine (0.1 ml) were added and the mixture was stirred for 4 h before partitioning between water (50 ml) and EtOAc (100 ml). The organic phase was dried (MgSO₄), evaporated under vacuum and purified by column chromatography using DCM to 6% MeOH in DCM as the eluant. The product (127 mg) was triturated with diethylether to yield 1-(4-ethyl-2,5-dioxoimidazolidin-4-yl)-N-{4-[(2-methylquinolin-4-yl)methoxy]phenyl}methanesulphonamide as a cream solid (71 mg); NMR DMSOd6 0.73 (3H, t), 1.52-1.66 (2H, m), 2.65 (3H, s), 3.23 (1H, d), 3.45 (1H, d), 5.55 (2H, s), 7.06-7.20 (4H, m), 7.51-7.60 (2H, m), 7.72 (1H, t), 7.90-7.98 (2H, m), 8.09 (1H, d), 9.58 (1H, bs), 10.707 (1H, bs); MS 469 (MH+).

The starting material [4-ethyl-2,5-dioxoimidazolidin-4-yl]methanesulphonyl chloride was prepared by an analogous method to that described in example 1A using steps i) and ii) for the preparation of [4-methyl-2,5-dioxoimidazolidin-4-yl]methylsulphonyl chloride except that 1-(benzylthio)butan-2-one (Tetrahedron Letters (1998), 39(20), 3189-3192) was used in place of benzylthioacetone; NMR (THFd8) 0.9 (3H, t), 1.9 (2H, m), 4.4 (1H, d), 4.5 (1H, d), 7.4 (1H, s), 9.9 (1H, s).

### Example 3A

### 2-(2,5-dioxoimidazolidin-4-yl)-N-{4-[(2-methylquinolin-4-yl)methoxy]phenyl}ethanesulphonamide

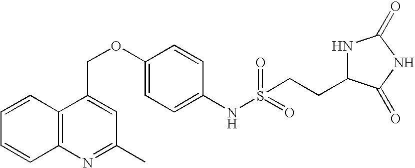

An analogous method to that used in example 1A was used except that [4-methyl-2,5-dioxoimidazolidin-4-yl]methanesulphonyl chloride was replaced with 2-(2,5-dioxoimidazolidin-4-yl)ethanesulphonyl chloride to afford 2-(2,5-dioxoimidazolidin-4-yl)-N-{4-[(2-methylquinolin-4-yl)methoxy]phenyl}ethanesulphonamide as an off white solid; NMR 1.81-1.94 (1H, m), 2.02-2.05 (1H, m), 2.65 (3H, s), 3.02-3.18 (2H, m), 4.07-4.13 (1H, m), 5.56 (2H, s), 7.08-7.20 (4H, m), 7.52-7.60 (2H, m), 7.69-7.76 (1H, m), 7.92-7.97 (2H, m), 8.09 (1H, d); MS 455 (MH+).

The starting material 2-(2,5-dioxoimidazolidin-4-yl)ethanesulphonyl chloride was prepared as follows:
i) Commercially available RS homocystine (0.18 mmol) was suspended in water (25 ml) containing potassium cyanate (1.5 g, 0.2 mmol). The mixture was stirred at 100° C. for 45 min. After partial cooling 10% HCl (10 ml) was added and the mixture stirred at 100° C. for 50 min. The reaction mixture was then placed in the fridge overnight, and resultant crystals were filtered, washed successively with water and dried in vacuo to afford 5-(2-{[2-(2,5-dioxo-4-imidazolidinyl)ethyl]disulphanyl}ethyl)-2,4-imidazolidinedione; MS 319.1 (MH+).
ii) To the suspension of 5-(2-{[2-(2,5-dioxo-4-imidazolidinyl)ethyl]disulphanyl}ethyl)-2,4-imidazolidinedione (6.9 mmol) in a mixture of AcOH (25 ml) and water (2 ml), stirred vigorously and cooled to 0° C., was bubbled chlorine gas for 15 min (until all precipitate dissolved) at maximum temperature of 5° C. After this, stirring was continued for 15 min, and the mixture evaporated to a small volume in vacuo (maximum temperature 30° C.), dissolved in DCM (50 ml), shaken carefully with saturated NaHCO₃ (ca 25 ml) and then with 10% sodium thiosulphate, dried, evaporated and crystallised from THF-hexane (Lora-Tamayo, M. et al, 1968, An. Quim., 64(6):591-606); to afford 2-(2,5-dioxo-4-imidazolidinyl)ethanesulphonyl chloride; ¹H NMR: δ 2.55 (m, 1.1H), 2.65 (m, 1.8H), 2.70 (m, 1H), 4.55 (m, 1H).

### Example 4A

### N-{4-[(2,5-dimethylbenzyl)oxy]phenyl}-1-(4methyl-2,5-dioxoimidazolidin-4-yl)methanesulphonamide

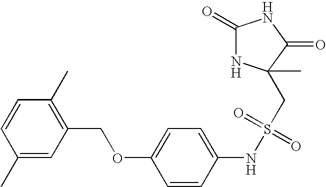

An analogous method to that described in example 1A was used except that 4-((2-methylquinolin-4-yl)methoxy)aniline was replaced with {4-[(2,5-dimethylbenzyl)oxy]phenyl}amine to afford N-{4-[(2,5-dimethylbenzyl)oxy]phenyl}-1-(4-methyl-2,5-dioxoimidazolidin-4-yl)methanesulphonamide as a white solid.

The starting material {4-[(2,5-dimethylbenzyl)oxy]phenyl}amine was prepared as follows:
i) To a stirring solution of tert-butyl (4-hydroxyphenyl)carbamate (CAS registry number 54840-15-2) (2.08 g) under argon and in dimethylacetamide (15 ml) at RT was added sodium hydride (60% dispersion in mineral oil, 44 mg) followed by 2,5-dimethylbenzyl chloride (0.13 ml). After 2 h the reaction mixture was partitioned between 50% aqueous brine (20 ml) and EtOAc (30 ml) and combined organics were dried (sodium sulphate), concentrated in vacuo, purified by chromatography on a 20 g silica gel isolute eluting with 10-20% EtOAc/hexane gradient to give tert-butyl {4-[(2,5-dimethylbenzyl)oxy]phenyl}carbamate as a white solid (2.9 g); NMR δ 1.53 (s, 9H), 2.35 (s, 6H), 4.97 (s, 2H), 6.33 (bs, 1H), 6.93 (d, 2H), 7.02-7.15 (m, 3H), 7.28 (d, 2H); M+Na 350.4, MS 326 (MH−).
ii) Tert-butyl {4-[(2,5-dimethylbenzyl)oxy]phenyl}carbamate was added to 4M HCl in dioxane (20 ml) at RT. After 2 h the reaction mixture was concentrated in vacuo to yield a beige solid, filtered from DCM/diethyl ether (1:1, 20 ml) to give {4-[(2,5-dimethylbenzyl)oxy]phenyl}amine hydrochloride as a white solid (2.17 g). This was used directly in the final step without further purification. A small sample (200 mg) was taken up in EtOAc (5 ml) and the pH adjusted to 7 with a saturated solution of sodium bicarbonate, the organic extract was dried (sodium sulphate) and concentrated in vacuo to give a brown oil. This was purified by chromatography on a 10 g silica gel isolute eluting with 10-30% EtOAc/hexane gradient to give {4-[(2,5-dimethylbenzyl)oxy]phenyl}amine as a brown waxy solid (85 mg); NMR δ 2.33 (s, 6H), 3.42 (bs, 2H), 4.92 (s, 2H), 6.65 (d, 2H), 6.9 (d, 2H), 7.03 (d, 2H), 7.09 (d, 2H), 7.21 (d, 1H); MH 228 (MH+).

### Example 5A

### N-{4-[(2,5-dimethylbenzyl)oxy]phenyl}-1-(4-ethyl-2,5-dioxoimidazolidin-4-yl)methanesulphonamide

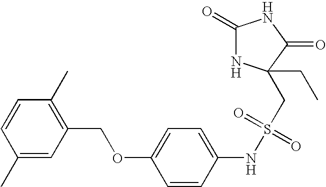

An analogous method to that described in example 2A was used except that 4-((2-methylquinolin-4-yl)methoxy)aniline was replaced with {4-[(2,5-dimethylbenzyl)oxy]phenyl}amine (example 4A step ii)) to afford N-{4-[(2,5-dimethylbenzyl)oxy]phenyl}-1-(4-ethyl-2,5-dioxoimidazolidin-4-yl)methanesulphonamide as a white solid; NMR δ 0.72 (s, 3H), 1.60 (m, 2H), 2.25 (s, 6H), 3.32 (dd, 2H), 4.98 (s, 2H), 6.96 (d, 2H), 7.01-7.10 (m, 2H), 7.13 (d, 2H), 7.22 (s, 1H), 8.01 (s, 1H), 9.55 (s, 1H), 10.71 (s, 1H); MS 430.3 (MH−).

### Example 6A

### N-methyl-1-(4-methyl-2,5-dioxoimidazolidin-4-yl)-N-{4-[(2-methylquinolin-4-yl)methoxy]phenyl}methanesulphonamide (trifluoroacetic acid salt)

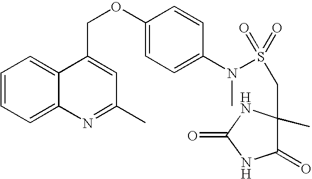

N-methyl-4-[(2-methylquinolin-4-yl)methoxy]aniline (67 mg), (4-methyl-2,5-dioxoimidazolidin-4-yl)methanesulphonyl chloride (example 1A) (82 mg) and triethylamine (67 μl) were stirred under argon in DCM (10 ml) for 16 h. The mixture was washed with water (20 ml), dried (MgSO₄), concentrated in vacuo and purified by prep-HPLC, eluting with a gradient of 5-30% acetonitrile/water to give N-methyl-1-(4-methyl-2,5-dioxoimidazolidin-4-yl)-N-{4-[(2-methylquinolin-4-yl)methoxy]phenyl}methanesulphonamide as a white solid (30 mg); NMR (DMSO-d₆) δ 1.25 (s, 1H), 1.33 (s, 3), 2.90 (s, 3H), 3.20 (s, 3H), 3.30 (m, 1H), 3.65 (m, 1H), 5.80 (s, 2), 7.22 (m, 2H), 7.42 (m, 2H), 7.80 (m, 1H), 7.90 (s, 1H), 8.00 (m, 2H), 8.13 (m, 1H), 8.30 (m, 1H), 10.70 (s, 1H); MS 469 (MH⁺).

The starting material N-methyl-4-[(2-methylquinolin-4-yl)methoxy]aniline was prepared as follows:
i) A mixture of formic acid (1.3 ml) and pentafluorophenol (5.52 g) in DCM (50 ml) was cooled to 0° C. and stirred under argon. To this mixture was added dropwise a solution of 1,3-dicyclohexylcarbodiimide (7.4 g) in DCM (20 ml) and the mixture was stirred at ambient temperature for 90 min. The precipitate that formed was filtered and the filtrate was concentrated in vacuo, redissolved in diethyl ether (50 ml) and washed with saturated sodium bicarbonate solution (2×50 ml), dried (MgSO₄), concentrated in vacuo, redissolved in DCM (20 ml) and added to a solution of {4-[(2-methylquinolin-4-yl)methoxy]phenyl}amine (example 1A, 990 mg) in DCM (50 ml). This mixture was stirred for 16 h and the precipitate that formed was filtered, washed with DCM and dried under vacuum to give {4-[(2-methylquinolin-4-yl)methoxy]phenyl}formamide as a white solid (575 mg); MS 293 (MH⁺).
ii) {4-[(2-methylquinolin-4-yl)methoxy]phenyl}formamide (575 mg) was dissolved in dry THF (20 ml) and stirred under argon at 0° C. A 1M solution of lithium aluminium hydride in THF (2.36 ml) was then added dropwise maintaining the temperature below 5° C., and the mixture was stirred for 2 h. Saturated sodium bicarbonate solution (2 ml) was added and mixture stirred for 5 min, then partitioned between EtOAc (50 ml) and water (50 ml). The organic phase was washed with water (50 ml), dried (MgSO₄) and concentrated in vacuo to give N-methyl-4-[(2-methylquinolin-4-yl)methoxy]aniline as a yellow solid (230 mg); NMR (DMDO-d6) δ 2.65 (m, 6H), 5.20 (m, 1H), 5.45 (s, 2H), 6.50 (d, 2H), 6.92 (d, 2H), 7.52 (m, 1H), 7.55 (m, 1H), 7.74 (m, 1H), 7.95 (m, 1H), 8.10 (m, 1H); MS 279 (MH⁺).

### Example 7A

### N-{4-[(2-methylquinolin-4-yl)methoxy]phenyl}-2,4-dioxo-1,3-diazaspiro[4.5]decane-6-sulphonamide

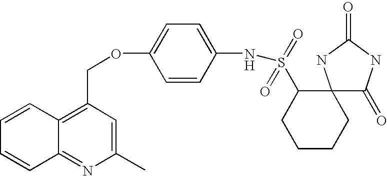

2,4-dioxo-1,3-diazaspiro[4.5]decane-6-sulphonyl chloride (104 mg) was added to a stirred solution 4-[(2-methylquinolin-4-yl)methoxy]aniline (example 1A, 102 mg) in DMSO (2 ml). Triethylamine (0.11 ml) and 4-dimethylaminopyridine (10 mg) were added and the mixture was stirred for 18 h at 20° C. and then for 3 h at 60° C. The mixture was cooled and saturated KH₂PO₄ (5 ml) and water (2 ml) added; a solid formed upon stirring. The solid was filtered off, washed with water, dried, dissolved in a minimum volume of DCM and purified by eluting from a silica column in MeOH/DCM mixtures affording the title compound as a solid. (78 mg); ¹H NMR (DMSOd6) 1.0-2.4 (m, 8H), 2.65 (s, 3H), 4.05 (m, 1H), 5.55 (s, 2H), 7.1, 7.2 (d, d, 4H), 7.55 (s, 1H), 7.6 (m, 1H), 7.65 (m, 1H), 7.95 (d, 1H), 8.1 (d, 1H), 8.65 and 7.75 (s, s 2:3, 1H), 9.7 (s, 1H), 10.5 and 10.6 (s, s, 2:3, 1H); MS (ES)+495.1 (M+H)⁺(ES)⁻493.1 (M−H)⁻.

The starting material 2,4-dioxo-1,3-diazaspiro[4.5]decane-6-sulphonyl chloride was prepared as a mixture of isomers as follows:
i) A solution of 2-benzylthiocyclohexanone (2.56 g) (J. C. S. Perkin 1 1988, 817-821) in EtOH (21 ml) was added to a solution of potassium cyanide (5.98 g), ammonium chloride (7.39 g) and ammonium carbonate (17.68 g) in water (7 ml). The mixture was heated in a microwave apparatus for 5 h at 100° C., cooled and the mixture concentrated. Water was added and the mixture extracted twice with EtOAc. The combined EtOAc extracts were washed with brine, dried over magnesium sulphate, and evaporated to afford 6-(benzylthio)-1,3-diazaspiro[4.5]decane-2,4-dione (2.87 g) as a solid; NMR DMSOd6 1.1-1.9 (m, 8H), 2.65 2.8 (m, m 1:9, 1H), 3.8-3.9 (d, d, 2H), 7.2-7.4 (m, 5H), 7.9, 8.4 (s, s, 1:9, 1H), 10.8 (s, 1H); MS 291.2 (MH+), 289.2 (MH−).
ii) 6-(benzylthio)-1,3-diazaspiro[4.5]decane-2,4-dione (290 mg) was suspended in acetic acid (2 ml) and water (0.2 ml), cooled to 10-15° C. and chlorine was bubbled into the mixture. The solid dissolved after a few minutes and a precipitate formed; the mixture was stirred at 20° C. for 90 min, then evaporated to dryness and azeotroped with toluene (2×5 ml). The residue was stirred with isohexane (10 ml) at 40° C., cooled and filtered affording 2,4-dioxo-1,3-diazaspiro[4.5]decane-6-sulphonyl chloride (216 mg) as a white solid; NMR DMSOd6 1.4-2.1 (m, 8H), 2.3 (d, 1H), 4.4 4.65 (m, m, 1:7, 1H), 8.15 8.9 (s, s, 1:7 1H), 10.8 10.9 (s, s, 1:7 1H).

### Example 8A

### N-{4-[(2-methylquinolin-4-yl)methoxy]phenyl}-2,4-dioxo-1,3-diazaspiro[4.4]nonane-6-sulphonamide

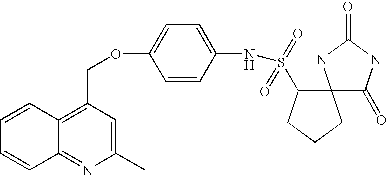

An analogous method to that used in example 7A was used except that 2,4-dioxo-1,3-diazaspiro[4.5]decane-6-sulphonyl chloride was replaced with 2,4-dioxo-1,3-diazaspiro[4.4]nonane-6-sulphonyl chloride to afford N-{4-[(2-methylquinolin-4-yl)methoxy]phenyl}-2,4-dioxo-1,3-diazaspiro[4.4]nonane-6-sulphonamide as a white solid; NMR DMSOd6 1.6-2.2 (m, 6H), 2.65 (s, 3H), 3.5 3.65 (t, t, 1:1, 1H), 5.55 (s, 2H), 7.0-7.2 (s, 4H), 7.5-7.6 (m, 2H), 7.7 (t, 1H), 7.7 8.4 (s, s, 1:1, 1H), 7.95 (d, 1H), 8.1 (d, 1H), 9.6 (s, 1H), 10.6 10.7 (s, s, 1:1, 1H); MS 481.1 (MH+), MS 479.1 (MH−).

The starting material 2,4-dioxo-1,3-diazaspiro[4.4]nonane-6-sulphonyl chloride was prepared by an analogous method to that described in example 7A using steps i) and ii) for the preparation of 2,4-dioxo-1,3-diazaspiro[4.5]decance-6-sulphonyl chloride except that 2-benzylthiocyclopentane was used instead of 2-benzylthiocyclohexane in step i) to yield 6-(benzylthio)-1,3-diazaspiro[4.4]nonane-2,4-dione; NMR DMSOd6 1.5-2.1 (m, 6H), 3.1 3.2 (m, m, 3:7, 1H), 3.7 3.85 (s, s, 7:3, 2H), 7.2-7.4 (m, 5H), 7.9 8.3 (s, s, 3:7, 1H), 10.7 10.8 (s, s, 3:7, 1H); MS 275.2 (MH−) and from step ii) 2,4-dioxo-1,3-diazaspiro[4.4]nonane-6-sulphonyl chloride; NMR DMSOd6 1.8-2.5 (m, 6H), 4.7 4.8 (t, t, 0.33H, 0.66H) 8.15 8.7 (s, s, 0.33H, 0.66H), 10.9 11.5. (s, s, 0,33H, 0.66H).

### Example 9A

### N-{4-[(2-methylquinolin-4-yl)methoxy]phenyl}-2,4-dioxo-7-oxa-1,3-diazaspiro[4.4]nonane-9-sulphonamide

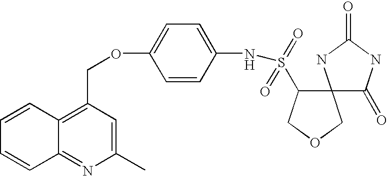

An analogous method to that used in example 7A was used except that 2,4-dioxo-1,3-diazaspiro[4.5]decane-6-sulphonyl chloride was replaced with 2,4-dioxo-7-oxa-1,3-iazaspiro[4.4]nonane-9-sulphonyl chloride to afford N-{4-[(2-methylquinolin-4-yl)methoxy]phenyl}-2,4-dioxo-7-oxa-1,3-diazaspiro[4.4]nonane-9-sulphonamide as a white solid; NMR DMSOd6 2.55 2.65 (s, s, 1:3, 2H), 3.74.1 (m, 5H), 5.55 5.65 (s, s, 3:1, 2H), 7.1-7.2 (m, 4H), 7.25-7.31 (m, 2H), 7.7 (t, 1H), 7.95 (d, 1H), 8.1-8.2 8.6 (s, s 3:1, 2H), 9.8 (s, 1H), 10.8 10.95 (s, s, 3:1, 1H); MS 483.2 (MH+), MS 481.1 (MH−).

The starting material 2,4-dioxo-7-oxa-1,3-diazaspiro[4.4]nonane-9-sulphonyl chloride was prepared as follows:
i) Benzylmercaptan (1.15 g) was added slowly to a stirred solution of 4-bromodihydrofuran-3 (2H)-one (1.5 g) (J. Org. Chem. (1998), 63(8), 2613-2618) and triethylamine (0.91 g) in diethyl ether (15 ml) at 0 to 5° C. The mixture was stirred at 20° C. for 18 h, ether (30 ml) was added, the solution was washed with 2N NaOH, 2N HCl and water, dried and evaporated. The residue was chromatographed on silica (20 g) in MeOH/DCM mixtures affording 4-benzylthiotetrahydrofuran-3-one as a yellow oil (0.57 g); NMR CDCl₃ 3.15 (dd, 1H), 3.8 (d, 1H), 3.85-4.0 (m, 3H), 4.15 (d, 1H), 4.3 (d, 1H), 7.2-7.3 (d, 5H).
ii) An analogous method to that used in example 7A steps i) and ii) was used to prepare 2,4-dioxo-7-oxa-1,3-diazaspiro[4.4]nonane-9-sulphonyl chloride except that in step i) 2-benzylthiocyclohexane was replaced with 4-benzylthiotetrahydrofuran-3-one, a microwave was not used and the mixture was heated at 55° C. for 6 h and the product was purified by chromatography on silica in MeOHJ/DCM mixtures to yield 9-(benzylthio)-7-oxa-1,3-diazaspiro[4.4]nonane-2,4-dione; NMR DMSOd6 3.14.1 (m, 7H), 6.1, 6.7 (s, s, 3:2, 1H), 7.2-7.3 (m, 5H), 8.7 (s, 1H) and step ii) yielded 2,4-dioxo-7-oxa-1,3-diazaspiro[4.4]nonane-9-sulphonyl chloride; NMR DMSOd6 3.4-5.3 (m, 5H), 7.3 8.5 (s, s, 2:1, 1H), 11.15 11.23 (s, s, 2:1, 1H).
   Compounds of Formula (1B)

### Example 1B

### 5-[1-({4-[(2-methylquinolin-4-yl)methoxy]phenyl}sulphonyl)pyrrolidin-2-yl]imidazolidine-2,4-dione

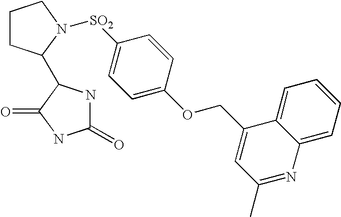 1-{4-[(2-Methylquinolin-4-yl)methoxy]sulphonyl}pyrrolidin-2-ylcarbaldehyde (prepared as described below) (198 mg, 0.48 mmol) was stirred in ethanol (5 ml) and water (4 ml). Ammonium carbonate (232 mg, 2.41 mmol) was added followed by potassium cyanide (38 mg, 0.58 mmol) and the reaction mixture was heated at 60 to 65° C. for 5 h. The mixture was then concentrated in vacuo, diluted with water (15 ml) and extracted with EtOAc (3×15 ml). The combined organic extracts were washed with brine (15 ml), dried (MgSO₄), filtered and evaporated. The residue was purified by column chromatography (20 g silica bond elut, eluent 0-4% MeOH in DCM) to give the product 5-[1-({4-[(2-methylquinolin-4-yl)methoxy]phenyl}sulphonyl)pyrrolidin-2-yl]imidazolidine-2,4-dione as a mixture of 4 diastereoisomers (77 mg, 0.16 mmol). NMR: 1.20-1.82 (m, 4H), 2.66 (s, 3H), 3.15-3.41 (m, 2H), 3.73-3.81 (m, A 1H), 3.81-3.89 (m, B 1H), 4.15 (d, B 1H), 4.47 (s, A 1H), 5.72 (s, 2H), 7.35 (d, B 2H), 7.40 (d, A 2H), 7.55 (s, 1H), 7.58 (t, 1H), 7.74 (t, 1H), 7.82 (d, 2H), 7.88 (s, B 1H), 7.95 (d, 1H), 8.10 (d, 1H), 8.25 (s, A 1H), 10.66 (s, B 1H), 10.76 (s, A 1H); MS (M+H) 481.

The starting material 1-({4-[(2-methylquinolin-4-yl)methoxy]phenyl}sulphonyl)pyrrolidin-2-ylcarbaldehyde was prepared as described below:
i) To a stirred suspension of 2-methylquinolin-4-ylcarboxylic acid (4 g, 21.4 mmol) in THF (100 ml) at RT was added lithium aluminium hydride (21.4 ml, 1.0M solution in THF, 21.4 mmol) dropwise over 20 min. After 16 h water (4 ml) was added cautiously followed by 2N NaOH (4 ml) and water (12 ml). The resulting gelatinous precipitate was filtered off and washed with THP. DCM (200 ml) was added to the filtrate and partitioned with saturated NaHCO₃ (2×75 ml). The organic layer was dried (MgSO₄), concentrated, triturated with DCM and filtered to give 2-methylquinolin-4-ylmethanol as a white powder (858 mg, 5 mmol). The mother liquours were purified by chromatography (20 g silica bond elute, eluent 0→5% EtOH/DCM) to give a further 610 mg of product (3.5 mmol). NMR: 2.6 (s, 3H), 5.0 (d, 2H), 5.5 (t, 1H), 7.4 (s, 1H), 7.5 (t, 1H), 7.7 (t, 1H) and 7.9 (m, 2H); MS: 174.
ii) To a suspension of 2-methylquinolin-4-ylmethanol (100 mg, 0.58 mmol) in DCM (5 ml) at RT was added triethylamine (0.24 ml, 1.74 mmol). The reaction mixture was then cooled to 0° C. and methanesulphonylchloride (0.05 ml, 0.64 mmol) was added dropwise. After 10 min the reaction mixture was concentrated, EtOAc (20 ml) was added and the organic layer partitioned with brine (10 ml), dried (MgSO₄), concentrated and purified by chromatography (10 g silica bond elute, eluent 5% MeOH/DCM) to give 2-methylquinolin-4-ylmethyloxysulphonylmethane (110 mg, 0.44 mmol). NMR: 2.7 (s, 3H), 3.35 (s, 3H), 5.75 (s, 2H), 7.5 (s, 1H), 7.6 (t, 1H), 7.75 (t, 1H), 8.0 (m, 2H): MS: 252.
iii) 4-Hydroxythiophenol (4.448 g) was dissolved in MeOH (100 ml). The solution was stirred at RT and water (35 ml) was added, followed by sodium perborate tetrahydrate (10.86 g). After 1 h the reaction mixture was partitioned between 50% brine (100 ml) and EtOAc (2×200 ml) and the combined organics were dried (sodium sulphate) and concentrated in vacuo to give 4-hydroxythiophenol disulphide as a white waxy solid (4.28 g); NMR δ 6.75 (d, 4H), 7.25 (d, 4H), 9.75 (s, 2H); MS 249.59 (MH−).
iv) 4-Hydroxythiophenol disulphide (4.27 g) and 4-methanesulphonyloxymethyl-2-methylquinoline (example 1B step ii)) (8.86 g) were dissolved in DMF (150 ml). Potassium carbonate (14.15 g) was added and the mixture was stirred at 50° C. under an atmosphere of argon for 4 h. The suspension was allowed to cool to RT and partitioned between 50% brine (150 ml) and EtOAc (2×300 ml). The combined organic washings were dried (sodium sulphate) and concentrated and the residue triturated with cold MeOH to give the desired product as an off-white solid (4.58 g). Further product was obtained by silica column chromatography of the mother liquors using a 25%-75% EtOAc/isohexane gradient over 50 min as eluent giving 4-(2-methylquinolin-4-ylmethyloxy)thiophenol disulphide 7.66 g; NMR o 2.65 (s, 6H), 5.6 (s, 4H), 7.15 (d, 4H), 7.5 (m, 8H), 7.7 (t, 2H), 7.95 (d, 2H), 8.1 (d, 2H); MS 561.39 (MH+).
v) 4-(2-methylquinolin-4-ylmethyloxy)thiophenol disulphide (7.5 g) was cooled to 5° C. in a mixture of acetic acid (170 ml) and water (20 ml). Chlorine gas was bubbled through the mixture for 20 min. The mixture was then stirred at ambient temperature for a further hour before removing the solvent by evaporation under reduced pressure and azeotroping with toluene. 4-[(2-methylquinolin-4-yl)methoxy]benzenesulphonyl chloride hydrochloride was obtained as a yellow solid. NMR DMSO-d6 3.0 (3H, s), 5.9 (2H, s), 7.6 (2H, m), 7.9 (1H, m), 8.0-8.1 (2H, m), 8.35 (1H, m), 8.45 (1H, m); MS 348 (MH+)
vi) 4-[2-Methylquinolin-4-yl)methoxy]benzenesulphonyl chloride hydrochloride (552 mg, 1.44 mmol) was stirred in DCM (20 ml) under argon. Diisopropylethylamine (275 μl, 1.58 mmol) was added followed by 2-(D)-pyrrolidinylmethanol (142 μl, 1.44 mmol) and stirred at RT for 3 h. A further portion of 2-(D)-pyrrolidinemethanol (30 μl, 0.304 mmol) was added and stirring continued for 1.5 h. The DCM solution was then washed with water (15 ml), dried (MgSO₄), filtered and evaporated to give [1-({4-[(2-methylquinolin-4-yl)methoxy]phenyl}sulphonyl)pyrrolidin-2-yl]methanol (515 mg, 1.25 mmol) as a pale yellow glassy solid. NMR: 1.20-1.86 (m, 4H), 2.67 (s, 3H), 2.97-3.65 (m, 5H), 4.74-7.83 (m, 1H), 5.71 (s, 2H), 7.34 (d, 2H), 7.55 (s, 1H), 7.58 (t, 1H), 7.74 (t, 1H), 7.79 (d, 2H), 7.96 (d, 1H), 8.11 (d, 1H); MS 413 (MH+)
vii) [1-({4-[(2-Methylquinolin-4-yl)methoxy]phenyl}sulphonyl)pyrrolidin-2-yl]methanol (250 mg, 0.606 mmol) was stirred in DCM (12 ml). Dess-Martin periodinane (2.06 ml of a 15% wt solution in DCM, 0.727 mmol) was added dropwise and the solution was stirred for 2 min before addition of 1 drop of water. Stirring was continued at RT for 1 h, then the DCM solution was washed with 1M aqueous NaOH solution (10 ml), washed with water (10 ml), dried (MgSO₄), filtered and evaporated to give the product 1-({4-[(2-methylquinolin-4-yl)methoxy]phenyl}sulphonyl)pyrrolidin-2-ylcarbaldehyde as a pale brown solid (202 mg, 0.492 mmol). NMR: 1.41-2.05 (m, 4H), 2.67 (s, 3H), 3.11-3.22 (m, 1H), 3.36-3.48 (m, 1H), 3.89-3.96 (m, 1H), 5.74 (s, 2H), 7.40 (d, 2H), 7.57 (s, 1H), 7.61 (t, 1H), 7.77 (t, 1H), 7.84 (d, 2H), 7.99 (d, 1H), 8.12 (d, 1H), 9.55 (s, 1H); MS 411 (MH+).

### Example 2B

### 5-(1-{4-[(2-methylquinolin-4-yl)methoxy]benzoyl}pyrrolidin-2-yl)imidazolidine-2,4-dione

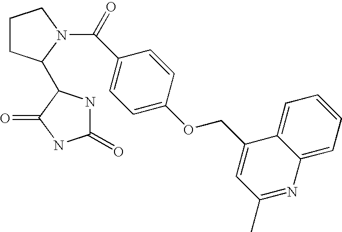

An analogous method to that described in example 1B was used to yield a mixture of 4 diastereoisomers except that 1-({4-[(2-methylquinolin-4-yl)methoxy]phenyl}sulphonyl)pyrrolidin-2-ylcarbaldehyde was replaced with 1-{4-[(2-methylquinolin-4-yl)methoxy]benzoyl}pyrrolidin-2-ylcarbaldehyde (prepared as described below). The product was purified by chromatography (10 g silica bond elut, eluent 0-4% MeOH in DCM). Fraction 1: (A:B 5:1) NMR: 1.46-2.16 (m, 4H), 2.68 (s, 3H), 3.30-3.59 (m, 2H), 4.35-4.50 (m, 1H+ B 1H), 4.76 (s, A 1H), 5.67 (s, 2H), 7.22 (d, 2H), 7.55 (d, B 2H), 7.58 (s, 1H), 7.60 (t, 1H), 7.66 (d, A 2H), 7.76 (t, 1H), 7.99 (d, 1H), 7.99 (s, B 1H), 8.12 (d, 1H), 8.21 (s, A 1H), 10.60 (s, B 1H), 10.74 (s, A 1H); MS 445 (MH+) Fraction 2: (A:B 4:3) MS 445 (MH+).

The starting material 1-{4-[(2-methylquinolin-4-yl)methoxy]benzoyl}pyrrolidin-2-ylcarbaldehyde was prepared as described below:
i) (2-methylquinolin-4-yl)methanol (example 1B step i), 12.04 g) was suspended in DCM (300 ml). DMF (1 ml) added, followed by the dropwise addition of thionyl chloride (5.59 ml), keeping temperature below 30° C. The reaction mixture stirred for 16 h at ambient temperature, then filtered. The precipitate was washed further with DCM (2×50 ml) and dried under vacuum to give 4-chloromethyl-2-methylquinoline as a cream solid (8.79 g); NMR DMSO-d6 δ 2.95 (m, 3H), 5.42 (m, 2H), 7.90 (m, 1H), 8.00 (s, 1H), 8.05 (m, 1H), 8.40 (m, 2H); MS 192 (MH⁺).
ii) 4-(chloromethyl)-2-methylquinoline (8.79 g), methyl 4-hydroxybenzoate (6.96 g), sodium iodide (6.87 g) and potassium carbonate (63.18 g) were stirred in acetone (500 ml) at 70° C., under reflux, for 16 h. The reaction mixture was allowed to cool to ambient temperature and filtered. Filtrate was concentrated in vacuo and dried under vacuum to give methyl 4-[(2methylquinolin-4-yl)methoxy]benzoate as an off-white solid (12.14 g); NMR DMSO-d6 δ 2.65 (s, 3H), 3.82 (s, 3H), 5.70 (s, 2M), 7.25 (m, 2H), 7.55 (m, 2H), 7.75 (m, 1H), 7.95 (m, 3H), 8.10 (m, 1H); MS 308 (MH⁺.
iii) Methyl 4-[(2-methylquinolin-4-yl)methoxy]benzoate (12.14 g) was dissolved in THF (85 ml). 1M aqueous NaOH (85 ml) was then added and reaction mixture stirred at 90° C., under reflux, for 16 h. The mixture allowed to cool to ambient temperature and neutralised to pH7 with 1M aqueous HCl. The resulting precipitate was filtered, washed with water and acetonitrile, then dried under vacuum to give 4-[(2-methylquinolin-4-yl)methoxy]benzoic acid as an off-white solid (10.08 g); NMR δ(CD₃SOCD₃) 2.65 (s, 3H), 5.70 (s, 2H), 7.22 (d, 2H), 7.55 (m, 2H), 7.75 (m, 1H), 7.95 (m, 3H), 8.10 (m, 1H), 12.60 (s, 1H); LCMS M/z (+) 294 (MH⁺.
iv) 4-[(2-Methylquinolin-4-yl)methoxy]benzoic acid (500 mg, 1.70 mmol) was stirred in DCM (25 ml) with (R)-2-pyrrolidinylmethanol (185 μl, 1.8 mmol), PS-DMAP (2.30 g, loading 1.48 mmol/g) and EDCI (359 mg, 1.87 mmol). After 3 h the solution was filtered, washed through with DCM (10 ml) and the filtrate was washed with water (15 ml). The organic layer was then separate and evaporated in vacuo, before purification by column chromatography (10 g silica bond elut, eluent 0-3% MeOH in DCM) to give the product 1-{4-[(2-methylquinolin-4-yl)methoxy]benzoyl}pyrrolidin-2-ylmethanol as a colourless gum (176 mg, 0.468 mmol); NMR: 1.59-2.01 (m, 4H), 2.67 (s, 3H), 3.25-3.70 (m, 4H), 4.06-4.21 (m, 1H), 4.70-4.80 (m, 1H), 5.66 (s, 2H), 7.18 (d, 2H), 7.52 (d, 2H), 7.56 (s, 1H), 7.60 (t, 1H), 7.75 (t, 1H), 7.98 (d, 1H), 8.13 (d, 1H); MS 377 (MH+).
v) 1-{4-[(2-Methylquinolin-4-yl)methoxy]benzoyl}pyrrolidin-2-ylcarbaldehyde was prepared from (1-{4-[(2-methylquinolin-4-yl)methoxy]benzoyl}pyrrolidin-2-ylmethanol as described for example 1B step vii) and used crude for subsequent reaction. MS 373 (MH−)

### Example 3B

### 5-[1-({4-[(2-methylquinolin-4-yl)methoxy]phenyl}sulphonyl)piperidin-2-yl]imidazolidine-2,4-dione

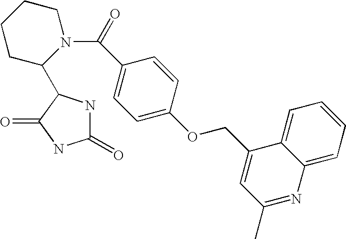

An analogous method to that described in example 1B was used to obtain a mixture of 4 diastereomers except that 1-({4-[(2-methylquinolin-4-yl)methoxy]phenyl}sulphonyl)pyrrolidin-2-ylcarbaldehyde was replaced with 1-({4-[(2-methylquinolin-4-yl)methoxy]phenyl}sulphonyl)piperidin-2-ylcarbaldehyde (prepared as described below). The product was purified by chromatography (10 g silica bond elut, eluent 0-3% MeOH in DCM); Fraction 1: (A:B 5:3) NMR: 0.80-2.03 (m, 6H), 2.67 (s, 3H), 3.07-3.20 (m, 1H), 3.62-3.78 (m, 1H), 3.97-4.12 (m, 1H), 4.31-4.44 (m, 1H), 5.74 (s, 2H), 7.30-7.39 (m, 2H), 7.56 (s, 1), 7.60 (t, 1H), 7.76 (t, 1H), 7.85 (d, 2H), 7.88 (s, A 1H), 7.99 (d, 1H), 8.12 (d, 1H), 8.14 (s, B 1H), 10.67 (s, A 1H), 10.75 (s, B 1H); MS 495 (MH+).

The starting material 1-({4-[(2-methylquinolin-4-yl)methoxy]phenyl}sulphonyl)piperidin-2-ylcarbaldehyde was prepared as described below:
i) [1-({4-[(2-methylquinolin-4-yl)methoxy]phenyl}sulphonyl)piperidin-2-yl]methanol was prepared by an analogous method to that described in as for example 1B step vi) from 4-[(2-methylquinolin-4-yl)methoxy]benzenesulphonyl chloride hydrochloride except that 2-(D)-pyrrolidinylmethanol was replaced with 2-piperidinylmethanol. The crude product was used immediately without further purification in the subsequent reaction. MS 427 (MH+).
ii) 1-({4-[(2-methylquinolin-4-yl)methoxy]phenyl}sulphonyl)piperidin-2-ylcarbaldehyde was prepared by an analogous method to that described in example 1B step vii) except that [1-({4-[(2-methylquinolin-4-yl)methoxy]phenyl}sulphonyl)pyrrolidin-2-yl]methanol was replaced with [1-({4-[(2-methylquinolin-4-yl)methoxy]phenyl}sulphonyl)piperidin-2-ylmethanol; NMR 1.15-1.52 (m, 5H), 1.90-2.02 (m, 1H), 2.68 (s, 3H), 3.08-3.37 (m, 2H), 4.15-4.21 (m, 1H), 5.74 (s, 2H), 7.38 (d, 2H), 7.54-7.65 (m, 2H), 7.72-7.83 (m, 3H), 7.99 (d, 1H), 8.12 (d, 1H), 9.50 (s, 1H); MS 425 (MH+).

### Example 4B

### (5R)-5-methyl-5-[(2R)-1-({4-[(2-methylquinolin-4-yl)methoxy]phenyl}sulphonyl)pyrrolidin-2-yl]imidazoline-2,4-dione

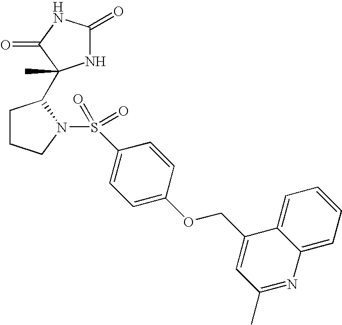

Thionyl chloride (0.85 ml, 12.00 mmol) was added dropwise to methanol (8 ml), stirred and cooled in a water bath. Stirring was continued at RT for 50 min then tert-butyl (2R)-[(4R)-4-methyl-2,5-dioxoimidazolidin-4-yl]pyrrolidin-1-ylcarboxylate (prepared as described below) (133 mg, 0.468 mmol) was added in one portion. After stirring for a further 30 min the solution was evaporated to dryness and re-evaportaed twice with EtOH (2×3 ml), then dried in vacuo. The residual solid was dissolved in DCM (5 ml) under argon, to this was added triethylamine (80 μl, 0.574 mmol) and the mixture was stirred at RT for 10 min. 4-[2-Methylquinolin-4-yl)methoxy]benzenesulphonyl chloride hydrochloride (example 1B step v)) (180 mg, 0.468 mmol) was suspended in DCM (5 ml) and to this was added triethylamine (130 μl, 0.933 mmol); the resulting solution was then added dropwise to the amine solution and stirring continued under argon for 16 h. The solution was diluted with DCM (20 ml) and washed with water (15 ml). The organic layer was evaporated and purified by column chromatography (log silica bond elut, eluent 0-2% MeOH in DCM). Product fractions were evaporated, triturated with ether and collected by filtration to give (5R)-5-methyl-5-[(2R)-1-({4-[(2-methylquinolin-4-yl)methoxy]phenyl}sulphonyl)piperidin-2-yl]imidazoline-2,4-dione as a white solid (113 mg, 0.228 mol). NMR: 1.02-1.15 (m, 1H), 1.23-1.48 (m, 2H), 1.44 (s, 3H), 1.60-1.74 (m, 1H), 2.66 (s, 3H), 3.22-3.50 (m, 2H), 3.95-4.02 (m, 1 μl), 5.74 (s, 2H), 7.36 (d, 2H), 7.55 (s, 1H), 7.60 (t, 1H), 7.76 (t, 1H), 7.84 (d, 2H), 7.99 (d, 1H), 8.07 (s, 1H), 8.12 (d, 1H), 10.80 (s, 1H); MS (M+H) 495.

The starting material tert-butyl (2R)-[(4R)-methyl-2,5-dioxoimidazolidin-4-yl]pyrrolidin-1-ylcarboxylate was prepared as described below:
i) 1-(tert-Butoxycarbonyl)-D-proline (5 g, 23.2 mmol) was dissolved in DCM (120 ml). Triethylamine (3.23 ml, 23.23 mmol) was added and the reaction mixture was stirred rapidly and cooled in an ice-salt bath. Isobutyl chloroformate (2.96 ml, 23.30 mmol) was added dropwise, maintaining the reaction temperature under −5° C. Stirring was continued at this temperature for 15 min, then N,O-dimethylhydroxylamine hydrochloride (2.34 g, 24.00 mmol) was added in one portion, followed by dropwise addition of triethylamine (3.23 ml, 23.23 mmol). Stirring was continued at −5° C. to −10° C. for 1 h then at RT for 1.5 h. The solution was then washed with saturated aqueous NaHCO₃ (40 ml), water (40 ml) and brine (40 ml), then dried (MgSO₄), filtered, evaporated and dried in vacuo to give the product tert-butyl (2R)-2-{[methoxy(methyl)amino]carbonyl}pyrrolidin-1-ylcarboxylate as a syrup (5.4 g, 20.90 mmol), used without further purification.
ii) tert-Butyl (2R)-2-{[methoxy(methyl)amino]carbonyl}pyrrolidin-1-ylcarboxylate (5.40 g, 20.90 mmol) was stirred in THF (70 ml) under argon and the solution cooled to around −10° C. in an ice-salt bath. Methylmagnesium chloride (13.9 ml of a 3M solution in THF, 41.80 mmol) was added dropwise and stirring was continued for 1 h at −10° C., then at RT for 16 h. EtOAc (50 ml) was added with vigorous stirring, followed by 2M aqueous HCl (50 ml). The layers were separated and the aqueous phase was re-extracted with EtOAc (3×40 ml). The combined organic extracts were washed with saturated aqueous NaHCO₃ (80 ml), brine (80 ml), dried (MgSO₄), filtered and evaporated to give the product tert-butyl (2R)-2-acetylpyrrolidin-1-ylcarboxylate (3.40 g, 15.94 mmol) as a pale yellow oil which crystallised on standing in the freezer, and was used without further purification.
iii) tert-Butyl (2R)-2-acetylpyrrolidin-1-ylcarboxylate (2 g, 9.38 mmol) was dissolved in EtOH (20 ml) and to this was added a solution of ammonium carbonate (3.60 g, 37.46 mmol) in water (20 ml), followed by potassium cyanide (1.22 g, 18.73 mmol). The reaction mixture was heated at 80° C. under microwave irradiation for 2 h, cooled and allowed to stand at RT for 48 h, then poured into water (60 ml) and extracted with EtOAc (4×50 ml). The combined organic extracts were washed with brine (50 ml), dried (MgSO₄), filtered and evaporated. The residual foamy solid was recrystallised from tert-butyl methyl ether (60 ml) to give the product tert-butyl (2R)-[(4R)₄-methyl-2,5-dioxoimidazolidin-4-yl]pyrrolidin-1-ylcarboxylate (1.09 g, 3.85 mmol) as a white crystalline solid. The filtrate was evaporated and recrystallised from tert-butyl metyl ether (ca 20 ml) to give a further portion of tert-butyl (2R)-[(4R)-4-methyl-2,5-dioxoimidazolidin-4-yl]pyrrolidin-1-ylcarboxylate (0.586 g, 2.70 mmol); NMR: 1.19 (s, 9H), 1.73-2.06 (m, 3H), 2.32 (bs, 1H), 3.15-3.24 (m, 1H), 3.22 (s, 3H), 3.51 (bs, 1H), 4.11-4.19 (m, 1H), 6.18 (bs, 1H), 7.57 (bs, 1H).
   Other Compounds

### Example 1

### 7-({4-[(2-methylquinolin-4-yl)methoxy]phenyl}sulphonyl)-1,3-diazaspiro[4.6]undecane 2,4-dione

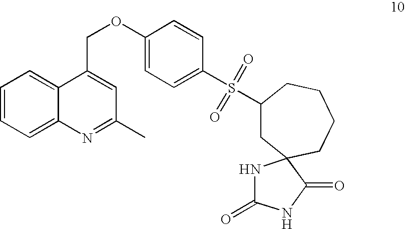

To a solution of 7-({4-[(2-methylquinolin-4-yl)methoxy]phenyl}thio)-1,3-diazaspiro[4.6]undecane-2,4-dione (140 mg) in MeOH (10 ml) at 0° C. was added a suspension of potassium peroxymonosulphate (300 mg) in water (10 ml). The resultant suspension was stirred for 1 h, diluted with water (50 ml) and portioned with DCM (3×80 ml). The combined organic extracts were treated with water (50 ml) and brine (50 ml), dried and concentrated in vacuo. The crude product was chromatographed on silica (10 g) using a 0-10% EtOH/DCM gradient over 50 min as eluent to give 7-({4-[(2-methylquinolin-4-yl)methoxy]phenyl}sulphonyl)-1,3-diazaspiro[4.6]undecane-2,4-dione as a white solid (123 mg); NMR δ 1.4 (m, 7H), 2.0 (m, 4H), 2.8 (s, 3H), 5.8 (s, 2H), 7.5 (m, 2H), 7.8 (m, 4H), 7.9 (s, 1H), 8.1 (d, 1H), 8.3 (d, 1H), 8.4 (s, 1H), 10.6 (m, 1H), diastereoisomeric enrichment approximately 2.2:1; MS 493.95 (MH+).

The starting material 7-({4-[(2-methylquinolin-4-yl)methoxy]phenyl}thio)-1,3-diazaspiro[4.6]undecane-2,4-dione was prepared as follows:
i) To a stirred suspension of 2-methylquinolin-4-ylcarboxylic acid (4 g, 21.4 mmol) in THF (100 ml) at RT was added lithium aluminium hydride (21.4 ml, 1.0M solution in THF, 21.4 mmol) dropwise over 20 min. After 16 h, water (4 ml) was added cautiously followed by 2N NaOH (4 ml) and water (12 ml). The resulting gelatinous precipitate was filtered off and washed with THF. DCM (200 ml) was added to the filtrate and partitioned with saturated NaHCO₃ (2×75 ml). The organic layer was dried (MgSO₄), concentrated, triturated with DCM and filtered to give 2-methylquinolin-4-ylmethanol as a white powder (858 mg, 5 mmol). The mother liquours were purified by chromatography (20 g silica bond elute, eluent 0>5% EtOH/DCM) to give a further 610 mg of product (3.5 mmol); NMR 2.6 (s, 31), 5.0 (d, 2H), 5.5 (t, 1H), 7.4 (s, 1H), 7.5 (t, 1H), 7.7 (t, 1H), 7.9 (m, 2H); MS: 174.
ii) To a suspension of 2-methylquinolin-4-ylmethanol (100 mg, 0.58 mmol) in DCM (5 ml) at RT was added triethylamine (0.24 ml, 1.74 mmol). The reaction mixture was cooled to 0° C. and methanesulphonylchloride (0.05 ml, 0.64 mmol) was added dropwise. After 10 min the reaction mixture was concentrated, EtOAc (20 ml) was added and the organic layer partitioned with brine (10 ml), dried (MgSO₄), concentrated and purified by chromatography (10 g silica bond elute, eluent 5% MeOH/DCM) to give 2-methylquinolin-4-ylmethoxysulphonylmethane (110 mg, 0.44 mmol); NMR 2.7 (s, 3H), 3.35 (s, 3H), 5.75 (s, 2H), 7.5 (s, 1H), 7.6 (t, 1H), 7.75 (t, 1H), 8.0 (m, 2H); MS: 252.
iii) 4-Hydroxythiophenol (4.448 g) was dissolved in MeOH (10 ml). The solution was stirred at RT and water (35 ml) was added followed by sodium perborate tetrahydrate (10.86 g). After 1 h the reaction mixture was partitioned between 50% brine (100 ml) and EtOAc (2×200 ml), the combined organic extracts dried (sodium sulphate) and concentrated in vacuo to give 4-hydroxythiophenol disulphide as a white waxy solid (4.28 g); NMR δ 6.75 (d, 4H), 7.25 (d, 4H), 9.75 (s, 2H); MS 249.59 (MH−).
iv) 4-Hydroxythiophenol disulphide (4.27 g) and 2-methylquinolin-4-ylmethoxysulphonylmethane (8.86 g) were dissolved in DMF (150 ml). Potassium carbonate (14.15 g) was added and the mixture was stirred at 50° C. under argon for 4 h. The suspension was allowed to cool to RT and partitioned between 50% brine (150 ml) and EtOAc (2×300 ml). The combined organic extracts were dried (sodium sulphate), concentrated and the residue triturated with cold MeOH to give the desired product as an off-white solid (4.58 g). Further product was obtained by silica column chromatography of the mother liquors using a 25%-75% EtOAc/isohexane gradient over 50 min as eluent giving 4-(2-methylquinolin-4-ylmethyloxy)thiophenol disulphide (7.66 g); NMR δ 2.65 (s, 6H), 5.6 (s, 4H), 7.15 (d, 4H), 7.5 (m, 8H), 7.7 (t, 2H), 7.95 (d, 2H), 8.1 (d, 2H); MS 561.39 (MH+).
v) 4-(2-Methylquinolin-4-ylmethyloxy)thiophenol disulphide (500 mg) was suspended in acetonitrile (10 ml) and stirred at RT. Water (1 drop) was added followed by tri-n-butylphosphine (0.28 ml). Stirring was continued overnight leaving a clear solution which was concentrated in vacuo and purified by chromatography on a silica gel bond elute using a 30-90% EtOAc/hexane gradient over 50 min as eluent to give 4-(2-methylquinolin-4-ylmethoxy)thiophenol as a white, waxy solid (480 mg); NMR δ 2.75 (s, 3H), 5.2 (s, 1H), 5.6 (s, 2H), 7.05 (d, 2H), 7.3 (d, 2H), 7.6 (m, 2H), 7.8 (t, 1H), 7.95 (d, 1H), 8.1 (d, 1H); MS 280.2 (M−H).
vi) To 4-(2-methylquinolin-4-ylmethoxy)thiophenol (246 mg) stirred at RT in DCM (5111) was added 2-cyclohepten-1-one (110 ml) and triethylamine (0.4 ml). After 2 h the reaction mixture was partitioned between 50% saturated brine (10 ml) and DCM (20 ml). The organic portion was dried, concentrate in vacuo and the residue was purified by chromatography on a silica gel bond elute using a 0-100% EtOAc/hexane gradient over 45 min to give 3-({4-[{2-methylquinolin-4-yl)methoxy]phenyl}thio)cycloheptanone as a colourless oil (310 mg); MS 392.2 (MH+).
vii) 3-({4-[{2-methylquinolin-4-yl)methoxy]phenyl}thio)cycloheptanone (310 mg) was suspended in EtOH (10 ml). Water (10 ml), ammonium carbonate (900 mg) and potassium cyanide (130 mg) were added and the reaction stirred at 65° C. overnight. EtOH was removed in vacuo and the mixture partitioned between water (20 ml) and DCM (2×40 ml). The combined organic extracts were dried (sodium sulphate), concentrated in vacuo and purified on a 20 g silica bond elute using a 0-7.5% EtOH/DCM gradient over 45 min as eluent to give 7-({4-[(2-methylquinolin-4-yl)methoxy]phenyl}thio)-1,3-diazaspiro[4.6]undecane-2,4-dione as a white foam (140 mg); MS 461.96 (MH+).

### Example 2

### 7-({4-[(2-Methylquinolin-4-yl)methoxy]phenyl}sulphonyl)-1,3-diazaspiro[4.5]decane-2,4-dione

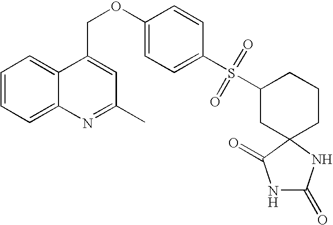

An analogous method to that described in example 1 was used except that the starting material was 7-({4-[(2-methylquinolin-4-yl)methoxy]phenyl}thio)-1,3-diazaspiro[4.5]decane-2,4dione. The crude product was chromatographed on a 10 g silica bond elute using a 0-25% EtOH/DCM gradient over 50 min as eluent to give 7-({4-[(2-methylquinolin-4-yl)methoxy]phenyl}sulphonyl)-1,3-diazaspiro[4.5]decane-2,4-dione as a white solid (75 mg); NMR δ 1.4 (m, 5H), 2.0 (m, 4H), 2.8 (s, 3H), 5.8 (s, 2H), 7.5 (m, 2H), 7.8 (m, 4H), 7.9 (s, 1H), 8.1 (d, 1H), 8.3 (d, 1H), 8.4 (s, 1H), 10.6 (m, 1H), diastereoisomeric enrichment approximately 4.2:1; MS 479.93 (MH+)

The starting material 7-({4-[(2-methylquinolin-4-yl)methoxy]phenyl}thio)-1,3-diazaspiro[4.5]decane-2,4-dione was prepared using an analogous method to that describe in example 1 for the preparation of 7-({4-[(2-methylquinolin-4-yl)methoxy]phenyl}thio)-1,3-diazaspiro[4.6]undecane-2,4-dione except that 2-cyclohexen-1-one was used instead of 2-cyclohepten-1-one in step vi) to yield 7-({4-[(2-methylquinolin-4-yl)methoxy]phenyl}thio)-1,3-diazaspiro[4.5]decane-2,4-dione as a white foam (140 mg); MS 448.0 (MH+).

### Example 3

### 7-({4-[(2-methylquinolin-4-yl)methoxy]phenyl}sulphonyl)-1,3-diazaspiro[4.4]nonane-2,4-dione

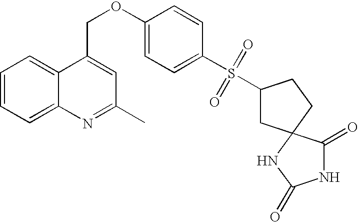

An analogous method to that described in example 1 was used except the starting material was 7-({4-[(2-methylquinolinfyl)methoxy]phenyl}thio)-1,3-diazaspiro[4.4]nonane-2,4-dione (70 mg). The crude product was chromatographed on a 10 g silica bond elute using a 0-20% EtOH/DCM gradient over 45 min as eluent to give 7-({4-[(2-methylquinolin-4-yl)methoxy]phenyl}sulphonyl)-1,3-diazaspiro[4.4]nonane-2,4-dione as a white solid (20 mg); NMR δ 2.0 (m, 6H), 2.7 (s, 3H), 3.9 (m, 1H), 5.7 (s, 2H), 7.4 (m, 2H), 7.5 (m, 2H), 7.6 (m, 1H), 7.7 (m, 2H), 7.9 (m, 2H), 8.1 (m, 1H), 10.6 (m, 1H), diastereoisomeric enrichment approximately 4.6:1; MS 465.89 (MH+).

The starting material 7-({4-[(2-methylquinolin-4-yl)methoxy]phenyl}thio)-1,3-diazaspiro[4.4]nonane-2,4-dione was prepared as follows:
i) 4-(2-Methylquinolin-4-ylmethoxy)thiophenol disulphide (200 mg) (example 1, step iv)) was stirred at RT in acetonitrile (10 ml), water (1 drop) and tri-n-butylphosphine (0.094 ml) were added. Stirring was continued overnight and 2-cyclopenten-1-one (0.101 ml) and triethylamine (0.3 ml) were added. After 2 h the mixture was partitioned between saturated brine (20 ml) and EtOAc (40 ml). The organic portion was concentrated in vacuo and chromatographed on a 20 g silica bond elute using a 30-100% EtOAc/isohexane gradient over min as eluent to give 3-({4-[(2-methylquinolin-4-yl)methoxy]phenyl}thio)cyclopentanone as a colourless oil (240 mg); NMR δ 2.2 (m, 6H), 2.6 (m, obscure, 2H), 2.65 (s, 3H), 3.9 (m, 1H), 7.1 (d, 2H), 7.4 (d, 2H), 7.55 (m, 2H), 7.7 (t, 1H), 7.95 (d, 1H), 8.1 (d, 1H); MS 363.99 (MH+).
ii) 3-({4-[(2-methylquinolin-4-yl)methoxy]phenyl}thio)cyclopentanone (230 mg) was treated using the method given in example 1 step vii) to yield 7-({4-[(2-methylquinolin-4-yl)methoxy]phenyl}thio)-1,3-diazaspiro[4.4]nonane-2,4-dione as a white solid (70 mg); MS 433.88 (MH+).

### Example 4

### 5-Methyl-5-[1-({4-[(2-methylquinolin-4-yl)methoxy]phenyl}sulphonyl)ethyl]imidazolidine-2,4-dione

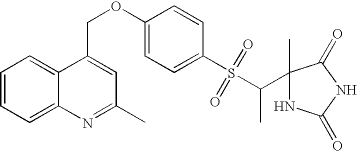

An analogous method to that described in example 1 was used except the starting material was 5-methyl-5-[1-({4-[(2-methylquinolin-4-yl)methoxy]phenyl}thio)ethyl]imidazolidine-2,4-dione (124 mg). The crude product was chromatographed on a 10 g silica bond elute using a 0-10% EtOH/DCM gradient as eluent to give 5-methyl-5-[1-({4-[(2-methylquinolin-4-yl)methoxy]phenyl}sulphonyl)ethyl]imidazolidine-2,4-dione as a white solid (64 mg); NMR δ 1.1 (d, 3H), 1.5 (s, 3H), 2.7 (s, 3H), 3.7 (m, 1H), 5.8 (s, 2H), 7.4 (d, 2H), 7.6 (d, 2H), 7.7 (d, 2H), 7.8 (d, 2H), 7.95 (d, 1H), 8.1 (d, 1H), 10.8 (s, 1H); MS 454.2 (MH+).

The starting material 5-methyl-5-[1-({4-[(2-methylquinolin-4-yl)methoxy]phenyl}thio)ethyl]imidazolidine-2,4-dione was prepared as follows:
i) To 4-(2-methylquinolin-4-ylmethoxy)thiophenol disulphide (example 1 step iv)) (358 mg) stirred at RT under argon, was added water (1 drop) and tri-n-butylphosphine (0.18 ml). Stirring was continued overnight and 3-bromo-2-butanone (0.188 ml), potassium carbonate (700 mg), and tetrabutylammonium iodide (10 g) were added. After 2 h excess inorganic material was filtered off, the filtrate concentrated in vacuo and purified on a 10 g silica bond elute using 0-1% EtOH/DCM gradient as eluent over 45 min to give 3-({4-[(2-methylquinolin-4-yl)methoxy]phenyl}thio)butan-2-one as a colourless oil (350 mg); MS 351.98(ES+).
ii) 3-({4-[(2-Methylquinolin-4-yl)methoxy]phenyl}thio)butan-2-one (350 mg) was treated using an analogous method to that used in example 1 step vii). The crude product was chromatographed on a 20 g silica bond elute using a 0-6% EtOH/DCM gradient over 50 min as eluent to give 5-methyl-5-[1-({4-[(2-methylquinolin-4-yl)methoxy]phenyl}thio)ethyl]imidazolidine-2,4-dione as a white solid (180 mg); MS 422.2 (MH+)

### Example 5

### 5-[2-(4-Fluorophenyl)-1-({4-[(2-methylquinolin-4-yl)methoxy]phenyl}sulphonyl)ethyl]-5-methylimidazolidine-2,4-dione

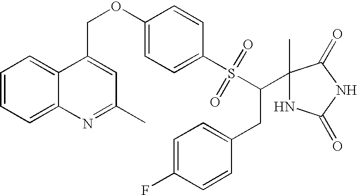

Single Diastereoisomer

An analogous method to the described in example 1 was used except that the starting material was 5-[2-(4-fluorophenyl)-1-({4-[(2-methylquinolin-4-yl)methoxy]phenyl}thio)ethyl]-5-methylimidazolidine-2,4-dione (90 mg). The crude product was chromatographed on a 10 g silica bond elute using a 0-15% EtOH(DCM gradient over 45 min as eluent to give 5-[2-(4-fluorophenyl)-1-({4-[(2-methylquinolin-4-yl)methoxy]phenyl}sulphonyl)ethyl]-5-methylimidazolidine-2,4-dione as a white solid (29 mg); MS 548.2 (MH+).

The starting material 5-[2-(4-fluorophenyl)-1-({4-[(2-methylquinolin-4-yl)methoxy]phenyl}thio)ethyl]-5-methylimidazolidine-2,4-dione was prepared by an analogous method to that described in example 4 steps i) and ii) except that 3-bromo-2-butanone in step i) was replaced with 3-chloro-4-(4-fluorophenyl)-2-butanone to yield 4-(4-fluorophenyl)-3-({4-[(2-methylquinolin-4-yl)methoxy]phenyl}thio)butan-2-one as a colourless oil (470 mg); MS 445.95 (MH+) and then 5-[2-(4-fluorophenyl)-1-({4-[(2-methylquinolin-4-yl)methoxy]phenyl}thio)ethyl]-5-methylimidazolidine-2,4-dione as a white solid (90 mg); MS 516.2 (MH+).

### Example 6

### 5-[({4-[(2-Methylquinolin-4-yl)methoxy]phenyl}sulphonyl)methyl]-5-(3-pyrimidin-2-ylpropyl)imidazolidine-2,4-dione

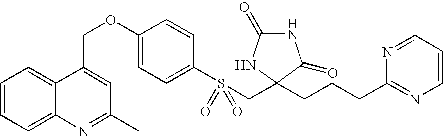

To 1-({4-[(2-methylquinolin-4-yl)methoxy]phenyl}sulphonyl)-5-pyrimidin-2-ylpentan-2-one (65 mg) dissolved in EtOH (3 ml) and water (3 ml) was added ammonium carbonate (318 mg) and potassium cyanide (18 mg). The mixture was stirred at 70° C. for 6 d. The solution was cooled to RT, partitioned between saturated brine (20 ml) and EtOAc (2×25 ml), and the combined organic extracts were concentrated in vacuo and purified on a 10 g silica bond elute using a 0-10% EtOH/DCM gradient over 50 min as eluent to give 5-[({4-[(2-methylquinolin-4-yl)methoxy]phenyl}sulphonyl)methyl]-5-(3-pyrimidin-2-ylpropyl)imidazolidine-2,4-dione as a white solid (27 mg); MS 545.96 (MH+).

The starting material 1-({4-[(2-methylquinolin-4-yl)methoxy]phenyl}sulphonyl)-5-pyrimidin-2-ylpentan-2-one was prepared as follows:
i) To 4-(2-methylquinolin-4-ylmethoxy)thiophenol disulphide (example 1 step iv)) (4 g), suspended in acetonitrile (100 ml) and stirred at RT under argon was added water (15 drops) and tri-n-butylphosphine (1.87 ml). Stirring was continued overnight and iodomethane (1.07 ml) and potassium carbonate (7.88 g) were added. After 1 h excess inorganic residues were filtered off and the filtrate concentrated in vacuo. The residue was purified on a 100 g silica bond elute using a 20-80% EtOAc/isohexane gradient over 45 min as eluent to give 4-(2-methylquinolin-4-ylmethoxy)phenylthiomethane as a light brown oil (2.91 g); NMR δ 2.45 (s, 3H), 2.65 (s, 3H), 5.6 (s, 2H), 7.05 (d, 2H), 7.25 (d, 2H), 7.5 (m, 2H), 7.7 (t, 1H), 7.95 (d, 1H), 8.1 (d, 1H); MS 295.99 (MH+).
ii) 4-(2-methylquinolin-4-ylmethoxy)phenylthiomethane (2.9 g) was stirred at RT in MeOH (90 ml) and a suspension of potassium peroxymonosulphate (9.05 g) in water (60 ml) was added. After 1 h the resulting white precipitate was filtered off and partitioned between saturated aqueous potassium carbonate (250 ml) and DCM (2×300 ml). The combined organic extracts were concentrated in vacuo and purified on a 100 g silica bond elute using a 40-80% EtOAc/isohexane gradient over 50 min as eluent to give 4-(2-methylquinolin-4-ylmethoxy)phenylsulphonylmethane as a white solid (2.38 g); NMR δ 2.65 (s, 3H), 3.2 (s, 3H), 5.75 (s, 2H), 7.4 (d, 2H), 7.6 (m, 2H), 7.7 (t, 1H), 7.9 (d, 2H), 8.0 (d, 1H), 8.1 (d, 1H); MS 328.3 (MH+).
iii) 4-(2-Methylquinolin-4-ylmethoxy)phenylsulphonylmethane (300 mg) was suspended in THF (6 ml) and stirred at −10° C. under argon. HMDS (1.0M in THF, 0.96 ml) was added, followed after 10 min by a solution of ethyl 4-(2-pyrimidinyl)butyrate (178 mg) in THF (2 ml). After 1 h the reaction was quenched with saturated aqueous ammonium chloride (10 ml) and partitioned with EtOAc (2×15 ml). The combined organic extracts were dried (sodium sulphate) and concentrated in vacuo. The residue was purified on a 20 g silica bond elute using a 0-5% EtOH/DCM gradient over 50 min as eluent to give 1-({4-[(2-methylquinolin-4-yl)methoxy]phenyl}sulphonyl)-5-pyrimidin-2-ylpentan-2-one as a pale yellow oil (70 mg); NMR δ 1.9 (m, 2H), 2.65 (m, 5H), 2.8 (t, 2H), 4.6 (s, 2), 5.75 (s, 2H), 7.35 (m, 2H), 7.5 (m, 2H), 7.75 (m, 2H), 7.8 (d, 2H), 8.0 (d, 1H), 8.1 (d, 1H), 8.7 (d, 2H); MS 476.2 (MH+).

### Example 7

### 5-Isopropyl-5-[({4-[(2-methylquinolin-4-yl)methoxy]phenyl}sulphonyl)methyl]imidazolidine-2,4-dione

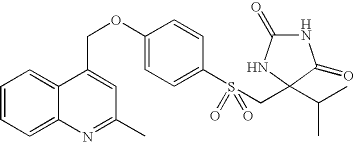

5-Isopropyl-5-[({4-[(2-methylquinolin-4-yl)methoxy]phenyl}sulphonyl)methyl]imidazolidine-2,4-dione was prepared using an analogous method to that described in example 6 except that in step iii) ethyl 4-(2-pyrimidinyl)butyrate was replaced with methyl isobutyrate to yield the product as a white solid (37 mg); MS 468.2 (MH+).

### Example 8

### 5-(Methoxymethyl)-5-[({4-[(2-methylquinolin-4-yl)methoxy]phenyl}sulphonyl)methyl]imidazolidine-2,4-dione

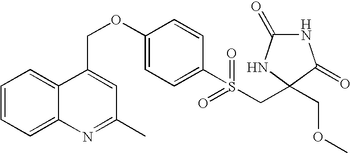 5-(Methoxymethyl)-5-[({4-[(2-methylquinolin-4-yl)methoxy]phenyl}sulphonyl)methyl]imidazolidine-2,4-dione was prepared using an analogous method to that described in example 6 except that in step iii) ethyl 4-(2-pyrimidinyl)butyrate was replaced with methyl methoxyacetate to yield the product as a white solid (37 mg); MS 470.2 (MH+).

### Example 9

### 5-(2-Methoxyethyl)-5-[({4-[(2-methylquinolin-4-yl)methoxy]phenyl}sulphonyl)methyl]imidazolidine-2,4-dione

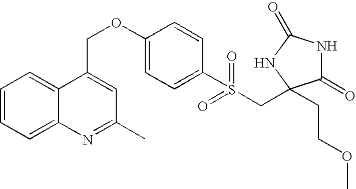

5-(2-Methoxyethyl)-5-[({4-[(2-methylquinolin-4-yl)methoxy]phenyl}sulphonyl)methyl]imidazolidine-2,4-dione was prepared using an analogous method to that described in example 6 except that in step iii) ethyl 4-(2-pyrimidinyl)butyrate was replaced with methyl 3-methoxypropionate to yield the product as a light brown solid (9 mg); MS 483.99 (MH+).

### Example 10

### tert-Butyl ({4-[({4-[(2-methylquinolin-4-yl)methoxy]phenyl}sulphonyl)methyl]-2,5-dioxoimidazolidin-4-yl}methyl)carbamate

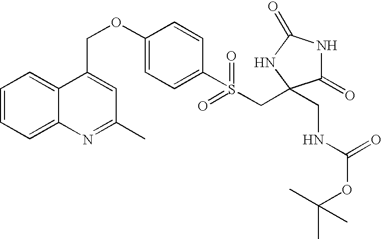 tert-Butyl ({4-[({4-[(2-methylquinolin-4-yl)methoxy]phenyl}sulphonyl)methyl]-2,5-dioxoimidazolidin-4-yl}methyl)carbamate was prepared using an analogous method to that described in example 6 except that ethyl 4-(2-pyrimidinyl)butyrate was replaced with methyl N-(tert-butoxycarbonyl)glycinate to yield the product as a white solid (13 mg); MS 555.2 (MH+).

### Example 11

### tert-Butyl 4-{4-[({4-[(2-methylquinolin-4-yl)methoxy]phenyl}sulphonyl)methyl]-2,5 dioxoimidazolidin-4-yl}piperidin-1-ylcarboxylate

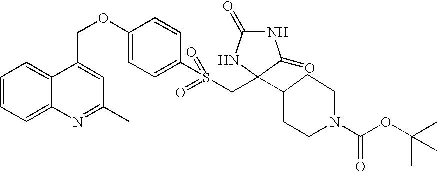 tert-Butyl 4-{4-[({4-[(2-methylquinolin-4-yl)methoxy]phenyl}sulphonyl)methyl]-2,5-dioxoimidazolidin-4-yl}piperidin-1-ylcarboxylate was prepared using an analogous method to that described in example 6 except that in step iii) ethyl 4(2-pyrimidinyl)butyrate was replaced with ethyl (N-[tert-butoxycarbonyl]piperidin-4-yl)carboxylate to yield the product as a white solid (9 mg); MS 608.99 (MH+).

### Example 12

### 5-(1-Acetylpiperidin-4-yl)-5-[({4-[(2-methylquinolin-4-yl)methoxy]phenyl}sulphonyl)methyl]imidazolidine-2,4-dione

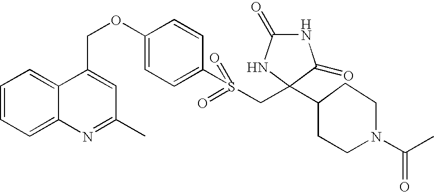

1-(1-Acetylpiperidin-4-yl)-2-({4-[(2-methylquinolin-4-yl)methoxy]phenyl}sulphonyl)ethanone (65 mg) was treated using an analogous method to that described in example 6 to yield 5-(1-acetylpiperidin-4-yl)-5-[({4-[(2-methylquinolin-4-yl)methoxy]phenyl}sulphonyl)methyl]imidazolidine-2,4-dione as a white solid (3 mg); MS 550.90 (MH+).

The starting material 1-(1-acetylpiperidin-4-yl)-2-({4-[(2-methylquinolin-4-yl)methoxy]phenyl}sulphonyl)ethanone was prepared as follows:
i) To tert-butyl 4-[({4-[(2-methylquinolin-4-yl)methoxy]phenyl}sulphonyl)acetyl]piperidine-1-carboxylate (example 11 step iii)) (220 mg) in MeOH (10 ml) at RT was added hydrogen chloride (4M in 1,4-dioxan, 20 ml). After 2 h the solution was concentrated in vacuo and azeotroped once with toluene. This gave 2-({4-[(2-methylquinolin-4-yl)methoxy]phenyl}sulphonyl)-1-piperidin-4-ylethanone as a white solid (210 mg); NMR δ 1.3 (m, 1H), 1.6 (m, 2H), 2.0 (m, 2H), 2.8 (m, 3H), 3.0 (s, 3H), 4.8 (s, 2H), 6.0 (s, 2H), 7.45 (d, 2H), 7.9 (m, 3H), 8.1 (m, 2H), 8.4 (d, 2H), 8.9 (m, 1H), 9.1 (m, 1H); MS 439.18 (MH+).
ii) To a suspension of 2-({4-[(2-methylquinolin-4-yl)methoxy]phenyl}sulphonyl)-1-piperidin-4-ylethanone (70 mg) in acetonitrile (5 ml) at RT was added triethylamine (0.096 ml) followed by acetyl chloride (0.01 ml). After 1 h the mixture was diluted with EtOAc (20 ml) and partitioned with brine (10 ml). The organic portion was concentrated in vacuo and purified on a 10 g silica bond elute using a 0-5% EtOH/DCM gradient over 50 min as eluent to give 1-(1-acetylpiperidin-4-yl)-2-({4-[(2-methylquinolin-4-yl)methoxy]phenyl}sulphonyl)ethanone as a white solid (70 mg); NMR δ 1.1 (m, 3H), 1.8 (m, 2H), 1.95 (s, 3H), 2.65 (s, 3H), 2.75 (m, 1H), 3.0 (m, 1H), 3.8 (m, 1H), 4.25 (m, 1H), 4.8 (s, 2H), 5.75 (s, 2H), 7.35 (d, 2H), 7.6 (m, 2H), 7.7 (t, 1H), 7.85 (d, 2H), 7.95 (d, 1H), 8.1 (d, 1H); MS 481.2 (MH+).

### Example 13

### 5-(Aminomethyl)-5-[({4-[(2-methylquinolin-4-yl)methoxy]phenyl}sulphonyl)methyl]imidazolidine-2,4-dione

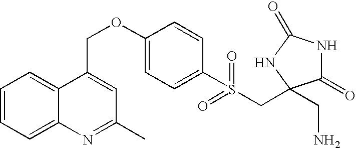

To tert-butyl({4-[({4-[(2-methylquinolin-4-yl)methoxy]phenyl}sulphonyl)methyl]-2,5-dioxoimidazolidin-4-yl}methyl)carbamate (20 mg) (example 10) dissolved in MeOH (5 ml) was added hydrogen chloride (4M in 1,4-dioxan, 1.5 ml). The solution was stirred at RT overnight and partitioned between saturated aqueous potassium carbonate (10 ml) and DCM (2×15 ml). The combined organic extracts were dried (sodium sulphate) and concentrated in vacuo to give 5-(aminomethyl)-5-[({4-[(2-methylquinolin-4-yl)methoxy]phenyl}sulphonyl)methyl]imidazolidine-2,4-dione as a white solid (10 mg); MS 454.92 (MH+).

### Example 14

### 5-Isobutyl-5-[({4-[(2-methylquinolin-4-yl)methoxy]phenyl}sulphonyl)methyl]imidazolidine-2,4-dione

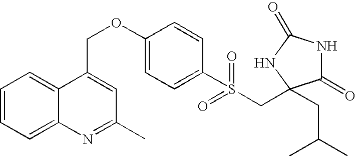

5-Isobutyl-5-[({4-[(2-methylquinolin-4-yl)methoxy]phenyl}sulphonyl)methyl]imidazolidine-2,4-dione was prepared by an analogous method to that described in example 6 except that in step iii) ethyl 4-(2-pyrimidyl)butyrate was replaced with ethyl isovalerate to yield the product as a white solid (23 mg); MS 481.96 (MH+).

### Example 15

### 5-Cyclopropyl-5-[({4-[(2-methylquinolin-4-yl)methoxy]phenyl}sulphonyl)methyl]imidazolidine-2,4-dione

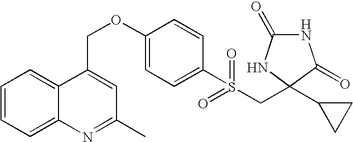

5-Cyclopropyl-5-[({4-[(2-methylquinolin-4-yl)methoxy]phenyl}sulphonyl)methyl]imidazolidine-2,4-dione was prepared by an analogous method to that described in example 6 except that in step iii) ethyl 4-(2-pyrimidyl)butyrate was replaced with ethyl cyclopropanecarboxylate to yield the product as a white solid (10 mg); MS 465.85 (MH+).

### Example 16

### 5-[({4-[(3,5-Dimethoxybenzyl)oxy]phenyl}sulphonyl)methyl]-5-methylimidazolidine-2,4-dione

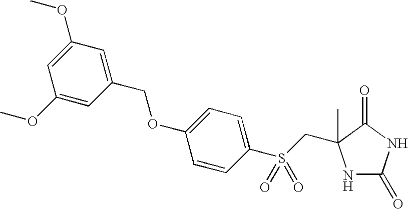 Sodium hydride (48 mg of 60% dispersion) was added to a solution of 3,5-dimethoxybenzyl alcohol (168 mg) in dimethylacetamide (10 ml) and the mixture stirred at ambient temperature for 20 min. 5-{[(4-fluorophenyl)sulphonyl]methyl}-5-methylimidazolidine-2,4-dione (286 mg) was added and the reaction mixture was heated for 4 h at 70° C. After cooling, the reaction was poured into water (50 ml) and the solution acidified to pH1 using 36% hydrochloric acid.

The resulting precipitate was filtered and washed with water. The product was then treated with ether and dried in vacuo to afford 5-[({4[(3,5-dimethoxybenzyl)oxy]phenyl}sulphonyl)methyl]-5-methylimidazolidine-2,4-dione. NMR (DMSO-d6) 1.25 (s, 3H), 3.7 (m, 2H), 3.8 (s, 6), 5.15 (s, 2H), 6.5 (m, 1H), 6.6 (m, 2H), 7.25 (m, 2H), 7.8 (m, 3H), 10.7 (s, 1H). MS 435 (MH+).

The starting material 5-{[(4-fluorophenyl)sulphonyl]methyl}-5-methylimidazolidine-2,4-dione was prepared as follows:
i) A solution of 1-[(4-fluorophenyl)thio]acetone (1.4 g) [J. Het. Chem. 10 (1973) 127] in 50% aqueous ethanol (40 ml) was stirred at ambient temperature and ammonium carbonate (4.5 g) and potassium cyanide (1.0 g) were added. The mixture was heated at 55° C. for 3 h, cooled and evaporated to dryness. The residual solid was treated with water, washed with water then with ether and dried to afford 5-{[(4-fluorophenyl)thio]methyl}-5-methylimidazolidine-2,4-dione; NMR (DMSO-d6) δ 1.3 (s, 3H), 3.2 (s, 2H), 7.1 (m, 2H), 7.45 (m, 2H), 7.9 (s, 1H), 10.7 (br, 1H). MS 253 (MH−).
ii) A slurry of potassium peroxymonosulphate (30.5 g) in water (30 ml) was added to a stirred, ice-cooled solution of 5-{[(4-fluorophenyl)thio]methyl}-5-merthylimidazolidine-2,4-dione (4.2 g) in MeOH (150 ml) The temperature was adjusted to ambient and the mixture was stirred for 18 h. The solid was filtered and treated with 1M hydrochloric acid then washed with water and dried to afford 5-{[(4-fluorophenyl)sulphonyl]methyl}-5-methylimidazolidine-2,4-dione; NMR (DMSO-d6) δ 1.3 (s, 3H), 3.8 (m, 2H), 7.45 (m, 2H), 7.8 (s, 1H), 7.9 (m, 2H), 10.75 (s, 1H); MS 285 (MH−).

### Example 17

### 5-Methyl-5-({[4-(1-naphthylmethoxy)phenyl]sulphonyl}methyl)imidazoldine-2,4-dione

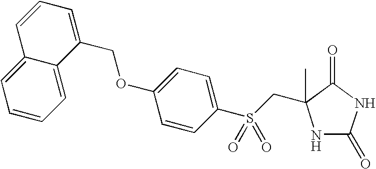

5-Methyl-5-({[4-(1-naphthylmethoxy)phenyl]sulphonyl}methyl)imidazolidine-2,4-dione was prepared using an analogous method to that described in example 16 except that 3,5-dimethoxybenzyl alcohol was replaced with 1-naphthylmethanol (241 mg) to afford 5-methyl-5-({[4-(1-naphthylmethoxy)phenyl]sulphonyl}methyl)imidazolidine-2,4-dione; NMR (DMSO-d6) δ 1.3 (s, 3H), 3.7 (m, 2H), 5.7 (s, 2H), 7.3 (m, 2H), 7.5 (m, 3H), 7.7 (m, 4H), 7.9 (m, 2H), 8.1 (m, 1H), 10.7 (s, 1H); MS 423 (MH−).

### Example 18

### 6-({4-[(3,5-Dimethoxybenzyl)oxy]phenyl}sulphonyl)-1,3-diazaspiro[4.4]nonane-2,4-dione

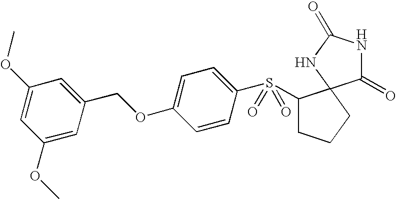

6-({4-[(3,5-Dimethoxybenzyl)oxy]phenyl}sulphonyl)-1,3-diazaspiro[4.4]nonane-2,4-dione was prepared by an analogous method to that described in example 16 except 6-[(4-fluorophenyl)sulphonyl]-1,3-diazaspiro[4.4]nonane-2,4-dione (311 mg) and 3,5-dimethoxybenzyl alcohol were used; NMR DMSO-d_{6 1.8} (m, 2H), 2.0 (m, 4H), 3.75 (s, 6H), 3.8 (m, 1H), 5.3 (s, 2H), 6.45 (m, 1H), 6.6 (m, 2H), 7.2 (m, 2H), 7.7 (m, 2H), 8.35 (s, 1H), 10.65 (br, 1H); MS 459 (MH−).

The starting material 6-[(4-fluorophenyl)sulphonyl]-1,3-diazaspiro[4.4]nonane-2,4-dione was prepared as follows:
i) 2-Chlorocyclopentanone (2.84 g) was added to an ice-cold mixture of 4-fluorothiophenol (2.56 g) and an aqueous 1M solution of sodium hydroxide (20 ml) in MeOH (100 ml) with stirring. The resulting solution was allowed to warm to ambient temperature and stirred for 90 min. After removal of the solvent, the residue was partitioned between EtOAc and saturated aqueous sodium carbonate. The solvent phase was washed with water and then dried (MgSO₄) and evaporated to dryness. 2-[(4-fluorophenyl)thio]cyclopentanone was used without further purification.
ii) 2-[(4-Fluorophenyl)thio]cyclopentanone (3.2 g) was treated using an analogous method to that described in example 16 step i) to afford 6-[(4-fluorophenyl)thio]-1,3-diazaspiro[4.4]nonane-2,4-dione; NMR (DMSO-d₆) δ 1.8 (m, 4H), 2.2 (m, 2H), 3.6 (m, 1H), 7.2 (m, 2H), 7.4 (m, 2H), 8.4 (s, 1H), 10.59 (br, 1H); MS 279 (MH−).
iii) 6-[(4-Fluorophenyl)thio]-1,3-diazaspiro[4.4]nonane-2,4-dione (2.8 g) was treated using an analogous process to that described in example 16 step ii) to yield 6-[(4-fluorophenyl)sulphonyl]-1,3-diazaspiro[4.4]nonane-2,4-dione; NMR (DMSO-d₆) δ 1.7 (m, 2H), 2.0 (m, 4H), 3.9 (m, 1H), 7.45 (m, 2H), 7.8 (m, 2H), 8.4 (s, 1H), 10.68 (br, 1H); MS 311 (MH−).

### Example 19

### 6-{[4-(1-Naphthylmethoxy)phenyl]sulphonyl}-1,3-diazaspiro[4.4]nonane-2,4-dione

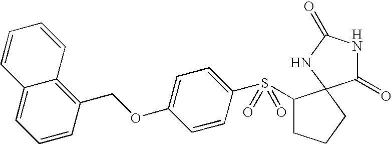

6-{[4-(1-Naphthylmethoxy)phenyl]sulphonyl}-1,3-diazaspiro[4.4]nonane-2,4-dione was prepared by an analogous method to that described in example 18 except that 3,5-dimethoxybenzyl alcohol was replaced with 1-naphthylmethanol; NMR DMSO-d6 1.8 (m, 2H), 2.0 (m, 4H), 3.8 (m, 1H), 5.7 (s, 2H), 7.3 (m, 2H), 7.6 (m, 3H), 7.7 (m, 3H), 7.95 (m, 2H), 8.1 (m, 1H), 8.4 (s, 1H), 10.65 (br, 1H); MS 449 (MH−).

### Example 20

### 5-Methyl-5-({[4-(quinolin-4-ylmethoxy)phenyl]sulphonyl}methyl)imidazolidine-2,4-dione

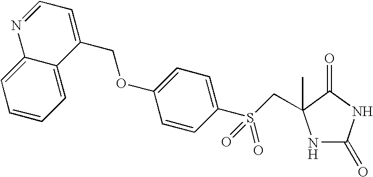

A slurry of potassium peroxymonosulphate (0.57 g) in water (5 ml) was added to a stirred solution of 5-methyl-5-({[4-(quinolin-4-ylmethoxy)phenyl]thio}methyl)imidazolidine-2,4-dione (120 mg) in MeOH (20 ml) and the mixture stirred at ambient temperature for 3 h. After filtration from the inorganic material, the filtrate was evaporated The crude product was purified initially with an SCX column (eluant gradient MeOH to 2M ammonia in MeOH) and then with a silica column (eluant gradient DCM to 10% MeOH/DCM) to give the title compound (25 mg);

NMR DMSOd6 1.3 (s, 3H), 3.7 (m, 2H), 5.75 (m, 2H), 7.4 (m, 2H), 7.7 (m, 2H), 7.8 (m, 4H), 8.2 (m, 1H), 8.6 (m, 1H), 9.0 (m, 1H), 10.7 (s, 1H); MS 424 (MH−).

The starting material 5-methyl-5-({[4-(quinolin-4-ylmethoxy)phenyl]thio}methyl)imidazolidine-2,4-dione was prepared as follows:
i) A solution of 4-hydroxythiophenol (1.26 g) in MeOH (50 ml) was cooled to 5° C. and 1M aqueous sodium hydroxide (10 ml) was added. Chloroacetone (0.96 ml) was added dropwise with stirring over 5 min and the mixture was stirred for a further 30 min. After evaporation, the residue was partitioned between 2M hydrochloric acid and EtOAc. The solvent layer was washed with water, dried with magnesium sulphate and evaporated to an oil. The crude product was purified by silica chromatography (eluant gradient DCM to 5% MeOH/DCM) to give 1-[(4-hydroxyphenyl)thio]acetone (1.0 g); NMR (CDCl₃) 2.3 (s, 3H), 3.5 (s, 2H), 6.75 (m, 2H), 7.3 (m, 2H); MS 181 (MH−).
ii) A mixture of 4-chloromethylquinoline hydrochloride (0.5 g), 1-[(4-hydroxyphenyl)thio]acetone (0.5 g), potassium iodide (20 mg) and potassium carbonate (0.95 g) was stirred at 60° C. for 4 h. The reaction was cooled and the inorganic solid filtered off and the filtrate was evaporated to an oil. Purification was achieved by an SCX column (eluant gradient MeOH to 2M solution of ammonia in MeOH) to yield 1-{[4-(quinolin-4-ylmethoxy)phenyl]thio}acetone as an oil (0.44 g) which was used without further purification; MS 322 (MH−).
iii) A solution of 1-{[4-(quinolin-4-ylmethoxy)phenyl]thio}acetone (0.44 g) in 50% aqueous EtOH (25 ml) was stirred at 55° C. and potassium cyanide (0.18 g) and ammonium carbonate (0.82 g) were added and the mixture stirred for 3 h. The reaction was evaporated to half volume, filtered and the filtrate evaporated to dryness. The crude product was purified by silica chromatography (eluant gradient DCM to 10% MeOH/DCM) to give 5-methyl-5-({[4-(quinolin-4-ylmethoxy)phenyl]thio}methyl)imidazolidine-2,4-dione (0.12 g); NMR DMSOd6 1.3 (s, 3H), 3.2 (m, 2H), 5.7 (m, 2H), 7.4 (m, 2H), 7.7 (m, 2H), 7.8 (m, 4H), 8.2 (m, 1H), 8.6 (m, 1H), 9.0 (m, 1H), 10.7 (s, 1H); MS 392 (MH−).

1. A compound of formula (IA) or a pharmaceutically acceptable salt thereof: 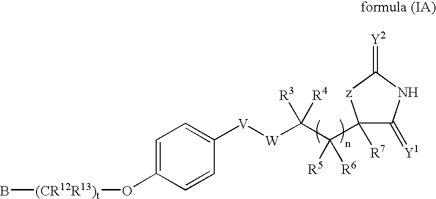
wherein:
Y¹ and Y² are both O;
z is NR⁸, O or S;
n is 0 or 1;
W is NR¹, CR¹R² or a bond;
V is NR¹⁵SO₂;
t is 0 or 1;
B is a group selected from aryl, heteroaryl and heterocyclyl where each group is optionally substituted by one or more groups independently selected from nitro, trifluoromethyl, trifluoromethoxy, halo, cyano, C₁₋₄alkyl [symbol] optionally substituted by R⁹ or C₁₋₄alkoxy or one or more halo [symbol] , C₂₋₄alkenyl [symbol] optionally substituted by halo or R⁹ [symbol] , C₂₋₄alkynyl [symbol] optionally substituted by halo or R⁹ [symbol] , C₃₋₆cycloalkyl [symbol] optionally substituted by R⁹ or one or more halo [symbol] , C₅₋₆cycloalkenyl [symbol] optionally substituted by halo or R⁹ [symbol] , aryl [symbol] optionally substituted by halo or C₁₋₄alkyl [symbol] , heteroaryl [symbol] optionally substituted by halo or C₁₋₄alkyl [symbol] , heterocyclyl [symbol] optionally substituted by C₁₋₄alkyl [symbol] , —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl and heterocyclyl which group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl or C₁₋₄alkoxy;
R¹ and R² are independently hydrogen or a group selected from C₁₋₆alkyl, C₂₋₆alkenyl, C₂₋₆alkynyl, C₃₋₆cycloalkyl and C₅₋₆cycloalkenyl which the group may be optionally substituted by halo, cyano, nitro, hydroxy or C₁₋₄alkoxy;
R³, R⁴, R⁵ and R⁶ are independently hydrogen or a group selected from C₁₋₆alkyl, C₂₋₆alkenyl, C₂₋₆alkynyl, C₃₋₆cycloalkyl, C₅₋₆cycloalkenyl, aryl, heteroaryl and heterocyclyl which group is optionally substituted by one or more substituents independently selected from halo, nitro, cyano, trifluoromethyl, trifluoromethoxy, C₁₋₄alkyl, C₂₋₄alkenyl, C₂₋₄alkynyl, C₃₋₆cycloalkyl [symbol] optionally substituted by one or more R¹⁷ [symbol] , aryl [symbol] optionally substituted by one or more R¹⁷ [symbol] , heteroaryl [symbol] optionally substituted by one or more R¹⁷ [symbol] , heterocyclyl, —OR¹⁸, —SR¹⁹, —SOR¹⁹, —SO₂R¹⁹, —COR¹⁹, —CO₂R¹⁸, —CONR¹⁸R²⁰, —NR¹⁶COR¹⁸, —SO₂NR¹⁸R²⁰ and —NR¹⁶SO₂R¹⁹;
or R¹ and R³ together with the nitrogen or carbon atoms and carbon atom to which they are respectively attached form a saturated 3- to 7-membered ring optionally containing 1 or 2 heteroatoms groups selected from NH, O, S, SO and SO₂ where the ring is optionally substituted on carbon by C₁₋₄alkyl, C₁₋₃alkoxy or fluoro and/or on nitrogen by —COC₁₋₃alkyl, —SO₂C₁₋₃alkyl or C₁₋₄alkyl;
or R³ and R⁴ together with the carbon atom to which they are attached form a saturated 3- to 7-membered ring optionally containing a heteroatom group selected from NH, O, S, SO and SO₂ where the ring is optionally substituted on carbon by C₁₋₄alkyl, C₁₋₃alkoxy or fluoro and/or on nitrogen by —COC₁₋₃alkyl, —SO₂C₁₋₃alkyl and/or C₁₋₄alkyl;
or R³ and R⁵ together with the carbon atoms to which they are attached form a saturated 3- to 7-membered ring optionally containing a heteroatom group selected from NH, O, S, SO and SO₂ where the ring is optionally substituted on carbon by C₁₋₄alkyl, C₁₋₃alkoxy or fluoro and/or on nitrogen by —COC₁₋₃alkyl, —SO₂C₁₋₃alkyl or C₁₋₄alkyl;
or R⁵ and R⁶ together with the carbon atom to which they are attached form a saturated 3- to 7-membered ring optionally containing a heteroatom group selected from NH, O, S, SO and SO₂ where the ring is optionally substituted on carbon by C₁₋₄alkyl, C₁₋₃alkoxy or fluoro and/or on nitrogen by —COC₁₋₃alkyl, —SO₂C₁₋₃alkyl or C₁₋₄alkyl;
R⁷ is hydrogen or a group selected from C₁₋₆alkyl, C₂₋₆alkenyl, C₂₋₆alkynyl, heteroalkyl, C₃₋₇cycloalkyl, aryl, heteroaryl and heterocyclyl where the group is optionally substituted by halo, C₁₋₄alkyl, C₁₋₄alkoxy, C₃₋₇cycloalkyl, heterocyclyl, aryl, heteroaryl or heteroalkyl; and
wherein the group from which R⁷ may be selected is optionally substituted on the group and/or on its optional substituent by one or more substituents independently selected from halo, cyano, C₁₋₄alkyl, nitro, haloC₁₋₄alkyl, heteroalkyl, aryl, heteroaryl, hydroxyC₁₋₄alkyl, C₃₋₇cycloalkyl, heterocyclyl, C₁₋₄alkoxyC₁₋₄alkyl, haloC₁₋₄alkoxyC₁₋₄alkyl, —COC₁₋₄alkyl, —OR²¹, —NR²¹R²², —CO₂R²¹, —SR²⁵, —SOR²⁵, —SO₂R²⁵, —NR²¹COR²², —NR²¹CO₂R²², —CONR²¹R²² and —NHCONR²¹R²²;
or R³ and R⁷ together with the carbon atoms to which they are each attached and (CR⁵R⁶)ₙ form a saturated 5- to 7-membered ring optionally containing a heteroatom group selected from NH, O, S, SO and SO₂ where the ring is optionally substituted on carbon by C₁₋₄alkyl, C₁₋₃alkoxy or fluoro and/or on nitrogen by —COC₁₋₃alkyl, —SO₂C₁₋₃alkyl or C₁₋₄alkyl;
R⁸ is selected from hydrogen or methyl;
R⁹ and R¹⁰ are independently hydrogen, C₁₋₆alkyl or C₃₋₆cycloalkyl;
or R⁹ and R¹⁰ together with the nitrogen to which they are attached form a heterocyclic 4- to 7-membered ring;
R¹¹ is C₁₋₆alkyl or C₃₋₆cycloalkyl;
R¹² and R¹³ are independently selected from hydrogen, C₁₋₆alkyl and C₃₋₆cycloalkyl;
R¹⁵ is hydrogen or C₁₋₃alkyl;
R¹⁶ is hydrogen or C₁₋₆alkyl;
R¹⁷ is selected from halo, C₁₋₆alkyl, C₃₋₆cycloalkyl and C₁₋₆alkoxy;
R¹⁸ is hydrogen or a group selected from C₁₋₆alkyl, C₃₋₆cycloalkyl, C₅₋₆cycloalkenyl, saturated heterocyclyl, aryl, heteroaryl, arylC₁₋₄alkyl and heteroarylC₁₋₄alkyl where the group is optionally substituted by one or more halo;
R¹⁹ and R²⁵ are independently a group selected from C₁₋₆alkyl, C₃₋₆cycloalkyl, C₅₋₆cycloalkenyl, saturated heterocyclyl, aryl, heteroaryl, arylC₁₋₄alkyl and heteroarylC₁₋₄alkyl where the group is optionally substituted by one or more halo;
R²⁰ is hydrogen, C₁₋₆alkyl or C₃₋₆cycloalkyl;
or R¹⁸ and R²⁰ together with the nitrogen atom to which they are attached form a heterocyclic 4- to 7-membered ring;
R²¹ and R²² are independently hydrogen, C₁₋₄alkyl, haloC₁₋₄alkyl, aryl and arylC₁₋₄alkyl; provided a compound of formula (IA) is not 1-(4-methyl-2,5-dioxoimidazolidin-4-yl)-N-[4-(4-chlorophenoxy)phenyl]methanesulphonamide.

2. A compound of formula (IB) or a pharmaceutically acceptable salt thereof: 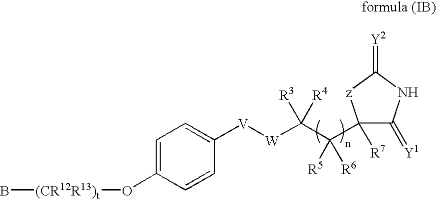
wherein:
Y¹ and Y² are independently O;
z is NR⁸, O or S;
n is 0 or 1;
W is NR¹;
V is SO₂ or CO;
t is 0 or 1;
B is a group selected from aryl, heteroaryl and heterocyclyl where each group is optionally substituted by one or more groups independently selected from nitro, trifluoromethyl, trifluoromethoxy, halo, cyano, C₁₋₄alkyl [symbol] optionally substituted by R⁹ or C₁₋₄alkoxy or one or more halo [symbol] , C₂₋₄alkenyl [symbol] optionally substituted by halo or R⁹ [symbol] , C₂₋₄alkynyl [symbol] optionally substituted by halo or R⁹ [symbol] , C₃₋₆cycloalkyl [symbol] optionally substituted by R⁹ or one or more halo [symbol] , C₅₋₆cycloalkenyl [symbol] optionally substituted by halo or R⁹ [symbol] , aryl [symbol] optionally substituted by halo or C₁₋₄alkyl [symbol] , heteroaryl [symbol] optionally substituted by halo or C₁₋₄alkyl [symbol] , heterocyclyl [symbol] optionally substituted by C₁₋₄alkyl [symbol] , —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl and heterocyclyl which group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl or C₁₋₄alkoxy;
provided that when t is 0 such that B is directly attached to the oxygen atom shown in formula (IB) and B is monocyclic aryl, monocyclic heteroaryl or monocyclic heterocyclyl and n is 0 then the monocyclic group that is B is substituted on one of the atoms adjacent to the atom to which the oxygen is attached, by a group selected from those listed above in the definition of B which optionally substitute B;
R¹ and R³ together with the nitrogen and carbon atoms to which they are respectively attached form a saturated 3- to 7-membered ring optionally containing a further heteroatom group selected from NH, O, S, SO and SO₂ where the ring is optionally substituted on carbon by C₁₋₄alkyl, fluoro or C₁₋₄alkoxy and/or on nitrogen by —COC₁₋₃alkyl, —SO₂C₁₋₃alkyl or C₁₋₄alkyl;
R⁴, R⁵ and R⁶ are independently hydrogen or a group selected from C₁₋₆alkyl, C₂₋₆alkenyl, C₂₋₆alkynyl, C₃₋₆cycloalkyl, C₅₋₆cycloalkenyl, aryl, heteroaryl and heterocyclyl which group is optionally substituted by one or more substituents independently selected from halo, nitro, cyano, trifluoromethyl, trifluoromethoxy, C₁₋₄alkyl, C₂₋₄alkenyl, C₂₋₄alkynyl, C₃₋₆cycloalkyl [symbol] optionally substituted by one or more R¹⁷ [symbol] , aryl [symbol] optionally substituted by one or more R¹⁷ [symbol] , heteroaryl [symbol] optionally substituted by one or more R¹⁷ [symbol] , heterocyclyl, —OR¹⁸, —SR¹⁹, —SOR¹⁹, —SO₂R¹⁹, —COR¹⁹, —CO₂R¹⁸, —CONR¹⁸R²⁰, —NR¹⁶COR¹⁸, —SO₂NR¹⁸R²⁰ and —NR¹⁶SO₂R¹⁹;
or R⁵ and R⁶ together with the carbon atom to which they are attached form a saturated 3- to 7-membered ring optionally containing a heteroatom group selected from NH, O, S, SO and SO₂ where the ring is optionally substituted on carbon by C₁₋₄alkyl, fluoro or C₁₋₄alkoxy and/or on nitrogen by —COC₁₋₃alkyl, —SO₂C₁₋₃alkyl or C₁₋₄alkyl;
R⁷ is hydrogen or a group selected from C₁₋₆alkyl, C₂₋₆alkenyl, C₂₋₆alkynyl, heteroalkyl, C₃₋₇cycloalkyl, aryl, heteroaryl or heterocyclyl where the group is optionally substituted by halo, C₁₋₄alkyl, C₁₋₄alkoxy, C₃₋₇cycloalkyl, heterocyclyl, aryl, heteroaryl and heteroalkyl; and wherein the group from which R⁷ may be selected is optionally substituted on the group and/or on its optional substituent by one or more substituents independently selected from halo, cyano, C₁₋₄alkyl, nitro, haloC₁₋₄alkyl, heteroalkyl, aryl, heteroaryl, hydroxyC₁₋₄alkyl, C₃₋₇cycloalkyl, heterocyclyl, C₁₋₄alkoxyC₁₋₄alkyl, haloC₁₋₄alkoxyC₁₋₄alkyl, —COC₁₋₄alkyl, —OR²¹, —NR²¹R²², —CO₂R²¹, —SR²⁵, —SOR²⁵, —SO₂R²⁵, —NR²¹COR²², —NR²¹CO₂R²², —CONR²¹R²² and —NHCONR²¹R²²;
R⁸ is selected from hydrogen or methyl;
R⁹ and R¹⁰ are independently hydrogen, C₁₋₆alkyl or C₃₋₆cycloalkyl;
or R⁹ and R¹⁰ together with the nitrogen to which they are attached form a heterocyclic 4- to 7-membered ring;
R¹¹ is C₁₋₆alkyl or C₃₋₆cycloalkyl;
R¹² and R¹³ are independently selected from hydrogen, C₁₋₆alkyl and C₃₋₆cycloalkyl;
R¹⁶ is hydrogen or C₁₋₆alkyl;
R¹⁷ is selected from halo, C₁₋₆alkyl, C₃₋₆cycloalkyl and C₁₋₆alkoxy;
R¹⁸ is hydrogen or a group selected from C₁₋₆alkyl, C₃₋₆cycloalkyl, C₅₋₆cycloalkenyl, saturated heterocyclyl, aryl, heteroaryl, arylC₁₋₄alkyl and heteroarylC₁₋₄alkyl where the group is optionally substituted by one or more halo;
R¹⁹ and R²⁵ are independently a group selected from C₁₋₆alkyl, C₃₋₆cycloalkyl, C₅₋₆cycloalkenyl, saturated heterocyclyl, aryl, heteroaryl, arylC₁₋₄alkyl and heteroarylC₁₋₄alkyl where the group is optionally substituted by one or more halo;
R²⁰ is hydrogen, C₁₋₆alkyl or C₃₋₆cycloalkyl;
or R¹⁸ and R²⁰ together with the nitrogen to which they are attached form a heterocyclic 4- to 7-membered ring;
R²¹ and R²² are independently hydrogen, C₁₋₄alkyl, haloC₁₋₄alkyl, aryl and arylC₁₋₄alkyl.

3. A compound according to claim 1 wherein t is 1.

4. A compound according to claim 1 wherein B is phenyl, naphthyl, pyridyl, imidazolyl, quinolinyl, cinnolyl, isoquinolinyl, thienopyridyl, naphthyridinyl, 2,5-methylenedioxyphenyl, 3,4-methylenedioxyphenyl, thienopyrimidinyl, pyrimidinyl, thienyl, pyrrolyl, pyrazolyl, thiazolyl, oxazolyl, isoxazolyl, pyrazinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinolinyl, tetrahydroisoquinolinyl and isoindolinyl, where each is optionally substituted by one or more groups independently selected from nitro, trifluoromethyl, trifluoromethoxy, halo, cyano, C₁₋₄alkyl [symbol] optionally substituted by one or more fluor [symbol] , C₂₋₄alkynyl, heteroaryl, —OR⁹, —NR⁹R¹⁰, —CONR⁹R¹⁰ and —NR⁹COR¹⁰; or B is vinyl or ethynyl optionally substituted by C₁₋₄alkyl; and R⁹ and R¹⁰ are as defined in claim 1.

5. A compound according to claim 1 wherein B is bicyclic aryl, bicyclic heteroaryl or bicyclic heterocyclyl optionally substituted by one or more groups independently selected from nitro, trifluoromethyl, trifluoromethoxy, halo, cyano, C₁₋₄alkyl [symbol] optionally substituted by R⁹ or C₁₋₄alkoxy, or one or more halo [symbol] , C₂₋₄alkenyl [symbol] optionally substituted by halo or R⁹ [symbol] , C₂₋₄alkynyl [symbol] optionally substituted by halo or R⁹ [symbol] , C₃₋₆cycloalkyl [symbol] optionally substituted by R⁹ or one or more halo [symbol] , C₅₋₆cycloalkenyl [symbol] optionally substituted by halo or R⁹ [symbol] , aryl [symbol] optionally substituted by halo or C₁₋₄alkyl [symbol] , heteroaryl [symbol] optionally substituted by halo or C₁₋₄alkyl [symbol] , heterocyclyl [symbol] optionally substituted by C₁₋₄alkyl [symbol] , —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; and R⁹, R¹⁰ and R¹¹ are as defined in claim 1.

6. A compound according to claim 1 wherein B is 2-methylquinolin-4-yl or 2,5-dimethylphenyl.

7. A compound according to claim 2 wherein t is 1 and B is phenyl, naphthyl, pyridyl, imidazolyl, quinolinyl, cinnolyl, isoquinolinyl, thienopyridyl, naphthyridinyl, 2,5-methylenedioxyphenyl, 3,4-methylenedioxyphenyl, thienopyrimidinyl, pyrimidinyl, thienyl, pyrrolyl, pyrazolyl, thiazolyl, oxazolyl, isoxazolyl, pyrazinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinolinyl, tetrahydroisoquinolinyl and isoindolinyl, where each is optionally substituted by one or more groups independently selected from nitro, trifluoromethyl, trifluoromethoxy, halo, cyano, C₁₋₄alkyl [symbol] optionally substituted by one or more fluoro [symbol] , C₂₋₄alkynyl, heteroaryl, —OR⁹, —NR⁹R¹⁰, —CONR⁹R¹⁰ and —NR⁹COR¹⁰; or B is vinyl or ethynyl optionally substituted by C₁₋₄alkyl; and R⁹ and R¹⁰ are as defined in claim 2.

8. A compound according to claim 2 wherein B is a group selected from bicyclic aryl, bicyclic heteroaryl and bicyclic heterocyclyl, where each group is optionally substituted by one or more groups independently selected from nitro, trifluoromethyl, trifluoromethoxy, halo, cyano, C₁₋₄alkyl [symbol] optionally substituted by R⁹ or one or more halo [symbol] , C₂₋₄alkenyl [symbol] optionally substituted by halo or R⁹ [symbol] , C₂₋₄alkynyl [symbol] optionally substituted by halo or R⁹ [symbol] , C₃₋₆cycloalkyl [symbol] optionally substituted by R⁹ or one or more halo [symbol] , C₅₋₆cycloalkenyl [symbol] optionally substituted by halo or R⁹ [symbol] , aryl [symbol] optionally substituted by halo or C₁₋₄alkyl [symbol] , heteroaryl [symbol] optionally substituted by halo or C₁₋₄alkyl [symbol] , heterocyclyl [symbol] optionally substituted by C₁₋₄alkyl [symbol] , —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl, heterocyclyl which group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl or C₁₋₄alkox; and R⁹, R¹⁰ and R¹¹ are as defined in claim 2.

9. A compound according to claim 2 wherein B is 2-methylquinolin-4-yl.

10. A compound according to claim 1 wherein R⁷ is hydrogen or a group selected from C₁₋₄alkyl, arylC₁₋₄alkyl, heteroarylC₁₋₄alkyl, heterocyclylC₁₋₄alkyl, aryl, heteroaryl, heterocyclyl and C₃₋₅cycloalkyl which group is optionally substituted by cyano, C₁₋₄alkyl, halo, —OR²¹, —CO₂R²¹ and —NR²¹CO₂R²².

11. A compound according to claim 1 wherein R⁷ is hydrogen or a group selected from C₁₋₄alkyl, tetrahydrofuranyl, tetrahydropyranyl, pyrrolidinyl, piperidinyl, morpholinyl optionally substituted by one or more C₁₋₄alkoxy, fluoro, —COC₁₋₃alkyl or —SO₂C₁₋₃alkyl.

12. A compound according to claim 1 wherein R⁷ is C₁₋₄alkyl optionally substituted by halo, hydroxy, C₁₋₄alkoxy or amino.

13-14. (canceled)

15. A method of treating inflammatory diseases, autoimmune disease, allergic/atopic diseases, transplant rejection, graft versus host disease, cardiovascular disease, reperfusion injury and malignancy which comprises administering a compound according to claim 1.

16. A pharmaceutical composition comprising a compound according to claim 1 and a pharmaceutically-acceptable diluent or carrier.

17. A process for preparing a compound according to claim 1 comprising the steps of converting a ketone or aldehyde of formula (IIA) or (IIB) into a compound of formula (IA) or (IB); 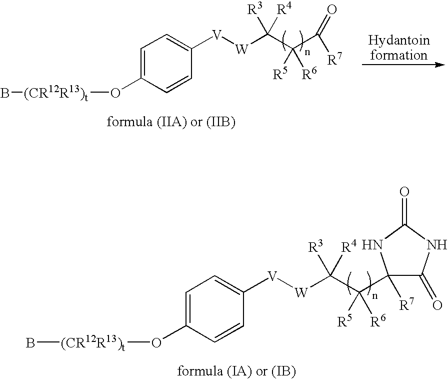
and thereafter if necessary:
i) converting a compound of the formula (IA) or (IB) into another compound of the formula (IA) or (IB);
ii) removing any protecting groups;
iii) forming a pharmaceutically acceptable salt or in vivo hydrolysable ester.

18. A process for preparing a compound according to claim 1 which when W is NR¹ comprises: 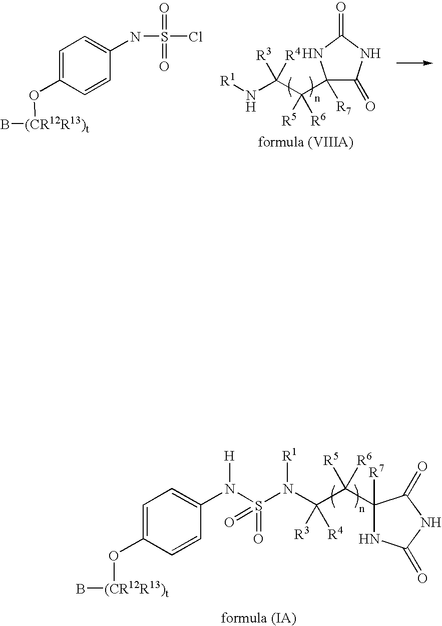
reaction of an amine of formula (VIIIA) with a suitable chlorosulphonamide intermediate under standard sulphonamide formation conditions; or
when W is a bond or CR¹R², comprises 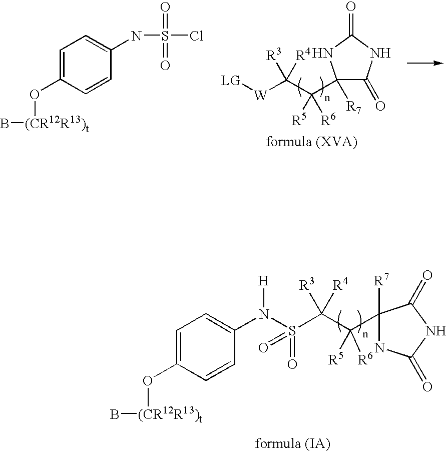
reaction of a hydantoin sulphonyl chloride of formula (XVA) with a suitable chlorosulphonamide intermediate under standard sulphonamide formation conditions;
and thereafter if necessary:
i) converting a compound of the formula (IA) into another compound of the formula (IA);
ii) removing any protecting groups;
iii) forming a pharmaceutically acceptable salt or in vivo hydrolysable ester.
